(19) United States
(12) Patent Application Publication
(10) Pub. No.: US 2024/0139241 A1
(43) Pub. Date: May 2, 2024

(54) COMPOSITIONS AND METHODS FOR CONDITIONING PATIENTS FOR CELL THERAPY

(71) Applicant: Myeloid Therapeutics, Inc., Cambridge, MA (US)

(72) Inventors: Daniel GETTS, Stow, MA (US); Bruce J. McCreedy, JR., Raleigh, NC (US); Michele Luise Gerber, Cambridge, MA (US); Thomas Etienne Prod’homme, Medford, MA (US)

(21) Appl. No.: 18/492,345

(22) Filed: Oct. 23, 2023

Related U.S. Application Data
(63) Continuation of application No. PCT/US2022/026016, filed on Apr. 22, 2022.
(60) Provisional application No. 63/178,897, filed on Apr. 23, 2021.
(60) Provisional application No. 63/220,188, filed on Jul. 9, 2021.
(60) Provisional application No. 63/277,476, filed on Nov. 9, 2021.

(51) Int. Cl. A61K 35/15; A61K 31/4995; A61K 31/675; A61K 31/7076; A61K 39/00; A61P 35/00; C07K 16/28

(52) U.S. Cl. CPC A61K 35/15; A61K 31/4995; A61K 31/675; A61K 31/7076; A61K 39/4614; A61K 39/464429; A61P 35/00; C07K 16/2896; A61K 2239/15; A61K 2239/21; A61K 2239/22; A61K 2239/48

(57) ABSTRACT

Compositions and methods for therapeutic use of engineered myeloid cells are described. Methods for increasing therapeutic effectiveness of immune cells by use of an immune cell inhibitory agent prior to therapy is described. Effective use of myeloid cells in combination therapy is described.

### CROSS REFERENCE

This Application is a continuation of International Patent Application No. PCT/US2022/026016 filed on Apr. 22, 2022, which claims the benefit of U.S. Provisional Application No. 63/178,897, filed on Apr. 23, 2021, U.S. Provisional Application No. 63/220,188, filed on Jul. 9, 2021, and U.S. Provisional Application No. 63/277,476, filed on Nov. 9, 2021, each of which is incorporated herein by reference in its entirety.

### SEQUENCE LISTING STATEMENT

This Application contains a Sequence Listing which has been submitted electronically in XML file format is hereby incorporated by reference in its entirety. Said XML copy, created on Oct. 23, 2023, is named 56371-721.301_SL.xml and is 72,564 bytes in size.

### BACKGROUND

Cellular immunotherapy is a promising new tool for fighting difficult to treat diseases, such as cancer, and persistent infections and also certain diseases that are refractory to other forms of treatment. However, current approaches to cellular immunotherapy that often show great promise in preclinical trials rarely show efficacy in clinical trials, and it is possible that unforeseen influences within a patient's system, affects its effectivity to a great extent. Therefore, to make cellular therapy work effectively, a patient that is the recipient of the immunotherapy may have to be first pre-treated or pre-conditioned to receive the therapy.

Accordingly, there is a need for preparing a patient's system as a whole and/or the disease microenvironment not only to be receptive of a cellular therapy, but also to allow an effective and persistent functioning of the therapeutic cells in the microenvironment. The present invention addresses this need and provides related advantages as well.

### SUMMARY

Provided herein is a pharmaceutical composition formulated for use in treating a disease in a human subject in need thereof that has been treated with an immune cell inhibitory agent, the pharmaceutical composition comprising: (I) a population of cells comprising a therapeutically effective amount of monocytes comprising a recombinant polynucleic acid, wherein the recombinant polynucleic acid comprises a sequence encoding a chimeric fusion protein (CFP), the CFP comprising: (i) an extracellular domain comprising an antigen binding domain, and (ii) a transmembrane domain operatively linked to the extracellular domain; and (II) a pharmaceutically acceptable carrier. In some embodiments, the immune cell inhibitory agent reduces the number of immune cells of the subject or inhibits a function of immune cells of the subject.

Provided herein is a method of treating a disease in a human subject in need thereof that has been treated with an immune cell inhibitory agent, the method comprising administering to the human subject a pharmaceutical composition comprising: (I) a population of cells comprising a therapeutically effective amount of monocytes comprising a recombinant polynucleic acid, wherein the recombinant polynucleic acid comprises a sequence encoding a chimeric fusion protein (CFP), the CFP comprising: (i) an extracellular domain comprising an antigen binding domain, and (ii) a transmembrane domain operatively linked to the extracellular domain; and (II) a pharmaceutically acceptable carrier.

In some embodiments, the method comprises administering the immune cell inhibitory agent to the human subject.

In some embodiments, the immune cell inhibitory agent reduces the number of immune cells of the subject or inhibits a function of immune cells of the subject.

In some embodiments, a dose of the population of cells comprising a therapeutically effective amount of monocytes administered to the human subject is less than a dose of the population of cells comprising a therapeutically effective amount of monocytes administered to a human subject that has not been treated with the immune cell inhibitory agent.

In some embodiments, the immune cell inhibitory agent has been administered or is administered before administering the pharmaceutical composition. In some embodiments, the immune cell inhibitory agent has been administered or is administered about 1, 2, 3, 4, 5, 6, 7, 8, 9, 11, 11, 12, 13, 14, 15, 16, 17 18, 19, 20, 21, 22, 23 or 24 hours before administering the pharmaceutical composition, or about 1, 2, 3, 4, 5, 6, 7, 8, 9, 11, 11, 12, 13 or 14 days before administering the pharmaceutical composition. In some embodiments, the pharmaceutical composition is administered to the human subject within about 1, 2, 3, 4, 5, 6, 7, 8, 9, 11, 11, 12, 13, 14, 15, 16, 17 18, 19, 20, 21, 22, 23 or 24 hours from the time the human subject was administered the immune cell inhibitory agent, or within about 1, 2, 3, 4, 5, 6, 7, 8, 9, 11, 11, 12, 13 or 14 days from the time the human subject was administered the immune cell inhibitory agent. In some embodiments, the immune cell inhibitory agent has been administered or is administered on the same day or at the same time as the pharmaceutical composition.

In some embodiments, the monocytes comprise CD14+ cells, M1 macrophage, M2 macrophages or mosaic myeloid cells/macrophages.

In some embodiments, the immune cell inhibitory agent is an antibody, a small molecule, a lipid, a carbohydrate, a nanoparticle or a peptide.

In some embodiments, the immune cell inhibitory agent inhibits a function of the immune cell, wherein the function is cell migration and/or recruitment to a site or a tissue.

In some embodiments, the immune cell inhibitory agent inhibits and/or binds to a chemokine or a chemokine receptor. In some embodiments, the immune cell inhibitory agent inhibits and/or binds to CCL2, CCL3, CCL7, CCL19, CCL21, CCL24, CCL25, CXCL8, CXCL11, CXCL12, XCL2, CCL3L1, CCR2 or CXCR4. In some embodiments, the immune cell inhibitory agent inhibits binding of CXCL12 to CXCR4. In some embodiments, the immune cell inhibitory agent inhibits binding of CCL2 to CCR2.

In some embodiments, the immune cell inhibitory agent inhibits and/or binds to a cytokine or cytokine receptor. In some embodiments, the immune cell inhibitory agent inhibits and/or binds to CSF1R or CSF1.

In some embodiments of the pharmaceutical composition or of the method described herein, the immune cell inhibitory agent is pexidartinib (PLX3397), ARRY-382, PLX7486, BLZ945, JNJ-41346527, emactuzumab, AMG821, IMC-CS4, cabiralizumab, MCS110 or PD-1361324.

In some embodiments, the immune cell inhibitory agent is a myeloid cell checkpoint inhibitor. In some embodiments, the immune cell inhibitory agent a checkpoint inhibitor that is directed to overcome immune resistance in cancer. In some embodiments, the immune cell inhibitory agent is a stromal checkpoint inhibitor. In some embodiments, the immune cell inhibitory agent is an inhibitor of leukocyte-Associated Immunoglobulin-Like Receptor 1 (LAIR1). In some embodiments, the immune cell inhibitory agent is an agent that inhibits LAIR1. In some embodiments, the immune cell inhibitory agent is a monoclonal antibody that binds to LAIR1. In some embodiments, the LAIR1 inhibitor is used in combination with one or more other immune cell inhibitory agents.

In some embodiments, the immune cell inhibitory agent inhibits and/or binds to a myeloid cell-specific receptor.

In some embodiments, the myeloid cell-specific receptor is CD33, TREM-1 or TREM-2. In some embodiments, wherein the immune cell inhibitory agent inhibits and/or binds to CD33. In some embodiments, the immune cell inhibitory agent is vadastuximab or gemtuzumab.

In some embodiments, the immune cell inhibitory agent inhibits and/or binds to TREM-1 or TREM-2. In some embodiments, the immune cell inhibitory agent is a peptide, an siRNA, or an antibody or a fragment thereof. In some embodiments, the immune cell inhibitory agent is an anti-TREM-2 antibody. In some embodiments, the anti-TREM-2 antibody is PY314.

In some embodiments, the immune cell inhibitory agent induces apoptosis of immune cells. In some embodiments, the immune cell inhibitory agent activates a caspase. In some embodiments, the immune cell inhibitory agent activates caspase 8.

In some embodiments, the immune cell inhibitory agent is a bisphosphonate. In some embodiments, the immune cell inhibitory agent is carlumab, clodronate, ibandronate, pamidronate or zoledronic acid.

In some embodiments, the immune cell inhibitory agent inhibits anaplastic lymphoma kinase (ALK), or binds to ALK tyrosine kinase receptor.

In some embodiments, the immune cell inhibitory agent is trabectedin or lurbinectedin.

In some embodiments, the immune cell inhibitory agent is an HDAC inhibitor.

In some embodiments, the immune cell inhibitory agent is romidepsin.

In some embodiments, the immune cell inhibitory agent is a PARP inhibitor.

In some embodiments, the immune cell inhibitory agent is a VEGF inhibitor.

In some embodiments, the VEGF inhibitor is a kinase inhibitor, including but not limited to—regorafenib, caozantinib, lenvatinib, varditinib. In some embodiments, the immune cell inhibitory agent is a VEGFR inhibitor. In some embodiments, the immune cell inhibitory agent is an antibody. In some embodiments, the immune cell inhibitory agent is tivozanib, or ramucirumab. In some embodiments, the immune cell inhibitory agent is ziv-aflibercept.

In some embodiments, the immune cell inhibitory agent is a CD47 inhibitor.

In some embodiments, the immune cell inhibitory agent is a SIRPα, antagonist.

In some embodiments, the immune cell inhibitory agent inhibits recruitment of immune cells.

In some embodiments, the immune cells are monocytes, macrophages or T cells.

In some embodiments of the pharmaceutical composition or of the method described herein, the monocytes are circulating monocytes.

In some embodiments, the macrophages are tumor associated macrophages (TAMs) or macrophages in a tumor microenvironment.

In some embodiments, the T cells are circulating T cells.

In some embodiments, the immune cell inhibitory agent inhibits recruitment of immune cells to a tumor or a tumor microenvironment.

In some embodiments, the immune cell inhibitory agent reduces the number of immune cells of the subject.

In some embodiments, the immune cells are monocytes, macrophages or T cells.

In some embodiments, the monocytes are circulating monocytes.

In some embodiments, the macrophages are tumor associated macrophages (TAMs) or macrophages in a tumor microenvironment.

In some embodiments, the T cells are circulating T cells.

In some embodiments, the immune cell inhibitory agent promotes immune cell apoptosis.

In some embodiments, the immune cell inhibitory agent is a VEGF inhibitor.

In some embodiments, the immune cell inhibitory agent is a VEGF-A inhibitor.

In some embodiments, the immune cell inhibitory agent is monoclonal antibody to VEGF.

In some embodiments, the monoclonal antibody is bevacizumab.

In some embodiments, the immune cell inhibitory agent is a VEGF receptor (VEGFR) inhibitor.

In some embodiments, the immune cell inhibitory agent is a VEGFR1/2/3 inhibitor.

In some embodiments, the immune cell inhibitory agent is a TAM reprograming agent. In some embodiments, the immune cell inhibitory agent is an immune cell inhibitory agent that is a LAIR1 inhibitor. In some embodiments, the immune cell inhibitory agent is a monoclonal antibody to LAIR1.

In some embodiments, the immune cell inhibitory agent is a small molecule.

In some embodiments, the immune cell inhibitory agent is a small molecule selected from sunitinib, axitinib, and sorafenib.

In some embodiments, the extracellular antigen binding domain comprises a CD5 binding domain.

In some embodiments, the transmembrane domain comprises a CD8 transmembrane domain or a CD68 transmembrane domain or a CD28 transmembrane domain.

In some embodiments, the CFP further comprises an intracellular domain comprising an intracellular signaling domain.

In some embodiments, intracellular domain comprises a PI3K recruitment domain. In some embodiments, intracellular domain comprises a FcR intracellular signaling domain. In some embodiments, the FcR intracellular signaling domain comprises an FcRg intracellular signaling domain or an FcRe intracellular signaling domain.

In some embodiments, the intracellular domain comprises a CD3 zeta domain.

In some embodiments, the population of cells comprising a therapeutically effective amount of monocytes comprises at least about 2×10⁶ monocytes.

In some embodiments, the population of cells comprising a therapeutically effective amount of monocytes comprises CD14+/CD16− cells, CD14+CD16+ cells, CD14^{dim}mCD16+ cells and/or CD14−CD16+ cells.

In some embodiments, the population of cells expressing the CFP comprises at least 50% CD14+/CD16− cells, e.g. at the time of administration.

In some embodiments, the recombinant polynucleic acid is an electroporated recombinant polynucleic acid. In some embodiments, the recombinant polynucleic acid is mRNA.

In some embodiments, the disease is cancer. In some embodiments, the cancer is a CD5+ cancer. In some embodiments, the cancer is a lymphoma. In some embodiments, the lymphoma is a T cell lymphoma In some embodiments, the T cell lymphoma is peripheral T cell lymphoma. In some embodiments, the peripheral T cell lymphoma is a CD5⁺ relapsed/refractory peripheral T cell lymphoma.

In some embodiments, the disease is a cancer, wherein the cancer is a HER2+ cancer. In some embodiments, the cancer is a solid tumor.

In some embodiments, the subject is a human.

In some embodiments, the subject is 18 years of age or older.

In some embodiments, the subject is administered 2, 3, 4, 5, 6, or 7 doses of the pharmaceutical composition over a 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 day period or longer.

In some embodiments, the pharmaceutical composition comprises a dose of about 1.5×11⁸ monocytes. In some embodiments, the immune cell inhibitory agent is administered at least 1 day prior to administering the pharmaceutical composition.

In some embodiments, the immune cell inhibitory agent is administered 2, 3, 4, 5, 6, 7, 8, 9, 10, 11, 12, 13, 14, 15, 16, 17, 18, 19, 20, or 21 days prior to administering the pharmaceutical composition.

In some embodiments, the immune cell inhibitory agent is administered once prior to administering the pharmaceutical composition.

In some embodiments, the immune cell inhibitory agent is administered more than once prior to administering the pharmaceutical composition.

In some embodiments, the pharmaceutical composition comprises a dose of at least about, at most about or about 5×10⁷ monocytes or myeloid cells. In some embodiments, the conditioning agent is administered at least 1 day post administration of the pharmaceutical composition.

In some embodiments, the immune cell inhibitory agent is administered 2, 3, 4, 5, 6, 7, 8, 9, 10, 11, 12, 13, 14, 15, 16, 17, 18, 19, 20, or 21 days after administration of the pharmaceutical composition.

In some embodiments, the immune cell inhibitory agent is administered once after administration of the pharmaceutical composition.

In some embodiments, the immune cell inhibitory agent is administered more than once post administering the pharmaceutical composition.

In some embodiments, the conditioning agent is administered more than once prior and post administering the pharmaceutical composition.

In some embodiments, the immune cell inhibitory agent comprises romidepsin, wherein the romidepsin is administered intravenously over a 4-hour period on days 1, 8 and 15 of a 28-day cycle.

In some embodiments, the immune cell inhibitory agent comprises fludarabine, wherein the fludarabine is administered for 2, 3, 4, 5, 6, or 7 days prior to administering the pharmaceutical composition.

In some embodiments, the immune cell inhibitory agent comprises fludarabine and cyclophosphamide, and wherein the fludarabine is administered for 2, 3, 4, 5, 6, or 7 days prior to administering the pharmaceutical composition and the cyclophosphamide is administered for 1, 2, 3, 4, or 5 days prior to administering the pharmaceutical composition.

In some embodiments, the immune cell inhibitory agent comprises cyclophosphamide, wherein the cyclophosphamide is administered for 1, 2, 3, 4, or 5 days prior to administering the pharmaceutical composition.

In some embodiments, the subject is administered 25 mg/m² fludarabine and 500 mg/m² cyclophosphamide on Days −5 through −3 prior to administering the pharmaceutical composition.

In some embodiments, the immune cell inhibitory agent comprises prednisolone, wherein the prednisolone is administered for 2, 3, 4, 5, 6, 7, 8, 9, 11, 11, 12, 13, 14, 15, 16, 17 18, 19, or 21 days before administering the pharmaceutical composition, or on the same day or at the same time the pharmaceutical composition is administered.

In some embodiments, two or more immune cell inhibitory agents are administered to the subject.

In some embodiments, the immune cell inhibitory agent is a conditioning agent that comprises an FDA approved VEGF inhibitor, wherein the VEGF inhibitor is administered as prescribed by a legally authorized trained practitioner.

In some embodiments, the conditioning agent comprises a VEGF inhibitor, wherein the VEGF inhibitor administration is initiated 2, 3, 4, 5, 6, 7, 8, 9, 11, 11, 12, 13, 14, 15, 16, 17 18, 19, or 21 weeks before administering the pharmaceutical composition, or on the same day or at the same time the pharmaceutical composition is administered.

In some embodiments, the conditioning agent comprises a VEGF inhibitor, wherein the VEGF inhibitor administration is initiated 2, 3, 4, 5, 6, 7, 8, 9, 11, 11, 12, 13, 14, 15, 16, 17 18, 19, or more weeks post administering the pharmaceutical composition, or on the same day or at the same time the pharmaceutical composition is administered.

In some embodiments, the immune cell inhibitor inhibits an immune cell. In some embodiments, the immune cell inhibitor does not inhibit an immune cell. In some embodiments, the immune cell inhibitor modulates an immune cell, such as an activity of an immune cell or expression of a protein of an immune cell. In some embodiments, the immune cell inhibitor is an immune cell modulator. In some embodiments, the immune cell inhibitor is selected from the group consisting of a TLR-agonist, a phagocytosis inhibitor, a DICER inhibitor, an HDAC inhibitor, a PI3-Kinase inhibitor and a myeloid cell surface binding agent.

In some embodiments, the myeloid cell surface binding agent is a lectin binding agent, or a MARCO binding agent. In some embodiments, the myeloid cell surface binding agent is a TREM-1 binding agent.

In some embodiments, the myeloid cell surface binding agent is a checkpoint inhibitor. In some embodiments, the myeloid cell surface binding agent is a an agent that binds to PDL-1, LILRB/ILT or a P selectin. In some embodiments, the myeloid cell surface binding agent is an inhibitor of PDL-1, LILRB/ILT or a P selectin.

In some embodiments, the immune cell inhibitor is a cytokine. In some embodiments, the immune cell inhibitor is a IL-10, TGF-b, IL-4, an anti-CD41 agent, an anti-PD1 agent or an arginase inhibitor.

In some embodiments, the human subject has been lymphodepleted prior to administration of the population of cells.

In some embodiments, the population of cells is autologous or from the human subject.

In some embodiments, the population of cells is allogeneic. In some embodiments, the population of cells is from a healthy donor. In some embodiments, the population of cells is a population of non-engineered cells. In some embodiments, the population of cells is a population of cells with an HLA haplotype matched to the HLA haplotype of the human subject. In some embodiments, the population of cells is a population of cells with an HLA haplotype that is not matched to the HLA haplotype of the human subject.

In some embodiments, the population of cells is derived from a population of genetically modified cells. In some embodiments, the population of genetically modified cells has been genetically engineered to lack expression of one or more HLA alleles, one or more class I HLA alleles, or all class I HLA alleles. In some embodiments, the population of cells is derived from a population of genetically modified stem cells. In some embodiments, the population of genetically modified stem cells is a population of genetically modified pluripotent stem cells. In some embodiments, the population of genetically modified pluripotent stem cells is a population of genetically modified induced pluripotent stem cells (iPSCs).

In some embodiments, the method further comprises administering a second dose of the population of cells. In some embodiments, the population of cells of the second dose is autologous or from the human subject. In some embodiments, a first dose of the population of cells is allogeneic. In some embodiments, the population of cells of the second dose is allogeneic. In some embodiments, a first dose of the population of cells is allogeneic. In some embodiments, the population of cells of the second dose that is allogeneic is HLA-type mismatched to HLA-type of the population of cells of the first dose that is allogeneic. In some embodiments, the human subject elicits an immune response to the population of cells of the first dose that is allogeneic. In some embodiments, the method further comprises administering 1, 2, 3, 4, 5, 6, 7, 8, 9 or 10 additional doses of the population of cells.

Provided herein is a method of treating a peripheral T cell lymphoma (PTCL) in a subject, comprising administering to the subject a composition comprising therapeutically effective number of myeloid cells comprising engineered myeloid cells, the engineered myeloid cells comprising a recombinant nucleic acid encoding a chimeric fusion protein (CFP), wherein the CFP comprises: (i) an extracellular CD5 antigen binding domain that can bind to a CD5 antigen on a cell; wherein the extracellular CD5 antigen binding domain comprises a humanized CD5-specific single-chain fragment variable (scFv) comprising one or more variable domains or humanized variable domains of a H65 murine monoclonal antibody; (ii) a CD8 hinge domain, (iii) a CD8 transmembrane domain and (iv) an intracellular domain comprising a fragment crystallizable gamma (Fcγ) intracellular domain or fragment thereof, and a phosphoinositide 3-kinase (PI3K) signaling domain or fragment thereof from a CD19 intracellular signaling domain.

In some embodiments, the myeloid cells are autologous myeloid cells.

In some embodiments, the autologous myeloid cells are engineered ex vivo.

In some embodiments, the myeloid cells are allogenic myeloid cells.

In some embodiments, the engineered myeloid cells are allogenic myeloid cells engineered ex vivo.

In some embodiments, the therapeutically effective number of myeloid cells is about 0.5×10\hat{ }6 myeloid cells to about 10\hat{ }9 myeloid cells.

In some embodiments, the therapeutically effective number of myeloid cells is about 0.5×10\hat{ }6 to about 0.5×10\hat{ }8 cells.

In some embodiments, the therapeutically effective number of myeloid cells is administered to the subject as an infusion.

In some embodiments, the therapeutically effective number of myeloid cells is administered to the subject as a single dose of an infusion.

In some embodiments, the therapeutically effective number of myeloid cells is administered to the subject as a multiple doses of infusions.

In some embodiments, the therapeutically effective number of myeloid cells is about 4.8×10\hat{ }8 cells per infusion.

In some embodiments, the PTCL is PTCL-NOS, an AITL, or an ALCL (ALK+ or ALK−).

In some embodiments, the PTCL is follicular T cell lymphoma.

In some embodiments, the PTCL is a nodal T cell lymphoma.

In some embodiments, the PTCL is CD5+ relapsed/refractory PTCL.

In some embodiments, the subject is administered 6 doses over three weeks.

In some embodiments, the administering is continued past three weeks.

In some embodiments, an objective response rate (ORR) is noted for each treated subject at 6 months after the first dose of the treatment, wherein the objective response rate is the number (%) of subjects achieving best overall response of complete response or partial response by Lugano Classification criteria, as measured by PET/CT or CT scans and bone marrow biopsy.

In some embodiments, any duration of response (DOR) is noted for each treated subject over 48 weeks after the first dose of the treatment, wherein the DOR is the time interval between the date of first assessment of PR or CR to the date of the follow-on first documentation of progressive disease or death for a subject exhibiting a complete response by Lugano Classification criteria. Other measures of response may include are: progression free survival and overall survival.

In one aspect, provided herein is a method of treating a tumor in a human subject in need thereof, the method comprising: (a) administering to the subject, a first therapeutic regimen comprising engineered myeloid cells over a first period of time; (b) administering to the subject a second therapeutic for a second period of time.

In some embodiments, the second therapeutic is a CAR-T therapy, a checkpoint inhibitor therapy, a monoclonal antibody therapy of a multi-specific antibody therapy.

In some embodiments, the administering (a) and (b) potentiates an immune response parameter that is substantially greater than administering either (a) alone or (b) alone.

In some embodiments, the immune response parameter is cytokine secretion or target cell cytotoxicity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1A-1D depict schematic diagram of four modalities of myeloid cell therapeutics discussed herein. FIG. 1A depicts an exemplary recombinant construct that can be administered in a subject, which when expressed by a myeloid cell in vivo can induce enhanced target-specific phagocytosis and lysis of target cells. FIG. 1B depicts a chimeric fusion protein, representing a chimeric antigen receptor for phagocytosis (CAR-P) encoded by a recombinant polynucleotide, e.g. as shown in FIG. 1A. FIG. 1C depicts exemplary chimeric fusion proteins representing a tri-specific myeloid cell engager (TriME, left) or a bi-specific myeloid cell engager (BiME, right), designed to be encoded by a recombinant polynucleotide. FIG. 1D depicts an engineered myeloid cell expressing a recombinant chimeric antigen receptor for phagocytosis (CAR-P) construct. The detailed diagram on the right exemplifies a mode of action in binding to a target cell (cancer cell) and inducing phagocytosis. The mode of action of an engager molecule is also depicted graphically in the figure.

FIG. 2 depicts two exemplary strategies for reducing tumor associated monocytes/macrophages.

FIG. 3 depicts an exemplary strategy for reducing Tregs.

FIG. 4. Key elements of a clinical study design, phase 1.

FIG. 5. Key elements of a clinical study design, phase 2.

FIG. 6 shows representative data indicating enhanced phagocytosis of tumor cells by CD14+ myeloid cells expressing a CD5-CFP, when combined with anti-CD47 antibody.

FIG. 7 shows prophetic results of a combination therapy using myeloid cell therapy with a VEGF inhibitor on tumor size in a mouse xenograph model.

FIG. 8 shows a prophetic results of a combination therapy using myeloid cell therapy with CAR-T cell therapy on tumor size in a mouse xenograph model.

### DETAILED DESCRIPTION

The diverse functionality of myeloid cells makes them an ideal cell therapy candidate that can be engineered to have numerous therapeutic effects. The present disclosure is related to immunotherapy using myeloid cells (e.g., CD14+ cells) of the immune system, particularly antigen presenting cells (APCs). A number of therapeutic indications could be contemplated using myeloid cells. For example, myeloid cell immunotherapy could be exceedingly important in cancer, autoimmunity, fibrotic diseases and infections. The present disclosure is related to immunotherapy using myeloid cells, including APCs e.g., macrophages, that are modified ex vivo. It is an object of the invention disclosed herein to harness one or more of these functions of myeloid cells for therapeutic uses. For example, it is an object of the invention disclosed herein to harness the antigen presenting activity of myeloid cells, including engineered myeloid cells, for therapeutic uses. For example, it is an object of the invention disclosed herein to harness the ability of myeloid cells, including engineered myeloid cells that have been modified ex vivo, to induce antigen specific tolerance of T cells. For example, it is an object of the invention disclosed herein to harness the ability of myeloid cells, including engineered myeloid cells, to promote activation of tolerogenic APCs, e.g., dendritic cells (DCs). For example, it is an object of the invention disclosed herein to harness the ability of myeloid cells, including engineered myeloid cells, to reduce recruitment and trafficking of immune cells and molecules.

In some embodiments, the present disclosure involves making and using engineered myeloid cells (e.g., CD14+ cells, such as macrophages or other APCs, which can be introduced into a tissue to induce antigen-specific tolerance. Engineered myeloid cells, such as macrophages and other phagocytic cells, can be prepared by incorporating nucleic acid sequences (e.g., mRNA, plasmids, viral constructs) encoding a chimeric fusion protein (CFP), that has an extracellular binding domain specific to disease associated antigens (e.g., autoimmune antigens), into the cells using, for example, recombinant nucleic acid technology, synthetic nucleic acids, gene editing techniques (e.g., CRISPR), transduction (e.g., using viral constructs), electroporation, or nucleofection. It has been found that myeloid cells can be engineered to have a broad and diverse range of activities. For example, it has been found that myeloid cells can be engineered to express a recombinant polynucleotide encoding one or more antigens to have a broad and diverse range of activities. For example, it has been found that myeloid cells can be engineered to harbor a recombinant nucleic acid that encodes one or more antigens, e.g., autoimmune antigens, such that upon introduction into the body of a subject, the myeloid cells induce tolerogenic response against the one or more antigens in the subject, where the subject had exhibited an increased immunogenic response to at least one of the one or more antigen prior to the introducing the engineered myeloid cells. In some embodiments, the myeloid cells can be engineered to promote secretion of tolerogenic molecules such that upon introducing the engineered cells in a subject exhibiting a pathologically increased immune activation prior to the administering of the engineered myeloid cells, and whereupon introducing the engineered cells in the subject, reduces or ameliorates the pathologically increased immune activation. A pathologically increased immune response as used herein can be described as an undesired immune response against a self-antigen, e.g., an autoantigen; or against a non-self-antigen, such as in a grafted tissue in a graft versus host immune response, or such as in a host versus graft immune response; or can be described as an undesired and/or uncontrolled immune response, such as for example, an allergic response, a hyperactive immune response e.g., cytokine storm, or an immune sequelae against a foreign antigen. A person of skill in the art can envisage situations encompassed broadly as a pathologically increased immune response, even if not articulated herein. In some embodiments, the engineered myeloid cells promote secretion of tolerogenic molecules in an inflamed or allergic tissue of a subject. The engineered myeloid cells can be engineered to suppress or reduce recruitment and trafficking of immune cells and molecules responsive to an antigen to a tissue exhibiting an aberrant immune activation or aberrant immune response. An aberrant immune response as described herein can be described as an undesired immune response against a self-antigen, e.g., an autoantigen; or against a non-self-antigen, such as in a grafted tissue in a graft versus host immune response, or such as in a host versus graft immune response; or can be described as an undesired and/or uncontrolled immune response, such as for example, an allergic response, a hyperactive immune response e.g., cytokine storm, or an immune sequelae against a foreign antigen. A person of skill in the art can envisage situations encompassed broadly as a pathologically increased immune response, even if not articulated herein.

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. The present disclosure provides innovative methods and compositions that can successfully transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity. In some embodiments, myeloid cells are manipulated ex vivo, such that upon introducing into a subject. the manipulated myeloid cells comprising the genetic modification are readily taken up by active phagocytes in vivo, which then process antigens comprised in the manipulated engineered myeloid cells, e.g., the one or more antigens encoded by the recombinant polynucleotide in the engineered myeloid cells, as described in the previous paragraphs, and display on the membrane surface, and induce tolerance against the antigens in vivo.

All terms are intended to be understood as they would be understood by a person skilled in the art. Unless defined otherwise, all technical and scientific terms used herein have the same meaning as commonly understood by one of ordinary skill in the art to which the disclosure pertains. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment. As used herein, the singular forms “a,” “an,” and “the” include the plural referents unless the context clearly indicates otherwise. The terms “include,” “such as,” and the like are intended to convey inclusion without limitation, unless otherwise specifically indicated.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

The term “about” or “approximately” as used herein when referring to a measurable value such as a parameter, an amount, a temporal duration, and the like, is meant to encompass variations of +/−30% or less, +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

An “alteration” or “change” can refer to an increase or decrease. For example, an alteration can be an increase or decrease of 1%, 2%, 3%, 4%, 5%, 10%, 20%, 30%, or by 40%, 50%, 60%, or even by as much as 70%, 75%, 80%, 90%, or 100%. For example, an alteration can be an increase or decrease of 1-fold, 2-fold, 3-fold, 4-fold, 5-fold, 10-fold, 20-fold, 30-fold, or by 40-fold, 50-fold, 60-fold, or even by as much as 70-fold, 75-fold, 80-fold, 90-fold, or 100-fold.

An “antigen presenting cell” or “APC” as used herein includes, but is not limited to, professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

As used herein, the term “anergy” can refer to the state of unresponsiveness to antigen stimulation resulting from incomplete or insufficient signals delivered through the T-cell receptor. T cell anergy can also result upon stimulation with antigen in the absence of co-stimulation, resulting in the cell becoming refractory to subsequent activation by the antigen even in the context of costimulation. The unresponsive state can often be overridden by the presence of interleukin-2. Anergic T-cells do not undergo clonal expansion and/or acquire effector functions. For example, anergy in T cells can be characterized by lack of cytokine production, e.g., IL-2. T-cell anergy can occur when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) can result in failure to produce cytokines and subsequently failure to proliferate. Anergic T cells may, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used, for example IL-2 gene transcription induced by a heterologous promoter under the control of a 5′ IL-2 gene enhancer or by a multimer of the AP 1 sequence that can be found within the enhancer (Kang et al. 1992 Science. 257: 1134).

The term “antibody” includes, without limitation, an immunoglobulin which binds specifically to an antigen, including, but not limited to IgG1, IgG2, IgG3, and IgG4), IgA (including IgA1 and IgA2), IgD, IgE, IgM, and IgY. Antibodies include, but are not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a V_{L} and/or a V_{H} domain. Antibodies can be from any animal origin. Antigen-binding antibody fragments, including single-chain antibodies, can comprise variable region(s) alone or in combination with tone or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

An “antigen (Ag)” can refer to any molecule that provokes an immune response or is capable of being bound by an antibody. The immune response may involve either antibody production, or the activation of specific immunologically-competent cells, or both. A person of skill in the art would readily understand that any macromolecule, including virtually all proteins or peptides, can serve as an antigen. An antigen can be endogenously expressed or can be recombinantly expressed. An antigen can be specific to a certain tissue, such as a cancer cell, or it can be broadly expressed. In addition, fragments of larger molecules can act as antigens. In one embodiment, antigens are tumor antigens.

Engineered myeloid cells can be included in a therapeutic cell product for use. ATAK myeloid cells, as used herein, can be engineered myeloid cells. Engineered myeloid cells can comprise a recombinant polynucleic acid encoding a chimeric antigen receptor comprising an antigen binding extracellular domain targeted towards an antigen on a diseased cell, for example, a cancer cell. The target antigen may be a cancer antigen. In one aspect, the present disclosure involves making and using engineered myeloid cells (e.g., CD14+ cells, such as macrophages or other phagocytic cells, which can attack and kill (ATAK) diseased cells directly and/or indirectly, such as cancer cells and infected cells. Engineered myeloid cells, such as macrophages and other phagocytic cells, can be prepared by incorporating nucleic acid sequences (e.g., mRNA, DNA, plasmids, viral constructs) encoding a chimeric fusion protein (CFP), that has an extracellular binding domain specific to disease associated antigens (e.g., cancer antigens), into the cells using, for example, recombinant nucleic acid technology, synthetic nucleic acids, gene editing techniques (e.g., CRISPR), transduction (e.g., using viral constructs), electroporation, or nucleofection. It has been found that myeloid cells can be engineered to have a broad and diverse range of activities. For example, it has been found that myeloid cells can be engineered to express a chimeric fusion protein (CFP) containing an antigen binding domain to have a broad and diverse range of activities. For example, it has been found that myeloid cells can be engineered to have enhanced phagocytic activity such that upon binding of the CFP to an antigen on a target cell, the cell exhibits increased phagocytosis of the target cell. It has also been found that myeloid cells can be engineered to promote T cell activation such that upon binding of the CFP to an antigen on a target cell, the cell promotes activation of T cells, such as T cells in the tumor microenvironment. The engineered myeloid cells can be engineered to promote secretion of tumoricidal molecules such that upon binding of the CFP to an antigen on a target cell, the cell promotes secretion of tumoricidal molecules from nearby cells. The engineered myeloid cells can be engineered to promote recruitment and trafficking of immune cells and molecules such that upon binding of the CFP to an antigen on a target cell, the cell promotes recruitment and trafficking of immune cells and molecules to the target cell or a tumor microenvironment. In one aspect, provided herein are various examples of recombinant polynucleic acids encoding a chimeric fusion protein comprising (a) a transmembrane domain and (b) an intracellular domain operably linked to the transmembrane domain, wherein the chimeric fusion protein responds to an extracellular cue, wherein the intracellular domain influences the intracellular mechanism of action and activation of the myeloid cell upon receiving the extracellular cue. In one embodiment, the chimeric fusion protein is a chimeric receptor, having an extracellular antigen binding domain in addition to (a) a transmembrane domain and (b) an intracellular domain operably linked to the transmembrane domain, and engagement of the extracellular antigen binding domain of the chimeric fusion protein to the target antigen that the extracellular binding domain binds to provides the extracellular cue to the receptor and for the receptor mediated activation of the myeloid cell. ATAK myeloid cells are not necessarily limited to therapeutic use against a cancer cell, but can be variously engineered to suit the need for diseases other than cancer. Engineered myeloid cells can be used to treat a number of therapeutic indications. For example, engineered myeloid cells immunotherapy can be used to treat cancer, autoimmunity, fibrotic diseases and/or infections. The present disclosure is related to immunotherapy using myeloid cells, such as phagocytic cells of the immune system, for example, monocytes. An object of the invention disclosed herein can be to harness one or more of these functions of myeloid cells for therapeutic uses. For example, an object of the invention disclosed herein can be to harness the phagocytic activity of myeloid cells, including engineered myeloid cells, for therapeutic uses. For example, an object of the invention disclosed herein can be to harness the ability of myeloid cells, including engineered myeloid cells, to promote T cell activation. For example, an object of the invention disclosed herein can be to harness the ability of myeloid cells, including engineered myeloid cells, to promote secretion of tumoricidal molecules. For example, an object of the invention disclosed herein can be to harness the ability of myeloid cells, including engineered myeloid cells, to promote recruitment and trafficking of immune cells and molecules. In one aspect, the disclosure provides new and useful chimeric constructs for expression in a myeloid cell. When expressed the chimeric construct is expressed by a myeloid cell, the myeloid can target a target molecule or a cell that comprises the target molecule, e.g., a target antigen on a surface pf a cell. One of the many facets of the present disclosure is to (i) enhance a therapeutic activity, such as phagocytic activity of the myeloid cells (e.g., the engineered myeloid cells expressing a chimeric construct); and/or to initiate a coordinated and sustained immune response against the target (e.g., target antigen). Innovative methods and compositions can be successfully employed to transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity. The resultant cells can be therapeutically effective engineered myeloid cells, or effector myeloid cells. In some embodiments, an engineered cell is a CD14+ cell. In some embodiments, an engineered cell is a CD14+/CD16− cell. In some embodiments, an engineered cell is a CD14+/MHCII+ cell. In some embodiments, an engineered cell is a CD14+/CD16− cell, isolated from the blood by negative selection e.g., using antibodies against cell-surface molecules that bind to cells that are thereafter removed leaving the desired cell untouched, which are then collected. In some embodiments, an engineered cell, such as an engineered monocyte is prepared from cells isolated from leukapheresis samples. In some embodiments, an engineered cell, such as an engineered monocyte, is autologous to the patient.

A “biological sample” can refer to any tissue, cell, fluid, or other material derived from an organism.

The “control elements” or “regulatory sequences” can be present in an expression vector and can refer to non-translated regions of the vector, including, but not limited to, origins of replication, selection cassettes, promoters, enhancers, translation initiation signals (Shine Dalgarno sequence or Kozak sequence) introns, a polyadenylation sequence, 5′ and 3′ untranslated regions which can interact with host cellular proteins to carry out transcription and translation. Such elements may vary in their strength and specificity. Depending on the vector system and host utilized, any number of suitable transcription and translation elements, including ubiquitous promoters and inducible promoters may be used. In particular embodiments, a vector for use in practicing the invention including, but not limited to expression vectors and viral vectors, includes exogenous, endogenous, or heterologous control sequences such as promoters and/or enhancers.

Percent identity in the context of two or more nucleic acids or polypeptide sequences can refer to two or more sequences that are the same. Two sequences can be “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity can exist over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates can be designated, if necessary, and sequence algorithm program parameters can be designated. Default program parameters can be used, or alternative parameters can be designated. The sequence comparison algorithm can then calculate the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. The algorithm parameters for using nucleotide BLAST to determine nucleotide sequence identity may use scoring parameters with a match/mismatch score of 1,-2 and wherein the gap costs are linear. The length of the sequence that initiates an alignment or the word size in a BLAST algorithm may be set to 28 for sequence alignment. The algorithm parameters for using protein BLAST to determine a peptide sequence identity may use scoring parameters with a BLOSUM62 matrix to assign a score for aligning pairs of residues, and determining overall alignment score, wherein the gap costs may have an existence penalty of 11 and an extension penalty of 1. The matrix adjustment method to compensate for amino acid composition of sequences may be a conditional compositional score matrix adjustment. The length of the sequence that initiates an alignment or the word size in a BLAST algorithm may be set to 6 for sequence alignment.

Described herein are compositions and methods for the design, preparation, manufacture and/or formulation of circular polynucleotides including circular RNA. As used herein, “circular RNA” or “circRNA” can refer to a single stranded circular polynucleotide which acts substantially like, and has the properties of, an RNA, and that can encode at least one polypeptide of interest. The term “circular” is also meant to encompass any secondary or tertiary configuration of the circular RNA.

The term “chemotaxis” can refer to the directed movement of cells in response to a chemical stimulus (e.g., chemokines). Multiple types of cells have been documented to migrate (e.g., via chemotaxis) into sites (e.g., of inflammation and/or injury) including, but not limited to, monocytes, myeloid cells, macrophages, neutrophils, T cells and natural killer cells.

The term “elutriation” can refer to a purification, separation, or removal process that separates cells based on differences in their density. An exemplary elutriation process to purify human monocytes is centrifugal elutriation.

The term “epitope” can refer to a peptide which is a portion of an antigen, wherein the peptide comprises an amino acid sequence that is capable of stimulating an immune response. Epitopes include any protein determinant capable of specific binding to, for example, an antibody, antibody peptide, an antibody-like molecule, an MHC molecule, or a T cell receptor. For example, MHC class I epitopes may be used to stimulate an immune response. In preferred embodiments, Epitopes include, but are not limited to, peptides non-covalently bound to an MHC molecule on the surface of antigen presenting cells in a manner which facilitates its interaction with T-cell receptors (TCR).

The term “electroporation” can refer to the temporary creation of holes or aqueous pores in the surface of a cell membrane by an applied electrical potential, for example, through which materials or agents, such as nucleic acids, may pass into the cell. Exemplary conditions used for electroporation include selection of voltage used, pulse width and number of pulses. Typically, a voltage in the range of about 800 V/cm-1400 V/cm is applied in a pulse of about 8 milliseconds-15 milliseconds. More than one pulse can be applied, typically 1-3 pulses are applied. Particular conditions selected can depend on variables such as cell type, size and species from which the cell is derived and such conditions can be selected by one of skill in the art. Electroporation methods are well-known in the art, for example, as described in J. A. Nickoloff, Animal Cell Electroporation and Electrofusion Protocols, Humana Press; 1st ed., 1995.

Those of ordinary skill in the art, reading the present disclosure, will appreciate that the term “engineered”, as used herein, can refer to an aspect of having been manipulated and altered by the hand of man. In particular, the term “engineered cell” can refer to a cell that has been subjected to a manipulation, such that its genetic, epigenetic, and/or phenotypic identity is altered relative to an appropriate reference cell such as otherwise identical cell that has not been manipulated as such. In some embodiments, the manipulation is or comprises a genetic manipulation. In some embodiments, a genetic manipulation is or comprises one or more of (i) introduction of a nucleic acid not present in the cell prior to the manipulation (i.e., of a heterologous nucleic acid); (ii) removal of a nucleic acid, or portion thereof, present in the cell prior to the manipulation; and/or (iii) alteration (e.g., by sequence substitution) of a nucleic acid, or portion thereof, present in the cell prior to the manipulation. In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. Those of ordinary skill in the art will appreciate that reference to an “engineered cell” herein may, in some embodiments, encompass both the particular cell to which the manipulation was applied and also any progeny of such cell. An engineered cell, such as an engineered monocyte, can refer to a cell that has at least one exogenous nucleic acid sequence in the cell, even if transiently expressed. Expressing an exogenous nucleic acid may be performed by various methods described elsewhere, and encompasses methods known in the art. Aspects of the present disclosure relate to preparing and using engineered cells, for example, engineered myeloid cells, such as engineered phagocytic cells. Aspects of the present disclosure relate to, inter alia, an engineered cell comprising an exogenous nucleic acid encoding, for example, a chimeric fusion protein (CFP).

The term “fusion polypeptide” can refer to a polypeptide comprising at least two polypeptides, e.g. proteins, protein domains, or parts thereof, linked covalently, preferably by a peptide bond. A chimeric fusion protein includes, but is not limited to, a fusion polypeptide (e.g., a fusion protein), where one or more components from two or more heterogenous proteins or polypeptides are fused to form the fusion polypeptide. In some embodiments, a chimeric fusion protein is a engineered to be expressed on the surface of the cell. In some embodiments, a chimeric fusion protein may be a recombinant receptor protein. In some embodiments, the chimeric fusion protein may comprise an antigen binding domain that binds to an antigen, wherein the antigen is expressed by a target cell or presented by an antigen presenting cell. A chimeric fusion polypeptide (CFP) can be encoded by a recombinant polynucleic acid (may interchangeably be termed nucleic acid, or recombinant polynucleotide), such as DNA or RNA.

The term “enhancer” can refer to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” can refer to a segment of DNA which contains sequences capable of providing both promoter and enhancer functions.

A “fragment” can refer to a portion of a protein or nucleic acid. In some embodiments, a fragment retains at least 50%, 75%, 80%, 90%, 95%, 99% or 100% of the biological activity of a reference protein or nucleic acid.

Term “Kozak sequence” can refer to a short nucleotide sequence that facilitates the initial binding of mRNA to the small subunit of the ribosome and increases translation. An exemplary consensus Kozak sequence is (GCC)RCCATGG, where R is a purine (A or G) (Kozak, 1986. Cell. 44(2):283-92, and Kozak, 1987. Nucleic Acids Res. 15(20):8125-48).

As used herein, an “internal ribosome entry site” or “IRES” can refer to an element that promotes direct internal ribosome entry to the initiation codon, such as ATG, of a cistron (a protein encoding region), thereby leading to the cap-independent translation of the gene. See, e.g., Jackson et al, 1990. Trends Biochem Sci 15(12):477-83) and Jackson and Kaminski. 1995. RNA 1(10):985-1000. An IRES can allow for 5′-end/cap-independent initiation of translation and can provide the ability to express 2 proteins from a single messenger RNA (mRNA) molecule. IRESs are commonly located in the 5′ UTR of positive-stranded RNA viruses with uncapped genomes. Another exemplary means to express 2 proteins from a single mRNA molecule is by insertion of a 2A peptide(-like) sequence in between their coding sequence. 2A peptide(-like) sequences mediate self-processing of primary translation products by a process variously referred to as “ribosome skipping”, “stop-go” translation and “stop carry-on” translation. 2A peptide(-like) sequences are present in various groups of positive- and double-stranded RNA viruses including Picornaviridae, Flaviviridae, Tetraviridae, Dicistroviridae, Reoviridae and Totiviridae.

M1 macrophages, M1 monocytes, or M1 cell types of the monocyte lineage can refer to lineage subtypes that exhibit a pro-inflammatory and/or anti-tumor phenotype. M2 monocytes, M2 macrophages or M2 cell types of the monocyte lineage can refer to lineage subtypes that exhibit an anti-inflammatory and/o pro-tumor phenotype.

As used herein, the term “operably linked” can refer to a functional or structural relationship between two or more segments, such as nucleic acid segments or polypeptide segments.

As used herein, the term “immune response” can refer to a response elicited in an animal. In some embodiments, an immune response may refer to cellular immunity, humoral immunity or may involve both. In some embodiments, an immune response may be limited to a part of the immune system. For example, in certain embodiments, an immune response comprise an increased IFNy response. In certain embodiments, immune response may comprise mucosal IgA response (e.g., as measured in nasal and/or rectal washes). In certain embodiments, an immune response may be or comprise a systemic IgG response (e.g., as measured in serum). In certain embodiments, an immune response may be or comprise a neutralizing antibody response. In certain embodiments, an immune response may be or comprise a cytolytic (CTL) response by T cells. In certain embodiments, an immune response may be or comprise reduction in immune cell activity. The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages Immune responses include adaptive immune responses. The adaptive immune system can react to foreign molecular structures, such as antigens of an intruding organism. Unlike the innate immune system, the adaptive immune system can be highly specific to a pathogen. Adaptive immunity can also provide long-lasting protection. Adaptive immune reactions include humoral immune reactions and cell-mediated immune reactions. In humoral immune reactions, antibodies secreted by B cells into bodily fluids can bind to pathogen-derived antigens leading to elimination of the pathogen through a variety of mechanisms, e.g. complement-mediated lysis. In cell-mediated immune reactions, T cells capable of destroying other cells may be activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

An “immune cell inhibitory agent” includes, but is not limited to, an agent that modulates an immune cell or an activity of an immune cell or an immune response or a response to an agent, such as a therapeutic agent. In some embodiments, an immune cell inhibitory agent is an agent that inhibits or dampens an immune cell or an immune response or a response to an agent. In some embodiments, an immune cell inhibitory agent is an agent that activates or enhances an immune cell or an activity of an immune cell or an immune response or a response to an agent. In some embodiments, an immune cell inhibitory agent is a biological or chemical agent, e.g., a small molecule, an antibody, an antibody fragment, a synthetic molecule, a monoclonal antibody, a drug, an inhibitor, or an activator, which when administered to a human subject, can affect an immune cell in vivo, and/or can alter an immune environment of the subject, at least temporarily. In some embodiments, an immune cell inhibitory agent is synthetic In some embodiments, an immune cell inhibitory agent may be used to generate a window conducive for an effective action of a therapeutic, such as a drug, a monoclonal antibody or another immune cell. For example, the immune cell inhibitory agent can specifically block cell division, proliferation, extravasation or chemotaxis of an immune cell type, such as myeloid cell, a lymphocyte, etc. For example, an immune cell inhibitory agent can be a lymphodepleting agent. For example, an immune cell inhibitory agent can be a myelo-depleting agent. An exemplary immune cell inhibitory agent is fludarabine. Another exemplary immune cell inhibitory agent is cyclophosphamide. In some instances, an immune cell inhibitory agent can be an immunomodulator, or an immunosuppressor. In some instances, an immune cell inhibitory agent can be a cytokine. In some instances, an immune cell inhibitory agent can be tissue specific. In some instances, a function of an immune cell inhibitory agent is temporary and/or reversible. In some embodiments, the effect of the immune cell inhibitory agent, such as an anti-CD47 agent, can potentiate and/or activate an immune cell function, for example, myeloid cell mediated phagocytosis. In some embodiments, an immune cell inhibitory agent is a conditioning agent or a preconditioning agent. In some embodiments, an immune cell inhibitory agent can be used to condition an immune system for enhancing the effect of a therapy. In some embodiments, an immune cell inhibitory agent is a tumor microenvironment (TAM) reprograming agent.

The term “major histocompatibility complex (MHC)”, “MHC molecule”, or “MHC protein” can refer to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins can be loaded with antigens that usually originate from endogenous proteins or from pathogens present inside cells, and are then presented to naïve or cytotoxic T-lymphocytes (CTLs). HLA class II proteins can be present on antigen presenting cells (APCs), including but not limited to dendritic cells, B cells, and macrophages. They can be present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes which can fuse with lysosomes whose acidic enzymes cleave the uptaken protein into many different peptides. Autophagy can be another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α-and β-chains that interact to form a peptide-binding groove that is more open than class I peptide-binding grooves.

HLA alleles can typically be expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRα (DRA1), and one or more genes HLA-DRβ (DRB1 and DRB3, −4 or −5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

Nucleic acid molecules useful in the methods of the disclosure include, but are not limited to, any nucleic acid molecule with activity or a sequence that encodes a polypeptide. Polynucleotides having substantial identity to an endogenous sequence are typically capable of hybridizing with at least one strand of a double-stranded nucleic acid molecule. “Hybridize” can refer to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency can be accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions can be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Phagocytosis” is used interchangeably with “engulfment” and can refer to a process by which a cell engulfs a particle or cell, such as a cancer cell or an infected cell. This process can give rise to an internal compartment (phagosome) containing the particle or cell. This process can be used to ingest and or remove a particle, such as a cancer cell or an infected cell from the body.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a recombinant nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties. In some cases, a polypeptide may be used interchangeably with the term “protein”.

The expression “pharmaceutically acceptable” can refer to the ingredients of a pharmaceutical composition are compatible with each other and not deleterious to the subject to which it is administered. The expression “pharmaceutically acceptable carrier, diluent, excipient and/or adjuvant” can refer to a substance that does not substantially produce an adverse, allergic or other untoward reaction when administered to an animal, preferably a human. The term includes, but is not limited to, inactive substances such as for example solvents, cosolvents, antioxidants, surfactants, stabilizing agents, emulsifying agents, buffering agents, pH modifying agents, preserving agents (or preservating agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g., FDA Office or EMA.

The term “polyadenylation signal”, poly(A) signal” or “poly(A) site” as used herein denotes a genetic element which directs both the termination and polyadenylation of a nascent RNA transcript. The term “poly(A) sequence” as used herein can denote a DNA sequence associated with the termination and polyadenylation of a nascent RNA transcript. Efficient polyadenylation of the recombinant transcript is desirable, as transcripts lacking a poly(A) tail can be unstable and are rapidly degraded. The poly(A) signal utilized in an expression vector may be “heterologous” or “endogenous.” An endogenous poly(A) signal can be one that is found naturally at the 3′ end of the coding region of a given gene in the genome. A heterologous poly(A) signal can be one which has been isolated from one gene and positioned 3′ to another gene.

The term “recombinant nucleic acid” can refer to a nucleic acid prepared, expressed, created or isolated by recombinant means. A recombinant nucleic acid can contain a nucleotide sequence that is not naturally occurring. The term “recombinant nucleic acid” may be interchangeably used with “recombinant polynucleotide” throughout the document, and is understood in this context to mean the same. A recombinant nucleic acid may be synthesized in the laboratory. A recombinant nucleic acid may be prepared by using recombinant DNA technology, for example, enzymatic modification of DNA, such as enzymatic restriction digestion, ligation, and DNA cloning. A recombinant nucleic acid can be DNA, RNA, analogues thereof, or a combination thereof. A recombinant DNA may be transcribed ex vivo or in vitro, such as to generate a messenger RNA (mRNA). A recombinant mRNA may be isolated, purified and used to transfect a cell. A recombinant nucleic acid may encode a protein or a polypeptide. The process of introducing or incorporating a nucleic acid into a cell can be via transformation, transfection or transduction. Transformation is the process of uptake of foreign nucleic acid by a bacterial cell. This process can be adapted for propagation of plasmid DNA, protein production, and other applications. Transformation introduces recombinant plasmid DNA into competent bacterial cells that take up extracellular DNA from the environment. Some bacterial species can be naturally competent under certain environmental conditions, but competence is artificially induced in a laboratory setting. Transfection can be the method of introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ refers to natural infections of humans or animals with wild-type viruses.

“Substantially identical” can refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity can be typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” can be a standard of comparison. It will be understood that the numbering of the specific positions or residues in the respective sequences can be dependent on the particular protein and/or numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

The terms “spacer” or “linker” as used in reference to a fusion protein can refer to a peptide sequence that joins two other peptide sequences of the fusion protein. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

“Recruitment” as used herein, can refer to cell recruitment to a site of infection, inflammation or a tumor. Recruitment of immune cells to a site of infection, inflammation or a tumor, for example can be a result of chemotaxis where a chemoattractant released at or from the site activates receptors resulting in extravasation of the immune cells and migration to the site. Migration of immune cells to the site can lead to an immune response to a tumor or an agent, e.g., a pathogen, exacerbation of or resolution of inflammation, an immune response and/or resolution of an infection or inhibition of tumor growth.

The term “subject” or “patient” can refer to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline. In some embodiments, a subject or a patient is a human.

The term “substantially higher than” or “substantially greater than” is used as the same meaning as considerably higher than or considerably greater than. For example, when comparing the cytokine release from a cell before and after a treatment, the cytokine level may be found considerably higher or substantially higher after treatment compared to the value before treatment, if the value after the treatment is higher/greater than the value before treatment by (say) at least 10%, 20%, 50%, several folds . . . etc., and/or is statistically significant as determined by known methods in the art. In other words, the change is not considered to be trivial or dismissive.

The term “therapeutic effect” can refer to some extent of relief of one or more of the symptoms of a disorder (e.g., a neoplasia, tumor, or infection by an infectious agent or an autoimmune disease) or its associated pathology. “Therapeutically effective amount” as used herein can refer to an amount of an agent which is effective, upon single or multiple dose administration to the cell or subject, in prolonging the survivability of the patient with such a disorder, reducing one or more signs or symptoms of the disorder, preventing or delaying, or the like beyond that expected in the absence of such treatment. “Therapeutically effective amount” may be intended to qualify the amount required to achieve a therapeutic effect. A physician or veterinarian having ordinary skill in the art can readily determine and prescribe the “therapeutically effective amount” (e.g., ED50) of the pharmaceutical composition required.

The term “vector” can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein can be an expression vector. Expression vectors are capable of directing the expression of genes and/or nucleic acid sequence to which they are operatively linked. In some embodiments, an expression vector or plasmid can be in the form of circular double stranded DNA molecules. A vector or plasmid may or may not be integrated into the genome of a host cell. In some embodiments, nucleic acid sequences of a plasmid may not be integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector can be a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

### Preconditioning for Myeloid Cell Therapy

Tumor Associated Macrophages (TAMs)—Macrophages have been described as responding to this micromilieu with either a pro-inflammatory or an anti-inflammatory phenotype (also referred to as “fight” versus “fix” macrophages, respectively). In early stage as well as metastatic cancer, the dominant tumor-associated macrophage (TAM) phenotype is reported to be anti-inflammatory, immune-regulatory, and therefore tumor-promoting (also termed alternatively activated or M2 macrophages) as opposed to pro-inflammatory and tumoricidal (classically activated or M1 macrophages). TAM infiltration has been shown to have a negative prognostic relevance in most tumor types. This phenotype is a consequence of the continuous presence of growth factors such as colony-stimulating factor-1 (CSF1; or macrophage colony-stimulating factor [MCSF]) as well as the cluster of differentiation (CD)-4⁺ type 2 helper T-cell-derived (Tₕ2) cytokines interleukin (IL)-4, IL-13, and IL-10 in the TME. In contrast, M1 macrophages are ascribed tumoricidal functions and are generated in the presence of granulocyte-macrophage colony-stimulating factor (GM-CSF or CSF2) and pro-inflammatory stimuli such as interferon (IFN)-γ, lipopolysaccharide, or tumor necrosis factor α.

Macrophage polarization within the tumor microenvironment (TME) is highly dependent on the local cytokine milieu which originates either from tumor cells, other stromal cells such that immune cells or fibroblasts, as well as macrophage themselves. The M2 TAM phenotype is a consequence of the continuous presence of growth factors such as colony-stimulating factor-1 (CSF-1) as well as CD4+ T cell-derived TH2 cytokines IL-4, IL-10 and IL-13.M2 TAMs directly promote tumor growth. M2-TAM also efficiently suppress immune effector functions that are able to contribute to tumor cell elimination. On the other hand, the silencing effector immune cells is achieved by producing cytokines and enzymes that may directly suppress effector cells or indirectly via other immune cells such as intratumoral DCs, Tregs and type 2 helper T cells. In contrast, M1 TAMs are often attributed to tumoricidal functions via generation of GM-CSF and pro-inflammatory stimuli like IFNg, LPS or TNF-alpha.

The immuno-suppressive and pro-angiogenic micro-environment may be the physiological result of a process of prolonged inflammation and continuous tissue damage and remodeling. Tumor cells and immune cells in the TME produce cytokines, growth factors, and metabolites, which promote the pro-tumor polarization of TAMs. Biological mediators, such as CSF-1, CCL2, and vascular endothelial growth factor (VEGF), promote the accumulation of TAMs in the TME. The Th2 cytokines IL-4, IL-13, IL-10, and TGF produced by Treg and TAMs are key drivers of immune-suppression. Acidification of the TME caused by lactate derived from enhanced glycolytic activity of cancer cells induces regulatory macrophages through G protein-couple receptor (GPCR) and IL-1 beta-converting enzyme (ICE), enhances VEGF and arginase expression, thus promoting M2-like features of TAMs

The hypoxia-inducible factor 1 (HIF-alpha) is a master transcriptional regulator of cellular response to low oxygen concentration Indeed, the ability of different cells to sense and adapt to oxygen availability has been recognized by the Nobel Prize in Physiology and Medicine in 2019, and this also applies to TAMs. Especially in advanced tumors, TAMs accumulate in hypoxic areas; these TAMs are MHC^{low}, have pro-angiogenic behavior and poor antigen-presenting ability; on the other hand, macrophages localized in areas of normoxia, may be more heterogeneous, and some of them may present an M1 orientation with MHC^{high} expression Hypoxic TAMs upregulate REDD1, and endogenous inhibitor of MTORC1, leading to a decrease in glucose intake by TAMs and to higher availability for endothelial cells, thus promoting neo-angiogenesis and metastasis. TAMs overexpress the PD-1 ligands (PD-L1 and PD-L2), as well as the CTLA-4 ligand.

CSF1 receptor (CSF1R)-mediated signaling is crucial for the differentiation and survival of the mononuclear phagocyte system and macrophages in particular. CSF1R belongs to the type III protein tyrosine kinase receptor family, and binding of CSF1 or the more recently identified ligand, IL-34, induces homodimerization of the receptor and subsequent activation of receptor signaling. As the intratumoral presence of CSF1R⁺ macrophages correlates with poor survival in various tumor types, targeting CSF1R signaling in tumor-promoting TAM represents an attractive strategy to eliminate or repolarize these cells. In addition to TAM, CSF1R expression can be detected on other myeloid cells within the tumor microenvironment such as dendritic cells, neutrophils, and myeloid-derived suppressor cells (MDSCs).

Tumors, like persistent infections, create a microenvironment for itself that is immunosuppressive. Tumor associated macrophages undergo M2 polarization and become tumor permissive instead of attacking the tumor cells and being tumor suppressive. Likewise, generation of regulatory T cells (Tregs) help the tumor by inducing immunotolerance to tumor antigens. In order for a cell therapy to be effective, a preconditioning the tumor microenvironment (or, for other suitable disease such as persistent infections), may be necessary. In one aspect, a preconditioning of the host, that is, the human subject that is the recipient of the myeloid cell therapy, comprises administering preconditioning agent, wherein the preconditioning agent is an immune cell modulatory agent, such as an immune cell inhibitory agent. In some embodiments, the therapeutic regime comprises administering a first composition, comprising an immune cell inhibitory agent.

The instant disclosure is directed to generating compositions and methods for effective therapy using myeloid cells that are engineered to express a chimeric fusion protein (CFP), and that enhances myeloid cell mediated cytotoxicity to tumor cells, and enhancing an immune response by activating other immune cells. Primarily, the composition for therapy comprises CD14+ myeloid cells comprising a recombinant polynucleic acid encoding the CFP. In other embodiments, the CD14+ myeloid cells could be engineered to express a fusion protein that is secreted in vivo, and activates myeloid cells and other cells in the vicinity, e.g., wherein the fusion protein encodes a bi-specific or tri-specific engager molecule. In yet other embodiments, myeloid cells may be engineered and/or activated in vivo by cell-specific uptake of recombinant polynucleic acid encoding a fusion protein.

In some embodiments, engineered myeloid cells as described in the above paragraph, e.g. ATAK cells, are designed to migrate to tumors, recognize tumor cells through antigen specific binders and kill the tumor cells directly and through activation of the adaptive immune system. Migration of ATAK cells into the tumor is enhanced by the deletion of both circulating monocytes and TAMS. In order to increase the efficacy of the therapeutic approach using myeloid cells, one or more additional agents are considered for use prior to, during or after the treatment using the engineered myeloid cells. The one or more agents may (e.g. temporarily) remove non-efficient resident monocytes or macrophages or myeloid cells or other immune cells that are immunosuppressive and have been negatively influenced by tumor microenvironment, or in turn are pro-tumor and help the tumor survive. For example, Lurbinectedin kills monocytes and TAMs through effect on Caspase 8. It can be hypothesized that the combination of ATAK cells and lurbinectiden delays tumor progression and improves overall survival, e.g., in mice. The combination of ATAK cells and an immune cell inhibitory agent such as lurbinectiden can result in an overall response rate of 50% and a duration of response of 6 months in patients with refractory TCL. The combination of ATAK cells and lurbinectiden can result in overall response rate of 50% and duration of response of 6 months in patients with refractory HER2 overexpressing tumors. Using this rationale above, the therapeutic approach using myeloid cells can be enhanced by using one or more agents that facilitate, augment or prolong the effect of ATAK myeloid cells against a particular cancer.

Accordingly, provided herein is a pharmaceutical composition formulated for use in treating a disease in a human subject in need thereof that has been treated with an immune cell inhibitory agent, the pharmaceutical composition comprising: (I) a population of cells comprising a therapeutically effective amount of monocytes comprising a recombinant polynucleic acid, wherein the recombinant polynucleic acid comprises a sequence encoding a chimeric fusion protein (CFP), the CFP comprising: (i) an extracellular domain comprising an antigen binding domain, and (ii) a transmembrane domain operatively linked to the extracellular domain; and (II) a pharmaceutically acceptable carrier. Accordingly, provided herein is a pharmaceutical composition, comprising: (A) a first composition comprising an immune cell inhibitory agent; (B) a second composition comprising: (I) a population of cells comprising a therapeutically effective amount of monocytes comprising a recombinant polynucleic acid, wherein the recombinant polynucleic acid comprises a sequence encoding a chimeric fusion protein (CFP), the CFP comprising: (i) an extracellular domain comprising an antigen binding domain, and (ii) a transmembrane domain operatively linked to the extracellular domain; and (II) a pharmaceutically acceptable carrier.

In some embodiments, an immune cell inhibitory agent is a TAM modifier, or reprogrammer or TAM. In some embodiments, the immune cell inhibitory agent is used as a preconditioning agent for a cell therapy, e.g., a myeloid cell therapy for a disease, e.g. cancer. In some embodiments, the immune cell inhibitory agent is a TAM reprogramming agent used to precondition a myeloid cell therapy. In some embodiments the TAM reprograming agent e.g. an immune inhibitory agent is used as a preconditioning agent prior to CAR-T cell therapy. In some embodiments the TAM reprograming agent e.g. an immune inhibitory agent is used as a preconditioning agent prior to CAR-NK cell therapy. In some embodiments the TAM reprograming agent e.g. an immune inhibitory agent is used as a preconditioning agent prior to myeloid cell and CAR-T cell combination therapy. In some embodiments, an immune cell inhibitory agent described herein is used as a preconditioning agent prior to a therapy using bispecific myeloid cell engager (BiME) or tri-specific myeloid cell engager (TRiME), or multispecific myeloid cell engager therapy. In some embodiments, an immune cell inhibitory agent is used as a preconditioning agent prior to a therapy using bispecific NK cell engager (BiKE) or tri-specific NK cell engager (TRiKE), or multispecific NK cell engager therapy. In some embodiments, an immune cell inhibitory agent is used as a preconditioning agent prior to a therapy using bispecific T cell engager (BiTE) or tri-specific T cell engager (TRiTE), or multispecific T cell engager therapy.

In some embodiments, a combination therapy regime may be followed that involves a prior myeloid cell therapy described at least in part in the instant disclosure, followed by a T cell therapy as is well known. In some embodiments, a preconditioning agent or a reprogramming agent as described herein is used prior to the myeloid cell therapy or prior to the T cell therapy, or both.

In some embodiments, the combination therapy involves a prior myeloid cell therapy, followed by a NK cell therapy. In some embodiments, a preconditioning agent or a TAM reprogramming agent as described herein is used prior to the myeloid cell therapy or prior to the NK cell therapy, or both. In some embodiments, the combination therapy may involve a prior NK cell therapy followed by or concomitantly with a myeloid cell therapy. In some embodiments, a preconditioning agent or a TAM reprogramming agent as described herein is used prior to the NK cell therapy or prior to the myeloid cell therapy, or prior to the concomitant therapy.

In some embodiments, the combination therapy involves a prior myeloid cell therapy, followed by a an engager therapy. In some embodiments, a preconditioning agent or a reprogramming agent as described herein is used prior to the myeloid cell therapy or prior to the engager therapy, or both.

Preconditioning with Cycloheximide and Fludarabine

In some embodiments, the methods of conditioning a patient in need of a cell therapy, e.g., myeloid cell therapy, comprises administering to the patient a pre-conditioning agent, e.g., an immune cell inhibitory agent, wherein the immune cell inhibitory agent may comprise a combination of cyclophosphamide and fludarabine. Conditioning a patient with between about 200 mg/m²/day and about 2000 mg/m²/day cyclophosphamide and between about 20 mg/m²/day and 900 mg/m²/day fludarabine may be well tolerated and enhances the effectiveness of a myeloid cell therapy that is subsequently administered to the patient, while reducing the occurrence and/or severity of adverse events associated with higher doses of cyclophosphamide and/or fludarabine.

The present disclosure identifies that administration of cyclophosphamide and fludarabine prior to administration of a myeloid cell therapy reduces the number of endogenous lymphocytes.

The endogenous lymphocytes that are reduced can include, but is not limited to, endogenous regulatory T cells, B cells, natural killer cells, CD4+ T cells, CD8+ T cells, or any combination thereof, which can inhibit the anti-tumor effect of adoptively transferred myeloid cells.

In some embodiments, administration of cyclophosphamide and fludarabine enhances an effector function of myeloid cells administered after the conditioning. In some embodiments, administration of cyclophosphamide and fludarabine enhances antigen presenting cell activation and/or availability.

In one embodiment, the instant disclosure includes a method of conditioning a patient in need of a myeloid cell therapy comprising administering to the patient a dose of cyclophosphamide between about 200 mg/m²/day and about 2000 mg/m²/day and a dose of fludarabine between about 20 mg/m²/day and about 900 mg/m²/day. In another embodiment, the instant disclosure includes a method of conditioning a patient in need of a Myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of conditioning a patient in need of a myeloid cell therapy comprising administering to the patient a dose of cyclophosphamide between about 1110 mg/m²/day and about 2000 mg/m²/day and a dose of fludarabine between about 20 mg/m²/day and about 900 mg/m²/day, e.g., 20 mg/m²/day, 25 mg/m²/day, 30 mg/m²/day, or 60 mg/m²/day. In another embodiment, the instant disclosure includes a method of conditioning a patient in need of a Myeloid cell therapy comprising administering to the patient a dose of cyclophosphamide between about 1110 mg/m²/day and about 2000 mg/m²/day and a dose of fludarabine between about 20 mg/m²/day and about 900 mg/m²/day, e.g., 20 mg/m²/day, 25 mg/m²/day, 30 mg/m²/day, or 60 mg/m²/day, wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of conditioning a patient in need of a Myeloid cell therapy comprising administering to the patient a dose of cyclophosphamide equal to or higher than about 30 mg/kg/day and lower than 60 mg/kg/day and a dose of fludarabine between about 20 mg/m²/day and about 900 mg/m²/day, e.g., 20 mg/m²/day, 25 mg/m²/day, 30 mg/m²/day, or 60 mg/m²/day.

In another embodiment, the instant disclosure includes a method of reducing or depleting endogenous lymphocytes in a patient in need of a myeloid cell therapy comprising administering to the patient a dose of cyclophosphamide between about 200 mg/m²/day and about 2000 mg/m²/day and a dose of fludarabine between about 20 m g/m 2/day and about 900 mg/m²/day.

In another embodiment, the instant disclosure includes a method of reducing or depleting endogenous lymphocytes in a patient in need of a myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of reducing or depleting endogenous lymphocytes in a patient in need of a Myeloid cell therapy comprising administering to the patient a dose of cyclophosphamide between about 1110 mg/m²/day and about 2000 mg/m²/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of reducing or depleting endogenous lymphocytes in a patient in need of a Myeloid cell therapy comprising administering to the patient a dose of cyclophosphamide equal to or higher than 30 mg/kg/day and lower than 60 mg/kg/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine.

In other embodiments, the instant disclosure includes a method of increasing the availability of a homeostatic cytokine in a patient in need of a myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day). In another embodiment, the instant disclosure includes a method of increasing the availability of a homeostatic cytokine in a patient in need of a myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of increasing the availability of a homeostatic cytokine in a patient in need of a Myeloid cell therapy comprising administering to the patient a dose of cyclophosphamide between about 1110 mg/m²/day and about 2000 mg/m²/day and a dose of fludarabine between about 20 mg/m²/day and about 900 mg/m²/day (e.g., 20 m g/m 2/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of increasing the availability of a homeostatic cytokine in a patient in need of a Myeloid cell therapy comprising administering to the patient a dose of cyclophosphamide equal to or higher than about 30 mg/kg/day and lower than 60 mg/kg/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine.

In one particular embodiment, the instant disclosure includes a method of enhancing an effector function of administered T cells in a patient in need of a myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day). In another embodiment, the instant disclosure includes a method of enhancing an effector function of administered T cells in a patient in need of a myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of enhancing an effector function of administered T cells in a patient in need of a Myeloid cell therapy comprising administering to the patient a dose of cyclophosphamide between about 1110 mg/m²/day and about 2000 mg/m²/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of enhancing an effector function of administered T cells in a patient in need of a Myeloid cell therapy comprising administering to the patient a dose of cyclophosphamide equal to or higher than about 30 mg/kg/day and lower than 60 mg/kg/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine.

In some embodiments, the instant disclosure includes a method of enhancing antigen presenting cell activation and/or availability in a patient in need of a myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day).

In another embodiment, the instant disclosure includes a method of enhancing antigen presenting cell activation and/or availability in a patient in need of a Myeloid cell therapy comprising administering to the patient a dose of cyclophosphamide between about 200 mg/m²/day and about 2000 mg/m²/day (e.g., 200 mg/m²/day, 300 mg/m²/day, or 500 mg/m²/day) and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of enhancing antigen presenting cell activation and/or availability in a patient in need of a myeloid cell therapy comprising administering to the patient a dose of cyclophosphamide between about 1110 mg/m²/day and about 2000 mg/m²/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine. In one embodiment, the instant disclosure includes a method of enhancing antigen presenting cell activation and/or availability in a patient in need of a Myeloid cell therapy comprising administering to the patient a dose of cyclophosphamide equal to or higher than about 30 mg/kg/day and lower than 60 mg/kg/day and a dose of fludarabine between about 20 mg/m²/day and about 900 mg/m²/day (e.g., 20 mg/m²/day, 25 mg/m²/day, 30 mg/m²/day, or 60 mg/m²/day), wherein the patient exhibits increased serum levels of IL-7, IL-15, IL-10, IL-5, IP-10, IL-8, MCP-1, PLGF, CRP, sICAM-1, sVCAM-1, or any combination thereof, e.g., IL-15, IP-10, and/or IL-7, or decreased serum levels of perforin and/or MIP-1b after the administration of the cyclophosphamide and fludarabine.

The methods of the present disclosure include the administration of cyclophosphamide and fludarabine prior to a Myeloid cell therapy. The timing of the administration of each component can be adjusted to maximize effect. As described herein, the day that a Myeloid cell therapy is administered is designated as day 0. The cyclophosphamide and fludarabine can be administered at any time prior to administration of the Myeloid cell therapy. In some embodiments, the administration of the cyclophosphamide and fludarabine begins at least seven days, at least six days, at least five days, at least four days, at least three days, at least two days, or at least one day prior to the administration of the Myeloid cell therapy. In other embodiments, the administration of the cyclophosphamide and fludarabine begins at least eight days, at least nine days, at least ten days, at least eleven days, at least twelve days, at least thirteen days, or at least fourteen days prior to the administration of the Myeloid cell therapy. In one embodiment, the administration of the cyclophosphamide and fludarabine begins seven days prior to the administration of the Myeloid cell therapy. In another embodiment, the administration of the cyclophosphamide and fludarabine begins five days prior to the administration of the Myeloid cell therapy.

In one particular embodiment, administration of the cyclophosphamide begins about seven days prior to the administration of the Myeloid cell therapy, and the administration of the fludarabine begins about five days prior to the administration of the Myeloid cell therapy. In another embodiment, administration of the cyclophosphamide begins about five days prior to the administration of the Myeloid cell therapy, and the administration of the fludarabine begins about five days prior to the administration of the Myeloid cell therapy. The timing of the administration of each component can be adjusted to maximize effect. In general, the cyclophosphamide and fludarabine can be administered daily. In some embodiments, the cyclophosphamide and fludarabine are administered daily for about two days, for about three days, for about four days, for about five days, for about six days, or for about seven days. In one particular embodiment, the cyclophosphamide is administered daily for 2 days, and the fludarabine is administered daily for five days. In another embodiment, both the cyclophosphamide and the fludarabine are administered daily for about 3 days.

As described herein, the day the Myeloid cell therapy is administered to the patient is designated as day 0. In some embodiments, the cyclophosphamide is administered to the patient on day 7 and day 6 prior to day 0 (i.e., day −7 and day −6). In other embodiments, the cyclophosphamide is administered to the patient on day −5, day −4, and day −3. In some embodiments, the fludarabine is administered to the patient on day −5, day −4, day −3, day −2, and day −1. In other embodiments, the fludarabine is administered to the patient on day −5, day −4, and day −3.

The cyclophosphamide and fludarabine can be administered on the same or different days. If the cyclophosphamide and fludarabine are administered on the same day, the cyclophosphamide can be administered either before or after the fludarabine. In one embodiment, the cyclophosphamide is administered to the patient on day −7 and day −6, and the fludarabine is administered to the patient on day −5, day −4, day −3, day −2, and day −1. In another embodiment, the cyclophosphamide is administered to the patient on day −5, day −4, and day −3, and the fludarabine is administered to the patient on day −5, day −4, and day −3.

In certain embodiments, cyclophosphamide and fludarabine can be administered concurrently or sequentially. In one embodiment, cyclophosphamide is administered to the patient prior to fludarabine. In another embodiment, cyclophosphamide is administered to the patient after fludarabine.

The cyclophosphamide and fludarabine can be administered by any route, including intravenously (IV). In some embodiments, the cyclophosphamide is administered by IV over about 30 minutes, over about 35 minutes, over about 40 minutes, over about 45 minutes, over about 50 minutes, over about 55 minutes, over about 60 minutes, over about 90 minutes, over about 120 minutes. In some embodiments, the fludarabine is administered by IV over about 10 minutes, over about 15 minutes, over about 20 minutes, over about 25 minutes, over about 30 minutes, over about 35 minutes, over about 40 minutes, over about 45 minutes, over about 50 minutes, over about 55 minutes, over about 60 minutes, over about 90 minutes, over about 120 minutes.

In certain embodiments, a Myeloid cell therapy is administered to the patient following administration of cyclophosphamide and fludarabine. In some embodiments, the Myeloid cell therapy comprises an adoptive cell therapy. In certain embodiments, the adoptive cell therapy is selected from tumor-infiltrating lymphocyte (TIL) immunotherapy, autologous cell therapy, engineered autologous cell therapy (eACT), and allogeneic T cell transplantation. In one particular embodiment, the eACT comprises administration of engineered antigen specific chimeric antigen receptor (CAR) positive (+) T cells. In another embodiment, the eACT comprises administration of engineered antigen specific T cell receptor (TCR) positive (+) T cells. In some embodiments the engineered T cells treat a tumor in the patient.

In one particular embodiment, the instant disclosure includes a method of conditioning a patient in need of a Myeloid cell therapy comprising administering to the patient a dose of cyclophosphamide of about 500 m g/m 2/day and a dose of fludarabine of about 60 mg/m²/day, wherein the cyclophosphamide is administered on days −5, −4, and −3, and wherein the fludarabine is administered on days −5, −4, and −3. In another embodiment, the instant disclosure includes a method of conditioning a patient in need of a Myeloid cell therapy comprising administering to the patient a dose of cyclophosphamide of about 500 mg/m²/day and a dose of fludarabine of about 60 mg/m²/day, wherein the cyclophosphamide is administered on days −7 and −6, and wherein the fludarabine is administered on days −5, −4, −3, −2, and −1. In another embodiment, the instant disclosure includes a method of conditioning a patient in need of a Myeloid cell therapy comprising administering to the patient a dose of cyclophosphamide of about 500 mg/m²/day and a dose of fludarabine of about 30 mg/m²/day, wherein the cyclophosphamide is administered on days −7 and −6, and wherein the fludarabine is administered on days −5, −4, −3, −2, and −1. In another embodiment, the instant disclosure includes a method of conditioning a patient in need of a Myeloid cell therapy comprising administering to the patient a dose of cyclophosphamide of about 300 mg/m²/day and a dose of fludarabine of about 60 mg/m²/day, wherein the cyclophosphamide is administered on days −7 and −6, and wherein the fludarabine is administered on days −5, −4, −3, −2, and −1.

Various other interventions may be included in the methods described herein. For example, it is well known that cyclophosphamide and fludarabine may cause adverse events in patients following administration. It is within the scope of the instant disclosure that compositions may also be administered to the patient to reduce some of these adverse events. In some embodiments, the method further comprises administering a saline solution to the patient. The saline solution can be administered to the patient either prior to or after the administration of the cyclophosphamide and/or fludarabine, or both before and after the administration of the cyclophosphamide and/or fludarabine. In certain embodiments, the saline solution can be administered concurrently with the cyclophosphamide and/or fludarabine. In one particular embodiment, saline solution is administered to the patient prior to the administration of cyclophosphamide and/or fludarabine and following the administration of cyclophosphamide and/or fludarabine on the day of each infusion.

The saline solution may be administered to the patient by any route, including, e.g., intravenously or orally. In some embodiments, the method comprises administering about 0.1 L, about 0.2 L, about 0.3 L, about 0.4 L, about 0.5 L, about 0.6 L, about 0.7 L, about 0.8 L, about 0.9 L, about 1 L, about 1.1 L, about 1.2 L, about 1.3 L, about 1.4 L, about 1.5 L, about 1.6 L, about 1.7 L, about 1.8 L, about 1.9 L, or about 2.0 L of saline solution. The NaCl of the saline solution can be dissolved to a final concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%. In one embodiment, the method comprises administering 1.0 L of 0.9% NaCl saline solution to the patient. In one particular embodiment, the method comprises administering 1.0 L of 0.9% NaCl saline solution to the patient prior to the administration of cyclophosphamide and/or fludarabine and following the administration of cyclophosphamide and/or fludarabine on the day of each infusion.

Further, adjuvants and excipients can also be administered to the patient. For example, mesna (sodium 2-sulfanylthanesulfonate) is an adjuvant that acts as a detoxifying agent to inhibit hemorrhagic cystitis and hematuria, which can occur following treatment with cyclophosphamide Cyclophosphamide, in vivo, can be converted to urotoxic metabolites, such as acrolein. Mesna assists to detoxify these metabolites by reaction of its sulfhydryl group with the vinyl group. It also increases urinary excretion of cysteine. In certain embodiments, the method further comprises administering mesna to the patient. The mesna can be administered prior to the administration of the cyclophosphamide and/or fludarabine, after the administration of the cyclophosphamide and/or fludarabine, or both prior to and after the administration of the of the cyclophosphamide and/or fludarabine. In one embodiment, Mesna is administered intravenously or orally (per mouth). For example, oral mesna can be given with oral cyclophosphamide.

In addition, exogenous cytokines may also be administered to the patient in the method described herein. As discussed above, it is hypothesized that reducing the number of endogenous lymphocytes increases the bioavailability of endogenous molecules, such as cytokines, that can favor the expansion, activation, and trafficking of adoptively transferred T cells. Accordingly, various cytokines may be administered to the patient. In one embodiment, the method further comprises administering one or more doses of IL-2, IL-15, IL-7, IL-10, IL-5, IP-10, IL-8, MCP-1, PLGF, CRP, sICAM-1, sVCAM-1, or any combination thereof. In one particular embodiment, the method comprises administering one or more doses of IL-2. The dose of IL-2 can be at least about 10,000 IU/kg, at least about 50,000 IU/kg, at least about 100,000 IU/kg, at least about 200,000 IU/kg, at least about 400,000 IU/kg, at least about 600,000 IU/kg, at least about 700,000 IU/kg, at least about 800,000 IU/kg, or at least about 1,000,000 IU/kg.

Cyclophosphamide (ENDOXAN®, CYTOXAN®, PROCYTOX®, NEOSAR®, REVIMMUNE®, CYCLOBLASTIN®) is a nitrogen mustard-derivative alkylating agent with potent immunosuppressive activity. Cyclophosphamide acts as an antineoplastic, and it is used to treat various types of cancers including lymphoma, multiple myeloma, leukemia, mycosis fungoides, neuroblastoma, ovarian cancer, eye cancer, and breast cancer, as well as autoimmune disorders.

Once administered to a patient, cyclophosphamide is converted into acrolein and phosphoramide in the liver. Together, these metabolites crosslink DNA in both resting and dividing cells by adding an alkyl group to guanine bases of DNA at the number seven nitrogen atom of the imidazole ring. As a result, DNA replication is inhibited leading to cell death.

In some embodiments, the dose of cyclophosphamide can be adjusted depending on the desired effect, e.g., to modulate the reduction of endogenous lymphocytes and/or control the severity of adverse events. For example, the dose of cyclophosphamide can be higher than about 300 mg/m²/day and lower than about 900 mg/m²/day. In some embodiments, the dose of cyclophosphamide is about 350 mg/m²/day-about 2000 mg/m²/day, at least about 400 mg/m²/day-about 2000 mg/m²/day, about 450 mg/m²/day-about 2000 mg/m²/day, about 500 mg/m²/day-about 2000 mg/m²/day, about 550 mg/m²/day-about 2000 mg/m²/day, or about 600 mg/m²/day-about 2000 mg/m²/day. In another embodiment, the dose of cyclophosphamide is about 350 mg/m²/day-about 1500 mg/m²/day, about 350 mg/m²/day-about 1000 mg/m²/day, about 400 mg/m²/day-about 900 mg/m²/day, about 450 mg/m²/day-about 800 mg/m²/day, about 450 mg/m²/day-about 700 mg/m²/day, about 500 mg/m²/day-about 600 mg/m²/day, or about 300 mg/m²/day-about 500 mg/m²/day. In certain embodiments, the dose of cyclophosphamide is about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day. In one particular embodiment, the dose of cyclophosphamide is about 200 mg/m²/day. In one particular embodiment, the dose of cyclophosphamide is about 300 mg/m²/day. In another embodiment, the dose of cyclophosphamide is about 500 mg/m²/day. In other embodiments, the dose of cyclophosphamide is about 200 mg/m²/day-about 2000 mg/m²/day, about 300 mg/m²/day-about 2000 mg/m²/day, about 400 mg/m²/day-about 2000 mg/m²/day, about 500 mg/m²/day-about 2000 mg/m²/day, about 600 mg/m²/day-about 2000 mg/m²/day, about 700 mg/m²/day-about 2000 mg/m²/day, about 800 mg/m²/day-about 2000 mg/m²/day, about 900 mg/m²/day-about 2000 mg/m²/day, about 1000 mg/m²/day-about 2000 mg/m²/day, about 1100 mg/m²/day-about 2000 mg/m²/day, about 1200 mg/m²/day-about 2000 mg/m²/day, about 1300 mg/m²/day-about 2000 mg/m²/day, about 1400 mg/m²/day-about 2000 mg/m²/day, about 1500 mg/m²/day-about 2000 mg/m²/day, about 1600 mg/m²/day-about 2000 mg/m²/day, about 1700 mg/m²/day-about 2000 mg/m²/day, about 1800 mg/m²/day-about 2000 mg/m²/day, about 1900 mg/m²/day-about 2000 mg/m²/day, about 200 mg/m²/day-about 1900 mg/m²/day, about 400 mg/m²/day-about 1800 mg/m²/day, about 500 mg/m²/day-about 1700 mg/m²/day, about 600 mg/m²/day-about 1600 mg/m²/day, about 700 mg/m²/day-about 1500 mg/m²/day, about 800 mg/m²/day-about 1400 mg/m²/day, about 900 mg/m²/day-about 1300 mg/m²/day, about 1000 mg/m²/day-about 1200 mg/m²/day, about 1100 mg/m²/day-about 1200 mg/m²/day, or about 1110 mg/m²/day-about 1150 mg/m²/day.

Fludarabine phosphate (FLUDARA®) is a synthetic purine nucleoside that differs from physiologic nucleosides in that the sugar moiety is arabinose instead of ribose or deoxyribose. Fludarabine acts as a purine antagonist antimetabolite, and it is used to treat various types of hematological malignancies, including various lymphomas and leukemias.

Once administered to a patient, fludarabine is rapidly dephosphorylated to 2-fluoro-ara-A and then phosphorylated intracellularly by deoxycytidine kinase to the active triphosphate, 2-fluoro-ara-ATP. This metabolite then interferes with DNA replication, likely by inhibiting DNA polymerase alpha, ribonucleotide reductase, and DNA primase, thus inhibiting DNA synthesis. As a result, fludarabine administration leads to increased cell death in dividing cells.

In the present disclosure, the dose of fludarabine can also be adjusted depending on the desired effect. For example, the dose of fludarabine can be higher than 30 mg/m²/day and lower than 900 mg/m²/day. In some embodiments, the dose of fludarabine can be about 35 mg/m²/day-about 900 mg/m²/day, about 40 mg/m²/day-about 900 mg/m²/day, about 45 mg/m²/day-about 900 mg/m²/day, about 50 mg/m²/day-about 900 mg/m²/day, about 55 mg/m²/day-about 900 mg/m²/day, or about 60 mg/m²/day-about 900 mg/m²/day. In other embodiments, the dose of fludarabine is about 35 mg/m²/day-about 900 mg/m²/day, about 35 mg/m²/day-about 800 mg/m²/day, about 35 mg/m²/day-about 700 mg/m²/day, about 35 mg/m²/day-about 600 mg/m²/day, about 35 mg/m²/day-about 500 mg/m²/day, about 35 mg/m²/day-about 400 mg/m²/day, about 35 mg/m²/day-about 300 mg/m²/day, about 35 mg/m²/day-about 200 mg/m²/day, about 35 mg/m²/day-about 100 mg/m²/day, about 40 mg/m²/day-about 90 mg/m²/day, about 45 mg/m²/day-about 80 mg/m²/day, about 45 mg/m²/day-about 70 mg/m²/day, or about 50 mg/m²/day-about 60 mg/m²/day. In certain embodiments, the dose of fludarabine is about 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, about 100 mg/m²/day, about 200 mg/m²/day, or about 300 mg/m²/day. In other embodiments, the dose of fludarabine is about 110 mg/m²/day, 120 mg/m²/day, 130 mg/m²/day, 140 mg/m²/day, 150 mg/m²/day, 160 mg/m²/day, 170 mg/m²/day, 180 mg/m²/day, or 190 mg/m²/day. In some embodiments, the dose of fludarabine is about 210 mg/m²/day, 220 mg/m²/day, 230 mg/m²/day, 240 mg/m²/day, 250 m g/m 2/day, 260 mg/m²/day, 270 mg/m²/day, 280 mg/m²/day, or 290 mg/m²/day. In one particular embodiment, the dose of fludarabine is about 20 mg/m²/day. In one particular embodiment, the dose of fludarabine is about 30 mg/m²/day. In another embodiment, the dose of fludarabine is about 60 mg/m²/day. In another embodiment, the dose of fludarabine is about 25 mg/m²/day.

The doses of cyclophosphamide and fludarabine can be raised or lowered together or independently. For example, the dose of cyclophosphamide can be increased while the dose of fludarabine is decreased, and the dose of cyclophosphamide can be decreased while the dose of fludarabine is increased. Alternatively, the dose of both cyclophosphamide and fludarabine can be increased or decreased together.

In some embodiments, the dose of cyclophosphamide is 100 mg/m²/day (or 110 mg/m²/day, 120 mg/m²/day, 130 mg/m²/day, or 140 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 150 mg/m²/day (or 160 mg/m²/day, 170 mg/m²/day, 180 mg/m²/day, or 190 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is about 200 mg/m²/day (or 210 mg/m²/day, 220 mg/m²/day, 230 mg/m²/day, or 240 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 250 mg/m²/day (or 260 mg/m²/day, 270 mg/m²/day, 280 mg/m²/day, or 290 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 300 mg/m²/day (or 310 mg/m²/day, 320 mg/m²/day, 330 mg/m²/day, or 340 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 350 mg/m²/day (or 360 mg/m²/day, 370 mg/m²/day, 380 mg/m²/day, or 390 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 400 mg/m²/day (or 410 mg/m²/day, 420 mg/m²/day, 430 mg/m²/day, or 440 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 450 mg/m²/day (or 460 mg/m²/day, 470 mg/m²/day, 480 mg/m²/day, or 490 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 500 mg/m²/day (or 510 mg/m²/day, 520 mg/m²/day, 530 mg/m²/day, or 540 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 550 mg/m²/day (or 560 mg/m²/day, 570 mg/m²/day, 580 mg/m²/day, or 590 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 600 mg/m²/day (or 610 mg/m²/day, 620 mg/m²/day, 630 mg/m²/day, or 640 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 650 mg/m²/day (or 660 mg/m²/day, 670 mg/m²/day, 680 mg/m²/day, or 690 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 700 mg/m²/day (or 710 mg/m²/day, 720 mg/m²/day, 730 mg/m²/day, or 740 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 750 mg/m²/day (or 760 mg/m²/day, 770 mg/m²/day, 780 mg/m²/day, or 790 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 800 mg/m²/day (or 810 mg/m²/day, 820 mg/m²/day, 830 mg/m²/day, or 840 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 850 mg/m²/day (or 860 mg/m²/day, 870 mg/m²/day, 880 mg/m²/day, or 890 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 900 mg/m²/day (or 910 mg/m²/day, 920 mg/m²/day, 930 mg/m²/day, or 940 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 950 mg/m²/day (or 960 mg/m²/day, 970 mg/m²/day, 980 mg/m²/day, or 990 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments, the dose of cyclophosphamide is 1000 mg/m²/day (or 1010 mg/m²/day, 1020 mg/m²/day, 1030 mg/m²/day, or 1040 mg/m²/day) and the dose of fludarabine is 5 mg/m²/day, 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In other embodiments, the dose of cyclophosphamide is between 100 mg/m²/day and 650 mg/m²/day, and the dose of fludarabine is between 10 mg/m²/day and 50 mg/m²/day. In other embodiments, the dose of cyclophosphamide is between 150 mg/m²/day and 600 mg/m²/day, and the dose of fludarabine is between 20 mg/m²/day and 50 mg/m²/day. In other embodiments, the dose of cyclophosphamide is between 200 mg/m²/day and 550 mg/m²/day, and the dose of fludarabine is between 20 mg/m²/day and 40 mg/m²/day. In other embodiments, the dose of cyclophosphamide is between 250 mg/m²/day and 550 mg/m²/day, and the dose of fludarabine is between 15 mg/m²/day and 45 mg/m²/day.

In certain embodiments, the dose of cyclophosphamide is 1000 mg/m²/day, and the dose of fludarabine is 60 mg/m²/day, 65 mg/m²/day, 70 mg/m²/day, 75 mg/m²/day, 80 mg/m²/day, 85 mg/m²/day, 90 mg/m²/day, 95 mg/m²/day, 100 mg/m²/day, 105 mg/m²/day, 110 mg/m²/day, 115 mg/m²/day, 120 mg/m²/day, 125 mg/m²/day, 130 mg/m²/day, 135 mg/m²/day, 140 mg/m²/day, 145 mg/m²/day, 150 mg/m²/day, 155 mg/m²/day, 160 mg/m²/day, 165 mg/m²/day, 170 mg/m²/day, 175 mg/m²/day, 180 mg/m²/day, 185 mg/m²/day, 190 mg/m²/day, 195 mg/m²/day, 200 mg/m²/day, 205 mg/m²/day, 210 mg/m²/day, 215 mg/m²/day, 220 mg/m²/day, 225 mg/m²/day, 230 mg/m²/day, 235 mg/m²/day, 240 mg/m²/day, 245 mg/m²/day, or 250 mg/m²/day.

In some embodiments, the dose of cyclophosphamide is 200 mg/m²/day and the dose of fludarabine is 20 mg/m²/day. In some embodiments, the dose of cyclophosphamide is 200 mg/m²/day and the dose of fludarabine is 30 mg/m²/day. In some embodiments, the dose of cyclophosphamide is 300 mg/m²/day and the dose of fludarabine is 30 mg/m²/day. In other embodiments, the dose of cyclophosphamide is 300 mg/m²/day and the dose of fludarabine is 60 mg/m²/day. In other embodiments, the dose of cyclophosphamide is 500 mg/m²/day and the dose of fludarabine is 30 mg/m²/day. In still other embodiments, the dose of cyclophosphamide is 500 mg/m²/day and the dose of fludarabine is 60 mg/m²/day. In some embodiments, the dose of cyclophosphamide is about 1110 mg/m²/day and the dose of fludarabine is 25 mg/m²/day. In some embodiments, the dose of cyclophosphamide is about 2000 mg/m²/day and the dose of fludarabine is 25 mg/m²/day. In some embodiments, the dose of cyclophosphamide is 30 mg/kg/day and the dose of fludarabine is 25 mg/m²/day.

### Preconditioning to Modulate Myeloid Cells

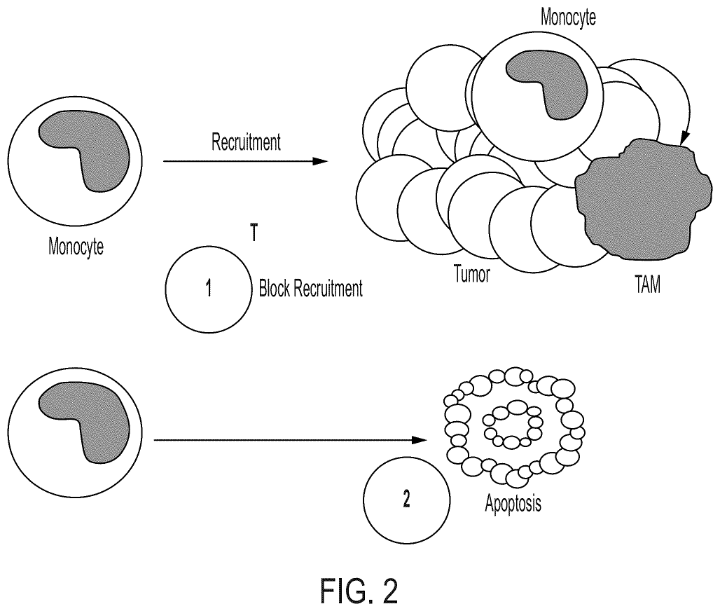

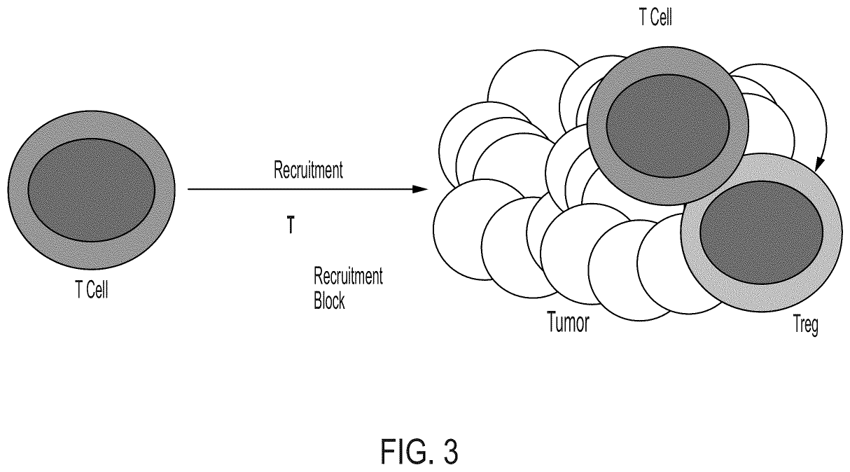

Monocytes and myeloid derived cells are recruited to the tumors in response to diverse chemoattractants including CSF1 and complement components. Inside tumors, monocytes and macrophages are predominantly of the M2 phenotype (tumor associated macrophages, TAMs). (FIG. 2) M2 cell types are activated by TH2 cytokines, e.g., IL-4 and IL-13.

In addition, endogenous myeloid cells, e.g. monocytes can compete with adoptively transferred myeloid cells for access to antigens and supportive cytokines. Provided herein a methods of preconditioning; that interfere with myeloid cell, specifically monocyte and/or macrophages, or lymphocytes to be recruited at the site of the tumor which otherwise contribute to the pool of TAMs or Tregs respectively, for rendering the host suitable for a myeloid cell therapy. Provided herein a methods of preconditioning that deplete the TME of cells including, for example TAMs for rendering the host suitable for a myeloid cell therapy. Pretreatment with myeloid cell depleting agents removes this competition, resulting in an increase in the level of endogenous cytokines.

Provided herein is a pharmaceutical composition for use in treating a disease, e.g., cancer, wherein prior to administering the pharmaceutical composition, the subject is preconditioned for the therapy by administering an immune cell inhibitory agent, wherein the immune cell inhibitory agent reduces the number of immune cells of the subject or inhibits a function of immune cells of the subject.

In one embodiment, the immune cell inhibitory agent is an agent, that upon application to a living system modulates one or more immune cells, or modulates a function of one or more immune cells. In some embodiments, the immune cell inhibitory agent upon application to a living system depletes one immune cell type in the system. In some embodiments, the immune cell inhibitory agent upon application to a living system depletes a particular immune cell type in the system. In some embodiments, the particular immune cell type are myeloid cells, e.g. monocytes, macrophages. In some embodiments, the particular immune cell type are lymphocyte cells, e.g. T cells. In some embodiments, “depletes” means “reduces” or “reduces considerably” or makes the living system devoid of, or nearly devoid of the cell type, for a period of time. In some embodiments, treating the host with an immune cell inhibitory agent reduces the number of a certain immune cell type in the host for a temporary period of time. In some embodiments, treating the host with an immune cell inhibitory agent reduces the number of a certain immune cell type in the host for a temporary period of time in the site of disease, e.g. at the site of tumor, or within the tumor, or within and around the tumor, for a temporary period. In some embodiments, treating the host with an immune cell inhibitory agent depletes the host of a certain immune cell type that the agent targets or is designed to target in the host for a temporary period of time; or depletes the site of the disease, e.g. the tissue or the organ harboring the diseased site, e.g. the tumor, or at the site of tumor, or within the tumor, or within and around the tumor of the certain immune cell type, for a temporary period. In some embodiments, the temporary period of time is the period within which the pharmaceutical composition comprising the population of cells comprising a therapeutically effective amount of monocytes comprising a recombinant polynucleic acid comprising a sequence encoding a chimeric fusion protein (CFP) is administered. In some embodiments, it is understood that the cellular homeostasis returns to the host system, or to the tissue or the organ harboring the disease site (e.g., the tumor), or the diseased site (e.g., the tumor)—naturally, at the end of the temporary period.

In some embodiments, the immune cell inhibitory agent reduces the number of the immune cell that the immune cell inhibitory agent targets or designed to target, by about at least 1.5 fold, at least 2 fold, at least 3 fold, at least 4 fold, at least 5 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, or at least 100 fold after the administration. In some embodiments, the immune cell inhibitory agent reduces the number of the immune cell that the immune cell inhibitory agent targets or designed to target, by greater than 100 fold.

In some embodiments, the immune cell inhibitory agent upon application to a living system alters at least one function of an immune cell. In some embodiments, the immune cell inhibitory agent upon application to a living system modulates at least one function of an immune cell. In some embodiments, the immune cell inhibitory agent alters (reduces) cell migration, reduces or prevents extravasation, reduces or prevents influx of the targeted immune cell at the site of the disease, e.g. the tumor or the tissue or organ or the locale comprising the tumor. In some embodiments, the immune cell inhibitory agent upon application to a living system alters cytokine secretion by the immune cell targeted by the immune cell inhibitory agent.

In some embodiments, the temporary period is 2 days, 3 days, 4 days, 5, days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days 44 days, 45 days, 46 days, 47 days, 48 days, 49 days, 50 days, 51 days, 52 days, 53 days, 54 days, 55 days, 56 days, 57 days, 58 days, 59 days or 60 days from the day of administering the immune cell inhibitory agent. In some embodiments, the temporary period is about 20 days, about 30 days, about 40 days, about 50 days, about 60 days, about 70 days, about 80 days, about 90 days, about 100 days, about 110 days, about 120 days, about 130 days, about 140 days, about 150 days, about 160 days, about 170 days, about 180 days, about 190 days, or about 200 days from the day of administering the immune cell inhibitory agent.

In some embodiments, the immune cell inhibitory agent is administered before, or during the administering of the pharmaceutical composition comprising a population of cells comprising a therapeutically effective amount of monocytes comprising a recombinant polynucleic acid, wherein the recombinant polynucleic acid comprises a sequence encoding a chimeric fusion protein (CFP).

In some embodiments, the preconditioning agent, e.g., the immune cell inhibitory agent or the immune cell modulating agent is administered at a time prior to administering the pharmaceutical composition comprising a population of cells comprising a therapeutically effective amount of monocytes comprising a recombinant polynucleic acid, wherein the recombinant polynucleic acid comprises a sequence encoding a chimeric fusion protein (CFP). In some embodiments, a pharmaceutical composition is provided herein, wherein the immune cell inhibitory agent has been administered or is administered before administering the pharmaceutical composition, such as, the immune cell inhibitory agent has been administered or is administered about 1, 2, 3, 4, 5, 6, 7, 8, 9, 11, 11, 12, 13, 14, 15, 16, 17 18, 19, 20, 21, 22, 23 or 24 hours before administering the pharmaceutical composition, or about 1, 2, 3, 4, 5, 6, 7, 8, 9, 11, 11, 12, 13 or 14 days before administering the pharmaceutical composition comprising a population of cells comprising a therapeutically effective amount of monocytes comprising a recombinant polynucleic acid, wherein the recombinant polynucleic acid comprises a sequence encoding a chimeric fusion protein (CFP).

In some embodiments, the immune cell inhibitory agent is administered at least 1 hour before administering the pharmaceutical composition. In some embodiments, the immune cell inhibitory agent is administered at least 2 hours before administering the pharmaceutical composition. In some embodiments, the immune cell inhibitory agent is administered at least 3 hours before administering the pharmaceutical composition. In some embodiments, the immune cell inhibitory agent is administered at least 4 hours before administering the pharmaceutical composition. In some embodiments, the immune cell inhibitory agent is administered at least 5 hours before administering the pharmaceutical composition. In some embodiments, the immune cell inhibitory agent is administered at least 6 hours before administering the pharmaceutical composition. In some embodiments, the immune cell inhibitory agent is administered at least 7 hours before administering the pharmaceutical composition. In some embodiments, the immune cell inhibitory agent is administered at least 8 hours before administering the pharmaceutical composition. In some embodiments, the immune cell inhibitory agent is administered at least 9 hours before administering the pharmaceutical composition. In some embodiments, the immune cell inhibitory agent is administered at least 10 hours before administering the pharmaceutical composition. In some embodiments, the immune cell inhibitory agent is administered at least 11 hours before administering the pharmaceutical composition. In some embodiments, the immune cell inhibitory agent is administered at least 12 hours before administering the pharmaceutical composition. In some embodiments, the immune cell inhibitory agent is administered at least 13 hours before administering the pharmaceutical composition. In some embodiments, the immune cell inhibitory agent is administered at least 14 hours before administering the pharmaceutical composition. In some embodiments, the immune cell inhibitory agent is administered at least 15 hours before administering the pharmaceutical composition. In some embodiments, the immune cell inhibitory agent is administered at least 16 hours before administering the pharmaceutical composition. In some embodiments, the immune cell inhibitory agent is administered at least 17 hours before administering the pharmaceutical composition. In some embodiments, the immune cell inhibitory agent is administered at least 18 hours before administering the pharmaceutical composition. In some embodiments, the immune cell inhibitory agent is administered at least 19 hours before administering the pharmaceutical composition. In some embodiments, the immune cell inhibitory agent is administered at least 20 hours before administering the pharmaceutical composition. In some embodiments, the immune cell inhibitory agent is administered at least 21 hours before administering the pharmaceutical composition. In some embodiments, the immune cell inhibitory agent is administered at least 22 hours before administering the pharmaceutical composition. In some embodiments, the immune cell inhibitory agent is administered at least 23 hours before administering the pharmaceutical composition. In some embodiments, the immune cell inhibitory agent is administered at least 24 hours before administering the pharmaceutical composition. In some embodiments, the immune cell inhibitory agent is administered at least 36 hours before administering the pharmaceutical composition. In some embodiments, the immune cell inhibitory agent is administered at 1 day before administering the pharmaceutical composition. In some embodiments, the immune cell inhibitory agent is administered at 2 days before administering the pharmaceutical composition. In some embodiments, the immune cell inhibitory agent is administered at 3 days before administering the pharmaceutical composition. In some embodiments, the immune cell inhibitory agent is administered at 4 days before administering the pharmaceutical composition. In some embodiments, the immune cell inhibitory agent is administered at 5 days before administering the pharmaceutical composition. In some embodiments, the immune cell inhibitory agent is administered at 6 days before administering the pharmaceutical composition. In some embodiments, the immune cell inhibitory agent is administered 7 days before administering the pharmaceutical composition. In some embodiments, the immune cell inhibitory agent is administered 8 days before administering the pharmaceutical composition. In some embodiments, the immune cell inhibitory agent is administered 9 days before administering the pharmaceutical composition. In some embodiments, the immune cell inhibitory agent is administered 10 days before administering the pharmaceutical composition. In some embodiments, the immune cell inhibitory agent is administered 11 days before administering the pharmaceutical composition. In some embodiments, the immune cell inhibitory agent is administered 12 days before administering the pharmaceutical composition. In some embodiments, the immune cell inhibitory agent is administered 13 days before administering the pharmaceutical composition. In some embodiments, the immune cell inhibitory agent is administered 14 days before administering the pharmaceutical composition. In some embodiments, the immune cell inhibitory agent is administered 15 days before administering the pharmaceutical composition. In some embodiments, the immune cell inhibitory agent is administered 16 days before administering the pharmaceutical composition. In some embodiments, the immune cell inhibitory agent is administered 17 days before administering the pharmaceutical composition. In some embodiments, the immune cell inhibitory agent is administered 18 days before administering the pharmaceutical composition. In some embodiments, the immune cell inhibitory agent is administered 19 days before administering the pharmaceutical composition. In some embodiments, the immune cell inhibitory agent is administered 20 days before administering the pharmaceutical composition. In some embodiments, the immune cell inhibitory agent is administered 21 days before administering the pharmaceutical composition. In some embodiments, the immune cell inhibitory agent is administered 22 days before administering the pharmaceutical composition. In some embodiments, the immune cell inhibitory agent is administered 23 days before administering the pharmaceutical composition. In some embodiments, the immune cell inhibitory agent is administered 24 days before administering the pharmaceutical composition. In some embodiments, the immune cell inhibitory agent is administered 25 days before administering the pharmaceutical composition. In some embodiments, the immune cell inhibitory agent is administered 26 days before administering the pharmaceutical composition. In some embodiments, the immune cell inhibitory agent is administered In some embodiments, the immune cell inhibitory agent is administered 27 days before administering the pharmaceutical composition. In some embodiments, the immune cell inhibitory agent is administered 28 days before administering the pharmaceutical composition. In some embodiments, the immune cell inhibitory agent is administered 29 days before administering the pharmaceutical composition. In some embodiments, 30 days before administering the pharmaceutical composition. In some embodiments, the immune cell inhibitory agent is administered 31 days before administering the pharmaceutical composition. In some embodiments, the immune cell inhibitory agent is administered 32 days before administering the pharmaceutical composition. In some embodiments, the immune cell inhibitory agent is administered 33 days before administering the pharmaceutical composition. In some embodiments, the immune cell inhibitory agent is administered 34 days before administering the pharmaceutical composition. In some embodiments, the immune cell inhibitory agent is administered 35 days before administering the pharmaceutical composition. In some embodiments, the immune cell inhibitory agent is administered 36 days before administering the pharmaceutical composition. In some embodiments, the immune cell inhibitory agent is administered 37 days before administering the pharmaceutical composition. In some embodiments, the immune cell inhibitory agent is administered 38 days before administering the pharmaceutical composition. In some embodiments, the immune cell inhibitory agent is administered 39 days before administering the pharmaceutical composition. In some embodiments, the immune cell inhibitory agent is administered 40 days before administering the pharmaceutical composition. In some embodiments, the immune cell inhibitory agent is administered 41 days before administering the pharmaceutical composition. In some embodiments, the immune cell inhibitory agent is administered 42 days before administering the pharmaceutical composition. In some embodiments, the immune cell inhibitory agent is administered 43 days In some embodiments, the immune cell inhibitory agent is administered In some embodiments, the immune cell inhibitory agent is administered 44 days before administering the pharmaceutical composition. In some embodiments, the immune cell inhibitory agent is administered 45 days before administering the pharmaceutical composition. In some embodiments, the immune cell inhibitory agent is administered 46 days before administering the pharmaceutical composition. In some embodiments, the immune cell inhibitory agent is administered 47 days before administering the pharmaceutical composition. In some embodiments, the immune cell inhibitory agent is administered 48 days before administering the pharmaceutical composition. In some embodiments, the immune cell inhibitory agent is administered 49 days before administering the pharmaceutical composition. In some embodiments, the immune cell inhibitory agent is administered 50 days before administering the pharmaceutical composition. In some embodiments, the immune cell inhibitory agent is administered 51 days before administering the pharmaceutical composition. In some embodiments, the immune cell inhibitory agent is administered 52 days before administering the pharmaceutical composition. In some embodiments, the immune cell inhibitory agent is administered 53 days before administering the pharmaceutical composition. In some embodiments, the immune cell inhibitory agent is administered 54 days before administering the pharmaceutical composition. In some embodiments, the immune cell inhibitory agent is administered 55 days before administering the pharmaceutical composition. In some embodiments, the immune cell inhibitory agent is administered 56 days before administering the pharmaceutical composition. In some embodiments, the immune cell inhibitory agent is administered 57 days before administering the pharmaceutical composition. In some embodiments, the immune cell inhibitory agent is administered 58 days before administering the pharmaceutical composition. In some embodiments, the immune cell inhibitory agent is administered 59 days or In some embodiments, the immune cell inhibitory agent is administered 60 days prior to the day of administering the pharmaceutical composition comprising a population of cells comprising a therapeutically effective amount of monocytes comprising a recombinant polynucleic acid, wherein the recombinant polynucleic acid comprises a sequence encoding a chimeric fusion protein (CFP).

In some embodiments, the pharmaceutical composition is administered to the human subject within about 1 hour from the time the human subject was administered the immune cell inhibitory agent. In some embodiments, the pharmaceutical composition is administered 2 hours from the time the human subject was administered the immune cell inhibitory agent. In some embodiments, the pharmaceutical composition is administered 3 hours from the time the human subject was administered the immune cell inhibitory agent. In some embodiments, the pharmaceutical composition is administered 4 hours from the time the human subject was administered the immune cell inhibitory agent. In some embodiments, the pharmaceutical composition is administered 5 hours from the time the human subject was administered the immune cell inhibitory agent. In some embodiments, the pharmaceutical composition is administered 6 hours from the time the human subject was administered the immune cell inhibitory agent. In some embodiments, the pharmaceutical composition is administered 7 hours from the time the human subject was administered the immune cell inhibitory agent. In some embodiments, the pharmaceutical composition is administered 8 hours from the time the human subject was administered the immune cell inhibitory agent. In some embodiments, the pharmaceutical composition is administered 9 hours from the time the human subject was administered the immune cell inhibitory agent. In some embodiments, the pharmaceutical composition is administered 10 hours from the time the human subject was administered the immune cell inhibitory agent. In some embodiments, the pharmaceutical composition is administered 11 hours from the time the human subject was administered the immune cell inhibitory agent. In some embodiments, the pharmaceutical composition is administered 12 hours from the time the human subject was administered the immune cell inhibitory agent. In some embodiments, the pharmaceutical composition is administered 13 hours from the time the human subject was administered the immune cell inhibitory agent. In some embodiments, the pharmaceutical composition is administered 14 hours from the time the human subject was administered the immune cell inhibitory agent. In some embodiments, the pharmaceutical composition is administered 15 hours from the time the human subject was administered the immune cell inhibitory agent. In some embodiments, the pharmaceutical composition is administered 16 hours from the time the human subject was administered the immune cell inhibitory agent. In some embodiments, the pharmaceutical composition is administered 17 hours from the time the human subject was administered the immune cell inhibitory agent. In some embodiments, the pharmaceutical composition is administered 18 hours from the time the human subject was administered the immune cell inhibitory agent. In some embodiments, the pharmaceutical composition is administered 19 hours from the time the human subject was administered the immune cell inhibitory agent. In some embodiments, the pharmaceutical composition is administered 20 hours from the time the human subject was administered the immune cell inhibitory agent. In some embodiments, the pharmaceutical composition is administered 21 hours from the time the human subject was administered the immune cell inhibitory agent. In some embodiments, the pharmaceutical composition is administered 22 hours from the time the human subject was administered the immune cell inhibitory agent. In some embodiments, the pharmaceutical composition is administered 23 hours from the time the human subject was administered the immune cell inhibitory agent. In some embodiments, the pharmaceutical composition is administered 24 hours from the time the human subject was administered the immune cell inhibitory agent. In some embodiments, the pharmaceutical composition is administered within about 1 day from the time the human subject was administered the immune cell inhibitory agent. In some embodiments, the pharmaceutical composition is administered within about 2 days from the time the human subject was administered the immune cell inhibitory agent. In some embodiments, the pharmaceutical composition is administered within about 3 days from the time the human subject was administered the immune cell inhibitory agent. In some embodiments, the pharmaceutical composition is administered within about 4 days from the time the human subject was administered the immune cell inhibitory agent. In some embodiments, the pharmaceutical composition is administered within about 5 days from the time the human subject was administered the immune cell inhibitory agent. In some embodiments, the pharmaceutical composition is administered within about 6 days from the time the human subject was administered the immune cell inhibitory agent. In some embodiments, the pharmaceutical composition is administered within about 7 days from the time the human subject was administered the immune cell inhibitory agent. In some embodiments, the pharmaceutical composition is administered within about 8 days from the time the human subject was administered the immune cell inhibitory agent. In some embodiments, the pharmaceutical composition is administered within about 9 days from the time the human subject was administered the immune cell inhibitory agent. In some embodiments, the pharmaceutical composition is administered within about 10 days from the time the human subject was administered the immune cell inhibitory agent. In some embodiments, the pharmaceutical composition is administered within about 11 days from the time the human subject was administered the immune cell inhibitory agent. In some embodiments, the pharmaceutical composition is administered within about 12 days from the time the human subject was administered the immune cell inhibitory agent. In some embodiments, the pharmaceutical composition is administered within about 13 days from the time the human subject was administered the immune cell inhibitory agent. 1 In some embodiments, the pharmaceutical composition is administered within about 4 days from the time the human subject was administered the immune cell inhibitory agent.

In some embodiments, the pharmaceutical composition is administered on the same day or at the same time as the immune cell inhibitory composition.

In some embodiments, the pharmaceutical composition comprising a population of cells comprising a therapeutically effective amount of monocytes comprising a recombinant polynucleic acid, wherein the recombinant polynucleic acid comprises a sequence encoding a chimeric fusion protein (CFP) is administered within less than 2 days, 3 days, 4 days, 5, days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days 44 days, 45 days, 46 days, 47 days, 48 days, 49 days, 50 days, 51 days, 52 days, 53 days, 54 days, 55 days, 56 days, 57 days, 58 days, 59 days or 60 days from the day of administering the immune cell inhibitory agent.

In some embodiments, the immune cell inhibitory agent is administered once.

In some embodiments, the immune cell inhibitory agent is administered more than once, e.g., twice, thrice, four times, five time, six times, seven times, eight times, nine times, ten times or more.

In one embodiments, the immune cell inhibitory agent is a small molecule, such as, a small molecule agonist, a small molecule antagonist, an inhibitor, an activator, a ligand. In some embodiments, the immune cell inhibitory agent is a polynucleotide or a polypeptide. In some embodiments, the immune cell inhibitory agent is a, siRNA, a morpholino product, a single stranded RNA, or an miRNA. In some embodiments the immune cell inhibitory agent is a CRISPR-CAS enabled gene editing system. In some embodiments, the immune cell inhibitor is an oligonucleotide or an oligopeptide. In some embodiments, the immune cell inhibitory agent is a peptide, an antibody or a fragment thereof. In some embodiments, the immune cell inhibitory agent is a neutralizing antibody or a fragment thereof. In some embodiment, the immune cell inhibitory agent is a blocking antibody or a fragment thereof. In some embodiment, the immune cell inhibitory agent is a stimulatory or agonistic antibody, or a fragment thereof. In some embodiment, the immune cell inhibitory agent is a cytokine inhibitor or blocker. In some embodiment, the immune cell inhibitory agent is a chemokine inhibitor. In some embodiment, the immune cell inhibitory agent is a complement activator. In some embodiment, the immune cell inhibitory agent is an apoptotic agent. In some embodiment, the immune cell inhibitory agent is cell-specific apoptotic agent. In some embodiment, the immune cell inhibitory agent is necrotic agent. In some embodiment, the immune cell inhibitory agent is cell-specific necrotic agent. In some embodiment, the immune cell inhibitory agent is pyroptotic agent. In some embodiment, the immune cell inhibitory agent is cell-specific pyroptotic agent. In some embodiment, the immune cell inhibitory agent comprises a cell-specific targeting moiety. In some embodiment, the immune cell inhibitory agent is a synthetic agent. In some embodiment, the immune cell inhibitory agent is a combination of one, two, three, four, five, six or more immune cell inhibitory agents. In some embodiment, the immune cell inhibitory agent is adjusted to be patient specific or subject specific. In some embodiment, the immune cell inhibitory agent is small molecule or a drug. In some embodiment, the immune cell inhibitory agent is an FDA approved drug. In some embodiment, the immune cell inhibitory agent is used in combination with one or more other drugs. In some embodiment, the immune cell inhibitory agent is adjusted for compatibility with one or more drugs or agents that are co-administered. In some embodiment, the immune cell inhibitory agent is designed to be cell-specific. In some embodiment, the immune cell inhibitory agent is a recombinant polynucleotide or recombinant polynucleotide product, e.g. a recombinant protein, polypeptide or a conjugated peptide.

TAM have strong immunosuppressive functions: they have been described to directly invalidate antitumor T-cell activity by suppressing CD8+ T-cell proliferation and IFNγ expression through programmed death ligand 1 (Mantovani et al, 2005; Kryczek et al, 2006; Kuang et al, 2009; Bloch et al, 2013). Notably, the response rates in the PD-1/PD-L1 trials relate, at least partially, to PD-L1 expression in the stroma (Herbst et al, 2014; Tumeh et al, 2014; Zhu et al, 2014; Qu et al, 2016), consistent with a role for macrophages and/or other stromal cells in blocking antitumor T-cell responses. In one embodiment, the immune cell inhibitory agent is directed towards starving the tumor microenvironment of the recruitment and influx of myeloid cells that become supportive M2 macrophages in the TME. In one embodiment, the immune cell inhibitory agent is directed towards starving the tumor microenvironment of the recruitment and influx of myeloid cells for a time sufficient for the tumor to be effectively receptive to the therapeutic myeloid cells administered as the pharmaceutical composition. In one embodiment, the immune cell inhibitory agent is directed towards starving the tumor microenvironment of the recruitment and influx of myeloid cells for a time sufficient for the tumor to be effectively weakened by depletion of the supportive TAM cells, such that when the therapeutic myeloid cells are administered as the pharmaceutical composition, the latter have a higher advantageous therapeutic effect over a condition where no immune cell inhibitory agent is administered. In one embodiment, the immune cell inhibitory agent is directed towards starving the tumor microenvironment of the recruitment and influx of myeloid cells for a time sufficient for the myeloid cells to effectively lyse tumor cells and reduce tumor mass or obliterate the tumor when administered as the pharmaceutical composition.

Interference with Myeloid Cell Recruitment

In one embodiment, the preconditioning for myeloid cell therapy comprises inhibiting monocyte recruitment to tumor by inhibiting chemotaxis of myeloid cells, as depicted in strategy 1 of FIG. 2.

In one embodiment, the immune cell inhibitory agent inhibits and/or binds to CCL2, CCL3, CCL7, CCL19, CCL21, CCL24, CCL25, CXCL8, CXCL11, CXCL12, XCL2, CCL3L1, CCR2 or CXCR4. In one embodiment the immune cell inhibitory agent interferes with the cellular activation, release and/or paracrine action of the chemokine receptor CXCR4 and its ligand CXCL12, also known as stromal cell-derived factor 1 (SDF-1), that support migration, proliferation, and survival of cancer cells. In some embodiments, the immune cell inhibitory agent inhibits binding of CXCL12 to CXCR4. In some embodiments, the immune cell inhibitory agent that inhibits CXCR4 function is AMD3100, also known as Plerixafor or Mozobil (Genzyme Corp), an FDA approved CXCR4 antagonist. CXCR4 is a mobilizer of hematopoietic stem cells in combination with G-CSF. AMD3100 is the prototype of bis-tetraazamacrocycles (bicyclams), a class of highly potent HIV1 antagonists. In one embodiment the immune cell inhibitory agent AMD3465, a monocyclam analog of AMD3100, in which the second cyclam ring of AMD3100 was substituted by a pyridinylmethylene group. Similarly as AMD3100, AMD3465 interferes with the binding of CXCL12 to CXCR4, thereby preventing CXCL12 to trigger CXCR4 endocytosis and CXCR4-induced intracellular signaling as calcium mobilization and MAPK activation. In some embodiments, the immune cell inhibitory agent is CXCR4 antagonist POL5551. In some embodiments, one or more of these compounds are administered at a time prior to a time for administering the pharmaceutical composition. In some embodiments, one or more doses of any of these agents suitable for the subject for the condition is administered at a time prior to a time for administering the pharmaceutical composition such that chemotaxis and recruitment of myeloid cells to the tumor are temporarily inhibited or stalled.

In some embodiments, the immune cell inhibitory agent inhibits and/or binds to CSF1R or CSF1. In some embodiments, the immune cell inhibitory agent is a CSF1R small-molecule inhibitor, pexidartinib also designated as (PLX3397), or PLX108-01. In some embodiments, the CSF1R small-molecule inhibitor is ARRY-382. In some embodiments, the CSF1R small-molecule inhibitor is PLX7486. In some embodiments, the CSF1R small-molecule inhibitor is BLZ945. In some embodiments, the CSF1R small-molecule inhibitor is JNJ-41346527. In some embodiments, the CSF1R small-molecule inhibitor is emactuzumab. In some embodiments, the CSF1R small-molecule inhibitor is AMG821. In some embodiments, the CSF1R small-molecule inhibitor is IMC-CS4. In some embodiments, the CSF1R small-molecule inhibitor is cabiralizumab. In some embodiments, the CSF1R small-molecule inhibitor is MCS110. PD-0360324.

In some embodiments, the immune cell inhibitory agent may be selected from any of the agents listed in Table 1. In some embodiments, a specific dose for any one of the agents may be selected from that disclosed in any of the clinical trials indicated in the table.

TABLE 1
Exemplary CSF1/CSF1R inhibitors (partially reproduced
from table 1, Cannarile et al. Journal for
ImmunoTherapy of Cancer (2017) 5: 53).

| | | |
|---|---|---|
| Immune cell | | |
| inhibitory | | |
| agent/Compound | Target | Clinical Trial identifier |

Small Molecules

| | | |
|---|---|---|
| Pexidartinib | CSF1R (and | NCT02071940, NCT02975700, |
| (PLX3397, | cKIT, Flt3) | NCT01499043, NCT01349036, |
| PLX108-01) | | NCT01217229, NCT01349049, |
| | | NCT01004861. |
| PLX7486 | CSF1R (and Trk) | NCT01804530 |
| ARRY-382 | CSF1R | NCT01316822 |
| JNJ-40346527 | CSF1R | NCT01572519 |
| BLZ945 | CSF1R | NCT02829723 |

Monoclonal antibodies

| | | |
|---|---|---|
| Emactuzumab | CSF1R | NCT01494688 |
| (RG7155) | | |
| AMG820 | CSF1R | NCT01444404 |
| IMC-CS4 | CSFIR | NCT01346358, NCT02265536 |
| (LY3022855) | | |
| MCS110 | CSF-1 | NCT00757757 |
| Cabiralizumab | CSF1R | NCT02526017 |
| (FPA008) | | |
| PD-0360324 | CSF1R | NCT02554812 |

In some embodiments, any of the agents may be co-administered with each other in a combination. In some embodiments, any one or more of these agents may be administered in combination with another agent, for example PD1 inhibitor PDL1 inhibitor. In some embodiments, the PD1 inhibitor is PDR001. In some embodiments, one or more of these agent may be administered one or more doses of PDR001. In some embodiments, the PDL1 inhibitor is an anti-PDL1 antibody, e.g., Nivolumab or Pembrolizumab or Durvalumab or Atezolimumab or Avelumab. In some embodiments, one or more of these agent may be administered one or more doses of Nivolumab. In some embodiments, one or more of these agent may be administered one or more doses of Pembrolizumab. In some embodiments, one or more of these agents may be administered with a CD40 agonist, e.g. RG7876. In some embodiments, one or more of the agents may be administered in combination with an anti-CTL4 mAb (e.g., Tremelimumab).

In one embodiment, the immune cell inhibitory agent is a C—C chemokine ligand 2 (CM) inhibitor, CCL2 stimulates tumor growth, metastasis, and angiogenesis. In some embodiments, the immune cell inhibitory agent is a CCL2 inhibitor, Cariumab, a human IgG1κ anti-CCL2 mAb.

In some embodiments, the immune cell inhibitory agent may comprise a inhibitor for macrophage migration inhibitory factor 2 (MIF-2). Both MIF-1 and MIF-2 are released from activated monocytes/macrophages and signal through the surface receptor CD74, leading to recruitment of CD44 into a signaling complex and subsequently initiating the ERK1/2 mitogen-activated protein kinase pathway. In addition, MIF-1 exerts chemokine-like functions through interaction with the noncognate receptors CXCR2 and CXCR4, leading to immune cell recruitment. This function is mediated by a pseudo-(E)LR motif present in MIF-1 but absent in MIF-2. An exemplary MIF inhibitory is 4-(3-carboxyphenyl)-2,5-pyridinedicarboxylic acid (4-CPPC) [J. Biol Chem. 2019 Dec. 6; 294(49): 18522-18531.]

### Myeloid Cell Depletion or Reducing Myeloid Cell Number

In one embodiment, preconditioning of a host for a myeloid cell therapy may comprise administering an immune cell inhibitory agent for depletion of TAMs. TAMs have critical functions by promoting angiogenesis and metastasis, suppressing adaptive immunity and expressing growth factors and matrix proteases and transient suppression of macrophage activities may be beneficial as a preconditioning approach prior to administering a myeloid cell therapeutic. A temporary decrease of this macrophage-like cell lineage can be achieved by treatment with bisphosphonates (BPs). In some embodiments, the immune cell inhibitory agent induces apoptosis of immune cells. In some embodiments, the immune cell inhibitory agent may comprise an agent that induces macrophage cell death, and the immune cell inhibitory agent may be one or more Clodronate, Pamidronate, Ibandronate and Zoledronate for killing of macrophages. BPs are synthetic analogues of pyrophosphate in which the P—O—P bridge has been replaced by a non-hydrolysable P—C—P bond. The presence of a nitrogen atom in the R₂ side chain divides them into two groups with different intracellular mechanisms of action: non-amino bisphosphonates or first generation bisphosphonates and amino bisphosphonates, classified as bisphosphonates of second and third generation, in which the nitrogen atom is enclosed in a heterocyclic ring [Rodan G A, Reszka A A (2002) Bisphosphonate mechanism of action. Curr Mol Med 2(6): 571-577.] In some embodiments, liposome-encapsulated bisphosphonates may be used to target phagocytes, e.g., macrophages for targeted cell death. In some embodiments, other liposomes, beads, or nanoparticles coated with Chlodronates (e.g., Clodronate-loaded liposomes, such as Clodrolip) may be used to target macrophages for uptake by phagocytosis and death, induced by the Chlodronates. For specifically targeting macrophages, PBs or chlodronates, may be performed by packaging the compounds in cells or particles that are readily taken up by the macrophages, such as damaged cells, inert particulate matter, or apoptosing cells. On the other hand, specific targeting of macrophage in TME may be achieved by directly applying the compounds in the tumor, e.g., injecting directly in the tumor. In some embodiments, specific targeting of macrophage may be achieved conjugating with a ligands that target the receptors on the macrophages, such as MARCO, or pattern recognition molecules. In some embodiment, the immune cell inhibitory agent may comprise selected bisphosphonates encapsulated in autologous RBCs for a preconditioning step.

In some embodiments, an immune cell inhibitory agent activates a caspase. In some embodiments, the immune cell inhibitory agent activates caspase 8.

In some embodiments, the immune cell inhibitory agent is carlumab, clodronate, ibandronate, pamidronate or zoledronic acid.

In some embodiments, the immune cell inhibitory agent inhibits a surface molecule on a myeloid cell and induces cell death of the cell. For example, in some embodiments, the immune cell inhibitory agent inhibits and/or binds to CD33. CD33 is a 67 kd transmembrane cell surface glycoprotein receptor that is specific for the myeloid lineage. CD33 is a myeloid specific member of the sialic acid-binding receptor family and is expressed highly on myeloid progenitor cells but at much lower levels in differentiated cells. Human CD33 has two tyrosine residues in its cytoplasmic domain (Y340 and Y3:58). When phosphorylated, these tyrosines could function as docking sites for the phosphatases, SHP-1 and/or SHP-2, enabling CD33 to function as an inhibitory receptor. Here we demonstrate that CD33 is tyrosine phosphorylated in the presence of the phosphatase inhibitor, pervanadate, and recruits SHP-1 and SHP-2. In some embodiments, the immune cell inhibitor may comprise an anti-CD33 antibody, such as Vadastuxinuirnab. Vadastuximab talirine (SGN-CD33A, 33A) is an antibody-drug conjugate consisting of pyrrolobenzodiazepine dimers linked to a monoclonal antibody targeting CD33. In some embodiments, the immune cell inhibitor may comprise an anti-CD33 antibody, gemtuzumab. CD33×CD64 hi specific antibodies have been used to enhance the lysis of leukemic cells by cytokine-activated monocytes. In some embodiments, the immune cell inhibitory agent may comprise a CD33×CD64 bispecific antibody.

In some embodiments, the immune cell inhibitory agent inhibits or reduces the expression of TREM-1 or TREM-2. In some embodiments, the immune cell inhibitory agent is an inhibitory peptide that blocks TREM-1. An exemplary TREM-1 inhibitory peptide may be LP17 (LQVIDSGLYRCVIYHPP) [Feng. et al., Frontiers in Neuroscience, 2019, Vol. 13, Article 769]. In some embodiments, the immune cell inhibitory agent is an anti-TREM-2 antibody, PY314.

### Preconditioning to Weaken the Tumor or TME

One or more immune cell inhibitory agent may contribute to reprogramming of TAMS or the TME. Additionally, in tissues, in response to diverse signals, cells of the monocyte-macrophage lineage undergo diverse forms of functional reprogramming. In neoplastic tissues the signals orchestrating macrophage function are diverse and differ considerably in different tumors or different parts of the same tumor, with different phenotypes. Drugs affecting the TME, for example, anti-angiogenic drugs, immune checkpoints inhibitors and, more recently, drugs targeting macrophages, such as kinase inhibitors or antibodies directed to the CSF-1 receptor (Zeisberger et al, 2006; Priceman et al, 2010; DeNardo et al, 201 Hume and MacDonald, 2011; Pyonteck et al, 2013; Ries et al, 2014) may be used as a preconditioning agent prior to administering a myeloid cell therapeutic.

In some embodiment, the immune cell inhibitory agent may comprise an agent or component that is a Tie-2 inhibitor. In some embodiment, the immune cell inhibitory agent may comprise an agent or component that is a CD40 agonist. In some embodiment, the immune cell inhibitory agent may comprise an agent or component that is a PD1/PDL1 inhibitor as exemplified above. In some embodiment, the immune cell inhibitor); agent may comprise an agent or component that is a CCR5/CCL5 inhibitor. In some embodiment, the immune cell inhibitory agent may comprise an agent or component for targeting MARCO, thereby specifically targeting macrophages, and leaving other cells unaffected. In some embodiment, the immune cell inhibitory agent may comprise an agent or component for specifically targeting PI3 μg/HDAC class IIa targeting. In some embodiments, the immune cell inhibitor is an immune cell modulator selected from the group consisting of a TLR-agonist, a DICER inhibitor, an HDAC inhibitor, a PI3-Kinase inhibitor and a myeloid cell surface binding agent.

In some embodiments, the immune cell inhibitory agent may be an inhibitor of poly ADP ribose polymerase (PARP). PARP possesses enzymatic ability to synthesize and attach poly (ADP-ribose) (also known as PAR) to different protein substrates by a post-translational modification. PARP inhibitors act as antitumor agents. An exemplary PARP inhibitor is olaparib.

In some embodiments, the immune cell inhibitory agent is trabectedin or lurbinectedin. Trabectedin (ET743) is an anti-cancer drug that directly perturb the DNA metabolism. Lurbinectedin (PM01183) is a derivative of Trabectedin. Both drugs were shown to induce degradation of the RNA polymerase II (RNAPII) through the ubiquitin-proteasome pathway, and is shown to inhibit the transcription of selected cytokines (e.g., CCL2, IL6, IL8, PTX3) by TAMs abrogating their protumoral properties and modifying the tumor microenvironment. In tumor cells, lurbinectedin inhibits active transcription through the following: (I) its binding to CG-rich sequences, mainly located around promoters of protein coding genes; (2) the irreversible stalling of elongating RNA polymerase II on the DNA template and its specific degradation by the ubiquitin/proteasome machinery; and (3) the generation of XPF-dependent single-strand and double-strand DNA breaks, and subsequent apoptosis (Elez et al, 2014; Moneo et al, 2014; Pernice et al, 2016; Santamaria Nunez et al, 2016). In addition, lurbinectedin is extremely effective against cancer cells with impairment of homologous recombination repair (Romano et al, 2013). In some embodiments, Trabectedin may induce TRAIL and Caspase 8 pathway, and apoptosis of TAMs [Cassetta L et al Nat Rev Drug Discovery 2018]. In some embodiments, the immune cell inhibitory agent is an HDAC inhibitor. In some embodiments, the immune cell inhibitory agent comprises an anticancer drug, romidepsin. Romidepsin (FK228 or FR901228) is a cyclic depsipeptide small molecule that inhibits class I histone deacetylases. It is an FDA-approved drug for treatment of cutaneous and peripheral T-cell lymphoma. Romidepsin mediated inhibition of HDAC may result in cell cycle arrest and apoptosis of cancer cells. In some embodiments, the immune cell inhibitory agent comprises a VEGF inhibitor. TAMs are important mediators of the angiogenic switch in tumors and produce growth factors and other molecules which promote the vessel network, therefore, interfering with anti-angiogenic drugs. In some embodiments, the immune cell inhibitory agent comprising a VEGF inhibitor is bevacizumab. In some embodiments, the immune cell inhibitory agent is anti-VEGFR2 antibody ramucirumab.

In some embodiments, the immune cell inhibitory agent is a small molecule inhibitor of VEGF receptors VEGFR1/2/3, (these agents also block PDGFR-β, cKit, BRAF, FLT3 and CSF1R among other receptor tyrosine kinases) including sunitinib, axitinib, or sorafenib. In some embodiments, the immune cell inhibitory agent comprises a suitable dose of Avelumab and/or Bevacizumab. In some embodiments, the immune cell inhibitory agent comprises a suitable dose of Atezolizumab and/or Bevacizumab. In some embodiments, the immune cell inhibitory agent comprises a suitable dose of Nivolumab and/or Bevacizumab. In some embodiments, the immune cell inhibitory agent comprises a suitable dose of Ramucirumab and/or Paclitaxel. In some embodiments, the immune cell inhibitory agent comprises a suitable dose of Cabozantinib and/or Ipilumab. In some embodiments, the immune cell inhibitory agent comprises a suitable dose of Pembrolizumab and/or axitinib.

In some embodiments, the immune cell modulator is a IL-10, TGF-b, IL-4, an anti-CD41 agent, an anti-PD1 agent or an arginase inhibitor.

In some embodiments, cell therapy the preconditioning agent or the immune cell inhibitory agent is a myeloid and/or stromal checkpoint inhibitor. In some embodiments, preconditioning agent or the immune cell inhibitory agent is a monoclonal antibody that binds to and inhibits the inhibitory receptor LAIR1.

### Pharmaceutical Compositions for Myeloid Cell Therapy

### Engineered Myeloid Cells

Myeloid cells, including macrophages, are cells derived from the myeloid lineage and belong to the innate immune system. They are derived from bone marrow stem cells which egress into the blood and can migrate into tissues. Some of their main functions include phagocytosis, the activation of T cell responses, and clearance of cellular debris and extracellular matrices. Another hallmark function of certain phagocytic myeloid cells, such as monocytes, macrophages and dendritic cells is antigen presentation. After phagocytosis, the phagocytosed component is processed intracellularly, and an the myeloid cell presents the antigens on the surface of the cell in conjunction of an MHC molecule and associated helper molecules e.g., co-stimulatory molecules that are recognized by T cells in the milieu, by which T cells are activated which sets off the cascade of antigen-specific immune response and generation of immunological memory. They also play an important role in maintaining homeostasis, and initiating and resolving inflammation.

Myeloid cells can differentiate into numerous downstream cells, including macrophages, which can display different responses ranging from pro-inflammatory to anti-inflammatory depending on the type of stimuli they receive from the surrounding microenvironment. Furthermore, tissue macrophages have been shown to play a broad regulatory and activating role on other immune cell types including effector T cells, NK cells and T regulatory cells. Macrophages have been shown to be a main immune infiltrate in inflamed tissue and may, in some cases display and immune activating influence, or, in some cases may have a broad immunosuppressive influence on the tissue.

Myeloid cells are a major cellular compartment of the immune system comprising monocytes, dendritic cells, tissue macrophages, and granulocytes. Models of cellular ontogeny, activation, differentiation, and tissue-specific functions of myeloid cells have been revisited during the last years with surprising results. However, their enormous plasticity and heterogeneity, during both homeostasis and disease, are far from understood. Although myeloid cells have many functions, including phagocytosis and their ability to activate T cells, harnessing these functions for therapeutic uses has remained elusive. Newer avenues are therefore sought for using other cell types towards development of improved therapeutics, including but not limited to T cell malignancies.

A myeloid cell can refer broadly to cells of the myeloid lineage of the hematopoietic cell system, and can exclude, for example, the lymphocytic lineage. Myeloid cells comprise, for example, cells of the granulocyte lineage and monocyte lineages. Myeloid cells are differentiated from common progenitors derived from the hematopoietic stem cells in the bone marrow. Commitment to myeloid cell lineages may be governed by activation of distinct transcription factors, and accordingly myeloid cells may be characterized as cells having a level of plasticity, which may be described as the ability to further differentiate into terminal cell types based on extracellular and intracellular stimuli. Myeloid cells can be rapidly recruited into local tissues via various chemokine receptors on their surface. Myeloid cells are responsive to various cytokines and chemokines.

A myeloid cell, for example, may be a cell that originates in the bone marrow from a hematopoietic stem cell under the influence of one or more cytokines and chemokines, such as G-CSF, GM-CSF, Flt3L, CCL2, VEGF and S100A8/9. In some embodiments, the myeloid cell is a precursor cell. In some embodiments, the myeloid cell may be a cell having characteristics of a common myeloid progenitor, or a granulocyte progenitor, a myeloblast cell, or a monocyte-dendritic cell progenitor or a combination thereof. A myeloid can include a granulocyte or a monocyte or a precursor cell thereof. A myeloid can include an immature granulocyte, an immature monocyte, an immature macrophage, an immature neutrophil, and an immature dendritic cell.

A myeloid can include a monocyte or a pre-monocytic cell or a monocyte precursor. In some cases, a myeloid cell as used herein may refer to a monocyte having an M0 phenotype, an M1 phenotype or an M2 phenotype. A myeloid can include a dendritic cell (DC), a mature DC, a monocyte derived DC, a plasmacytoid DC, a pre-dendritic cell, or a precursor of a DC. A myeloid can include a neutrophil, which may be a mature neutrophil, a neutrophil precursor, or a polymorphonucleocyte (PMN). A myeloid can include a macrophage, a monocyte-derived macrophage, a tissue macrophage, a macrophage of an M0, an M1 or an M2 phenotype. A myeloid can include a tumor infiltrating monocyte (TIM). A myeloid can include a tumor associated monocyte (TAM). A myeloid can include a myeloid derived suppressor cell (MDSC). A myeloid can include a tissue resident macrophage. A myeloid can include a tumor associated DC (TADC). Accordingly, a myeloid cell may express one or more cell surface markers, for example, CD11b, CD14, CD15, CD16, CD38, CCR5, CD66, Lox-1, CD11c, CD64, CD68, CD163, CCR2, CCR5, HLA-DR, CD1c, CD83, CD141, CD209, MHC-II, CD123, CD303, CD304, a SIGLEC family protein and a CLEC family protein. In some cases, a myeloid cell may be characterized by a high or a low expression of one or more of cell surface markers, for example, CD11b, CD14, CD15, CD16, CD66, Lox-1, CD11c, CD64, CD68, CD163, CCR2, CCR5, HLA-DR, CD1c, CD83, CD141, CD209, MHC-II, CD123, CD303, CD304 or a combination thereof.

A myeloid cell may be involved in the process of phagocytosis. The process of phagocytosis can be closely coupled with an immune response and antigen presentation. The processing of exogenous antigens follows their uptake into professional antigen presenting cells by some type of endocytic event. Phagocytosis facilitate antigen presentation. For example, antigens from phagocytosed cells or pathogens, including cancer antigens, can be processed and presented on the cell surface of APCs.

Instant disclosure encompasses herein a population of human myeloid cells, particularly, for example, one or more various cells derived from the monocyte lineage, engineered to comprise an effective amount of a recombinant nucleic acid encoding a human autoimmune antigen. In one aspect, provided herein is a population of human monocytes comprising an effective amount a recombinant human autoimmune antigen.

Engineered myeloid cells can also be short-lived in vivo, phenotypically diverse, sensitive, plastic, and are often found to be difficult to manipulate in vitro. For example, engineered myeloid cells of the monocyte lineage in which a recombinant nucleic acid is incorporated, say for example, by transfection, or transduction, for example by a viral vector, is prone to alteration de novo, (where, by “alteration de novo” it is herein intended to convey that the alteration is independent of the identity or characteristics of the protein or polypeptide encoded by the nucleic acid, or its expression characteristics in the cell concerned), e.g., physiologically mature, differentiate, become terminally differentiated, lose plasticity, express one or more different cell surface marker, are activated differently, release one or more cytokines or chemokines distinct from its state prior to the transfection or transduction, exhibit altered phagocytic property, or even initiate cell death of the myeloid cell. In one embodiment, the instant disclosure encompasses carefully directing engineered myeloid cells of the monocytic lineage toward a physiologically controlled fate for utilization of cell in a desired immunotherapy.

In one embodiment, the recombinant nucleic acid comprises a viral vector, DNA plasmid or an RNA vector. The myeloid cell is engineered to comprise an effective amount of a recombinant nucleic acid encoding a chimeric fusion protein. An effective amount of the recombinant nucleic acid encoding a chimeric fusion protein comprises an amount that is sufficient to express the polypeptide encoded by the recombinant nucleic acid, e.g., the human autoimmune antigen. In one embodiment, the effective amount of the recombinant nucleic acid is an amount corresponding to about 1-100 copy numbers of a polynucleotide encoding the chimeric fusion protein per engineered cell. In one embodiment, the effective amount of the recombinant nucleic acid is an amount corresponding to about 1-200 copy numbers of a polynucleotide encoding the chimeric fusion protein per engineered cell. In one embodiment, the effective amount of the recombinant nucleic acid is an amount corresponding to about 1-300 copy numbers of a polynucleotide encoding the chimeric fusion protein per engineered cell. In one embodiment, the effective amount of the recombinant nucleic acid is an amount corresponding to about 1-400 copy numbers of a polynucleotide encoding the chimeric fusion protein per engineered cell. In one embodiment, the effective amount of the recombinant nucleic acid is an amount corresponding to about 1-500 copy numbers of a polynucleotide encoding the chimeric fusion protein per engineered cell. In one embodiment, the effective amount of the recombinant nucleic acid is an amount corresponding to about 1-600 copy numbers of a polynucleotide encoding the chimeric fusion protein per engineered cell. In one embodiment, the effective amount of the recombinant nucleic acid is an amount corresponding to about 1-700 copy numbers of a polynucleotide encoding the chimeric fusion protein per engineered cell. In one embodiment, the effective amount of the recombinant nucleic acid is an amount corresponding to about 1-800 copy numbers of a polynucleotide encoding the chimeric fusion protein per engineered cell. In one embodiment, the effective amount of the recombinant nucleic acid is an amount corresponding to about 1-900 copy numbers of a polynucleotide encoding the chimeric fusion protein per engineered cell. In one embodiment, the effective amount of the recombinant nucleic acid is an amount corresponding to about 1-1000 copy numbers of the chimeric fusion protein per engineered cell. In some embodiments, the effective amount of the recombinant nucleic acid is an amount corresponding to 1 copy of the polynucleotide encoding the chimeric fusion protein per engineered cell. In some embodiments, the effective amount of the recombinant nucleic acid is an amount corresponding to 2, 3, 4, 5, 6, 7, 8, 9 or 10 copies of the polynucleotide encoding the chimeric fusion protein per engineered cell. In some embodiments, the effective amount of the recombinant nucleic acid is an amount corresponding to about 10, 12, 14, 16, 18, 20, about 30, about 40, about 50 copies, about 60 copies, about 70 copies, about 70 copies, about 80 copies, about 90 copies, or about 100 copies of the polynucleotide encoding the chimeric fusion protein per engineered cell. In some embodiments, the effective amount of the recombinant nucleic acid is an amount corresponding to about 200, 300, 400, 500, 600, 700, 800, 900 or about 1000 copies of the polynucleotide encoding the chimeric fusion protein per engineered cell. In some embodiments, the effective amount of the recombinant nucleic acid is an amount corresponding to greater than 1000 copies of the polynucleotide encoding the chimeric fusion protein per engineered cell.

In some embodiments, the effective amount of the recombinant nucleic acid is an amount corresponding to an amount that results in detectable expression of the chimeric fusion protein encoded by the engineered cell.

In some embodiments, the myeloid cell is transfected, e.g., electroporated with 1 microgram of recombinant polynucleotide encoding the chimeric fusion protein per 10\hat{ }6 cells in a 1 ml suspension of appropriate media. In some embodiments, the myeloid cell is transfected, e.g., electroporated with about 1 microgram to about 10 micrograms (e.g., 1 2, 3, 4, 5, 6, 7, 8, 9 or 10 micrograms) of recombinant polynucleotide encoding the chimeric fusion protein per 10\hat{ }6 cells in a 1 ml suspension of appropriate media. In some embodiments, the myeloid cell is transfected, e.g., electroporated with approximately about 1 microgram to about 100 micrograms (e.g., 1, 5, 10, 20, 30, 40, 50, 60, 70, 80, 90 or 100 micrograms) of recombinant polynucleotide encoding the chimeric fusion protein per 10\hat{ }6 cells in a 1 ml suspension of appropriate media.

In some embodiments, the engineered myeloid cells can be manipulated in vitro, such that the engineered myeloid cell expresses the chimeric fusion protein encoded by the recombinant polynucleotide after the recombinant polynucleotide is introduced into the myeloid cell, such that the autoimmune antigen is processed intracellularly and accumulates in the phagolysosomal vesicles. The engineered myeloid cells can be further manipulated in vitro such that the cell is an apoptotic cell that is thereafter phagocytosed by a phagocytic cell in vivo, once the engineered myeloid cells are introduced into a subject in need thereof, after which the phagocytic cell in turn presents the autoimmune antigen to T cells in vivo, resulting in reducing or ameliorating the autoimmune reaction. Provided herein is a population of apoptotic human monocytes comprising an effective amount of a recombinant chimeric fusion protein in one or more vesicles.

Provided herein are engineered myeloid cells (including, but not limited to, neutrophils, monocytes, myeloid dendritic cells (mDCs), mast cells and macrophages), designed to comprise a recombinant polynucleotide encoding one or more autoimmune antigen(s), where the engineered myeloid cells can be utilized for inducing tolerance against the one or more autoimmune antigen(s). In some embodiments, In some embodiments, the myeloid cell is a phagocytic and/or an antigen presenting cell. In some embodiments, the cell is a stem cell derived cell, a myeloid cell, a monocyte, a macrophage, a dendritic cell, a mast cell, a neutrophil, a microglia, or an astrocyte. In some embodiments, the cell is an M1 monocyte. In some embodiments, the cell is an M2 monocyte. In some embodiments, the cell is an M1 macrophage cell. In some embodiments, the cell is an M2 macrophage cell. In some embodiments, the cell is an M1 myeloid cell. In some embodiments, the cell is an M2 myeloid cell. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In some embodiments, the myeloid cells are CD14⁺CD16⁻ human monocytes.

In some embodiments, the myeloid cells are CD14^{dim}CD16⁺ human monocytes.

In some embodiments, the myeloid cells are CD14⁺CD16⁺ human monocytes.

In some embodiments, the myeloid cells are CD14⁻CD16⁻ human monocytes

In some embodiments, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a tRNA. In some embodiments, the recombinant nucleic acid is a microRNA. Also provided herein is a vector comprising a recombinant nucleic acid sequence encoding one or more autoantigens described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions. In some embodiments, the RNA vector comprises a 5′UTR from a highly expressed gene. In some embodiments, the RNA vector comprises a stabilizing 3′UTR. In some embodiments, the RNA vector comprises a stabilizing 3′UTR from B-globin. In some embodiments, the RNA vector comprises a triplex forming sequence. In some embodiments, the RNA vector comprises a MascRNA-tRNA like sequence. In some embodiments, the RNA vector comprises a flavivirus sfRNA.

Also provided herein is a polypeptide encoded by the recombinant nucleic acid of a composition described herein. Also provided herein is a pharmaceutical composition comprising a composition described herein, such as a recombinant nucleic acid described herein, a vector described herein, a polypeptide described herein or a cell described herein; and a pharmaceutically acceptable excipient.

In some embodiments, the human monocytes are elutriation-purified human monocytes.

In some embodiments, the human monocytes are derived from the human subject.

In some embodiments, at least 10\hat{ }8 to about 10\hat{ }12 PBMCs are needed, from which cells of interest are isolated (enriched). In some embodiments, the cells of interest are CD14+ cells. In some embodiments the cells of interest are CD14+/CD16− cells. In some embodiments, the cells of interest are CD14+/CD16− cells, that may express high levels of a cell surface protein, other than CD14 or CD16. In some embodiments the cells of interest may express high levels of CCR2. In some embodiments, total cells prior to isolation or enrichment of cells of interest may be about 10\hat{ }8, 5×10\hat{ }8, 10\hat{ }9, 5×10\hat{ }9, 10¹⁰, 5×10¹⁰, 10¹¹, 5×10¹¹, 10\hat{ }12, 5×10\hat{ }12 cells, or more. In some embodiments, the total number of PBMCs before isolation or enrichment of cells of interest may be at least 10\hat{ }9 to about 10\hat{ }12 cells. In some embodiments, total cells prior to isolation or enrichment of cells of interest may be about 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, or 10\hat{ }10 cells; about 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10 cells or 10\hat{ }11 cell; about 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, or 10\hat{ }12 cells; about 5×10\hat{ }12, or more.

In some embodiments, greater than at least 50% of the isolated cells may be CD14+ as determined by a suitable assay, such as a flow cytometry assay using an aliquot of the recovered cells. In some embodiments, greater than at least 60% of the isolated cells may be CD14+. In some embodiments, greater than at least 70% of the isolated cells may be CD14+. In some embodiments, greater than at least 71%, 72%, 73%, 74%, 75%, 76%, 77%, 78%, 79% or 80% of the isolated cells may be CD14+. In some embodiments, greater than 81%, 82%, 83%, 84%, 85%, 86%, 87%, 88%, 89%, or 90% of the isolated cells may be CD14+. In some embodiments, greater than 91% of the isolated cells may be CD14+. In some embodiments, greater than 92% of the isolated cells may be CD14+. In some embodiments, greater than 93% of the isolated cells may be CD14+. In some embodiments, greater than 94% of the isolated cells may be CD14+. In some embodiments, greater than 95% of the isolated cells may be CD14+. In some embodiments, greater than 96% of the isolated cells may be CD14+. In some embodiments, greater than 97% of the isolated cells may be CD14+. In some embodiments, greater than 98% of the isolated cells may be CD14+. In some embodiments, greater than 99% of the isolated cells may be CD14+.

Isolated cells may be CD16− as determined by a flow cytometry assay using an aliquot of the recovered cells. In some embodiments, at least 50% of the isolated cells may be CD16− as determined by a flow cytometry assay using an aliquot of the recovered cells. In some embodiments, at least 60% of the isolated cells may be CD16−. In some embodiments, at least 70% of the isolated cells may be CD16−. In some embodiments, greater than at least 71%, 72%, 73%, 74%, 75%, 76%, 77%, 78%, 79% or 80% of the isolated cells may be CD16−. In some embodiments, greater than 81%, 82%, 83%, 84%, 85%, 86%, 87%, 88%, 89%, or 90% of the isolated cells may be CD16−. In some embodiments, greater than 91% of the isolated cells may be CD16−. In some embodiments, greater than 92% of the isolated cells may be CD16−. In some embodiments, greater than 93% of the isolated cells may be CD16−. In some embodiments, greater than 94% of the isolated cells may be CD16−. In some embodiments, greater than 95% of the isolated cells may be CD16−. In some embodiments, greater than 96% of the isolated cells may be CD16−. In some embodiments, greater than 97% of the isolated cells may be CD16−. In some embodiments, greater than 98% of the isolated cells may be CD16−. In some embodiments, greater than 99% of the isolated cells may be CD16−.

In some embodiments, at least 50%, 55%, 60%, 65% or 70% of the isolated or enriched cells may be CD14+/CD16−. In some embodiments, at least 75% of the isolated cells or enriched may be CD14+/CD16−. In some embodiments, at least 80% of the isolated or enriched cells may be CD14+/CD16−. In some embodiments, at least 85% of the isolated or enriched cells may be CD14+/CD16−. In some embodiments, at least 90% of the isolated or enriched cells may be CD14+/CD16−. In some embodiments, at least 95% of the isolated or enriched cells may be CD14+/CD16−.

Isolated or enriched cells may comprise at least less than 5% CD3+ cells as determined by a flow cytometry assay using an aliquot of the recovered cells. Isolated cells may comprise at least less than 4% CD3+ cells. Isolated cells may comprise at least less than 3% CD3+ cells. Isolated cells may comprise at least less than 2% CD3+ cells. Isolated cells may comprise at least less than 5% CD19+ cells, as determined by a flow cytometry assay using an aliquot of the recovered cells. Isolated cells may comprise at least less than 4% CD19+ cells. Isolated cells may comprise at least less than 4% CD3+ cells. Isolated cells may comprise at least less than 3% CD19+ cells. Isolated cells may comprise at least less than 2% CD19+ cells. At least 5% of the isolated cells may be CD56− cells, as determined by a flow cytometry assay using an aliquot of the recovered cells. At least 4% of the isolated cells may be CD56− cells. At least 3% of the isolated cells may be CD56− cells. At least 2% of the isolated cells may be CD56− cells.

In some embodiments, less than 10% of the cells in the population of cells are dendritic cells. For example, the population of cells can comprise less than 9%, 8%, 7%, 6%, 5%, 4%, 3%, 2% or 1% dendritic cells. In some embodiments, at least 50% of the cells in the population of cells are CCR2+. For example, the population of cells can comprise at least 55%, 60%, 65%, 70%, 75%, 80%, 85%, 90% or 95% or more CCR2+ cells. In some embodiments, at least 50% of the cells in the population of cells are CCR5+. For example, the population of cells can comprise at least 55%, 60%, 65%, 70%, 75%, 80%, 85%, 90% or 95% or more CCR5+ cells. In some embodiments, at least 50% of the cells in the population of cells are CD11b+. For example, the population of cells can comprise at least 55%, 60%, 65%, 70%, 75%, 80%, 85%, 90% or 95% or more CD11b+ cells. In some embodiments, at least 50% of the cells in the population of cells are CD63+. For example, the population of cells can comprise at least 55%, 60%, 65%, 70%, 75%, 80%, 85%, 90% or 95% or more CD63+ cells. In some embodiments, at least 50% of the cells in the population of cells are CD16−. For example, the population of cells can comprise at least 55%, 60%, 65%, 70%, 75%, 80%, 85%, 90% or 95% or more CD16− cells. In some embodiments, at least 50% of the cells in the population of cells are CD56−. For example, the population of cells can comprise at least 55%, 60%, 65%, 70%, 75%, 80%, 85%, 90% or 95% or more CD56− cells. In some embodiments, at least 50% of the cells in the population of cells are CD3−. For example, the population of cells can comprise at least 55%, 60%, 65%, 70%, 75%, 80%, 85%, 90% or 95% or more CD3− cells. In some embodiments, at least 50% of the cells in the population of cells are CD19−. For example, the population of cells can comprise at least 55%, 60%, 65%, 70%, 75%, 80%, 85%, 90% or 95% or more CD19− cells. In some embodiments, at least 50% of the cells in the population of cells are CD42b−. For example, the population of cells can comprise at least 55%, 60%, 65%, 70%, 75%, 80%, 85%, 90% or 95% or more CD42b− cells. In some embodiments, less than 40% of the cells in the population of cells are macrophage cells. For example, the population of cells can comprise less than 35%, 30%, 25%, 20%, 15%, 10%, 9%, 8%, 7%, 6%, 5%, 4%, 3%, 2% or 1% or less macrophage cells.

In some embodiments, at least 25% of the cells in the population of cells are CD14+/CD16−/CD3−. For example, the population of cells can comprise at least 30%, 35%, 40%, 45%, 50%, 55%, 60%, 65%, 70%, 75%, 80%, 85%, 90% or 95% or more CD14+/CD16−/CD3− cells.

In some embodiments, at least 25% of the cells in the population of cells are CD14+/CD16−. For example, the population of cells can comprise at least 30%, 35%, 40%, 45%, 50%, 55%, 60%, 65%, 70%, 75%, 80%, 85%, 90% or 95% or more CD14+/CD16− cells.

In some embodiments, at least 25% of the cells in the population of cells are CD3−/CD19−/CD42b−/CD56−. For example, the population of cells can comprise at least 30%, 35%, 40%, 45%, 50%, 55%, 60%, 65%, 70%, 75%, 80%, 85%, 90% or 95% or more CD3−/CD19−/CD42b−/CD56− cells.

In some embodiments, at least 25% of the cells in the population of cells are CD14+/CD16−/CD11b+. For example, the population of cells can comprise at least 30%, 35%, 40%, 45%, 50%, 55%, 60%, 65%, 70%, 75%, 80%, 85%, 90% or 95% or more CD14+/CD16−/CD11b+ cells.

In some embodiments, at least 25% of the cells in the population of cells are CD14+/CD16−/CD11b+/CD3−/CD19−/CD42b−. For example, the population of cells can comprise at least 30%, 35%, 40%, 45%, 50%, 55%, 60%, 65%, 70%, 75%, 80%, 85%, 90% or 95% or more CD14+/CD16−/CD11b+/CD3−/CD19−/CD42b− cells.

In some embodiments, at least 25% of the cells in the population of cells are CD14+/CD16−/CD11b+/CD3−/CD19−/CD56−. For example, the population of cells can comprise at least 30%, 35%, 40%, 45%, 50%, 55%, 60%, 65%, 70%, 75%, 80%, 85%, 90% or 95% or more CD14+/CD16−/CD11b+/CD3−/CD19−/CD56− cells.

In some embodiments, at least 25% of the cells in the population of cells are CD14+/CD16−/CD11b+/CD3−/CD42b−/CD56−. For example, the population of cells can comprise at least 30%, 35%, 40%, 45%, 50%, 55%, 60%, 65%, 70%, 75%, 80%, 85%, 90% or 95% or more CD14+/CD16−/CD11b+/CD3−/CD42b−/CD56− cells.

In some embodiments, at least 25% of the cells in the population of cells are CD14+/CD16−/CD11b+/CD19−/CD42b−/CD56−. For example, the population of cells can comprise at least 30%, 35%, 40%, 45%, 50%, 55%, 60%, 65%, 70%, 75%, 80%, 85%, 90% or 95% or more CD14+/CD16−/CD11b+/CD19−/CD42b−/CD56− cells.

In some embodiments, at least 25% of the cells in the population of cells are CD14+/CD16−/CD3−/CD19−/CD42b−/CD56−. For example, the population of cells can comprise at least 30%, 35%, 40%, 45%, 50%, 55%, 60%, 65%, 70%, 75%, 80%, 85%, 90% or 95% or more CD14+/CD16−/CD3-/CD19−/CD42b−/CD56− cells.

In some embodiments, at least 25% of the cells in the population of cells are CD14+/CD11b+/CD3−/CD19−/CD42b−/CD56−. For example, the population of cells can comprise at least 30%, 35%, 40%, 45%, 50%, 55%, 60%, 65%, 70%, 75%, 80%, 85%, 90% or 95% or more CD14+/CD11b+/CD3−/CD19−/CD42b−/CD56− cells.

In some embodiments, at least 25% of the cells in the population of cells are CD16−/CD11b+/CD3−/CD19−/CD42b−/CD56−. For example, the population of cells can comprise at least 30%, 35%, 40%, 45%, 50%, 55%, 60%, 65%, 70%, 75%, 80%, 85%, 90% or 95% or more CD16−/CD11b+/CD3−/CD19−/CD42b−/CD56− cells.

In some embodiments, at least 25% of the cells in the population of cells are CD14+/CD16−/CD11b+/CD3−/CD19−/CD42b−/CD56−. For example, the population of cells can comprise at least 30%, 35%, 40%, 45%, 50%, 55%, 60%, 65%, 70%, 75%, 80%, 85%, 90% or 95% or more CD14+/CD16−/CD11b+/CD3−/CD19−/CD42b−/CD56− cells.

Following isolation or enrichment cells may be further characterized by functional assays, such as phagocytosis assay, or chemotaxis assay. In some embodiments, cells having the above characteristics are further carried forward for developing into therapeutically effective myeloid cells. Cells may be frozen after isolation or enrichment or advanced into the next steps for preparation of a pharmaceutical composition. In some embodiments, the myeloid cell is not transformed or activated prior to administering to a subject in need thereof.

In some embodiments, the CD14+/CD16− cell population may be isolated from a biological sample, e.g., peripheral blood, e.g. from a leukapheresis sample by negative selection, and then manipulated (e.g., engineered) in vitro (i.e., ex vivo) to incorporate the nucleic acid encoding one or more recombinant proteins, such as a CFP protein. In some embodiments, engineering the myeloid cell comprises incorporation of an exogenous nucleic acid by transfection, or electroporation or nucleofection of the exogenous nucleic acid, e.g., a recombinant nucleic acid. In some embodiments, the incorporation of nucleic acid is performed by electroporation. Incorporation of nucleic acid results in an engineered cell that expresses the recombinant protein, e.g., the CFP. In some embodiments, the CD14+/CD16− cell population, expressing the recombinant protein is cultured for 2-20 hours to stabilize the cell in vitro and for recovery from incorporation of the foreign nucleic acid in the cell. In some embodiments, the cell population is isolated by a method described herein to obtain cells that are CD14+/CD16− from the peripheral blood, e.g., PBMC, and electroporated within 1 −10 hours. In some embodiments, a sample aliquoted from the isolated population is tested for viability and expression of cell surface molecules, such as CD14 expression, CD16 expression, CD11b expression, CD3 expression, CD19 expression, CD56 expression, CD42b expression, CD63 expression, CCR2 expression, CCR5 and/or CXCR1 expression In some embodiments, the cell population is electroporated within 1 hour following isolation or enrichment. In some embodiments, the cell population is electroporated within 2 hours, within 1-3 hours, within 2-4 hours, within 1-5 hours, 3-6 hours, less than 6 hours, less than 8 hours or less than 10 hours from the time of isolation or enrichment. In some embodiments, the electroporated cell population may be cultured in vitro for at the most 2, 3, 4, 5, 6, 7, 8, 9, 10, 11, 12, hours before either (i) administering to a subject, or (ii) freezing for future use. In some embodiments, the electroporated cell population may be cultured in vitro for less than 12 hours before either (i) administering to a subject, or (ii) freezing for future use. In some embodiments, the electroporated cell population may be cultured in vitro for less than 18 hours before either (i) administering to a subject, or (ii) freezing for future use. In some embodiments, the electroporated cell population may be cultured in vitro for less than 24 hours before either (i) administering to a subject, or (ii) freezing for future use. In some embodiments, the electroporated cell population may be cultured in vitro for 0-2 hours before either (i) administering to a subject, or (ii) freezing for future use. In some embodiments, at least 50% of the cell population that have been engineered and cultured ex vivo according to a method of the invention comprise CD14+ and CD16− cells, that also express the CFP. In some embodiments, the cell population that have been engineered and cultured ex vivo according to a method of the invention comprise greater than 50% cells that are CD14+ and CD16− cells, that also express the CFP, and that the cell population comprise less than 10% dendritic cells. In some embodiments, the cells that have been engineered and cultured ex vivo according to a method of the invention comprise greater than 70-90% cells are not differentiated into DC like or macrophage-like phenotype, or cells that have phenotypes of CD16+ or CD14− cells. In some embodiments, the cells retain further differentiation potential. In some embodiments, the cells are unpolarized into M1 or M2 phenotypes and retain the capability to be differentiated when administered in vivo.

### Nucleic Acid Compositions

Provided herein are compositions comprising a recombinant nucleic acid encoding a chimeric fusion protein (CFP), such as a phagocytic receptor (PR) fusion protein (PFP), a scavenger receptor (SR) fusion protein (SFP), an integrin receptor (IR) fusion protein (IFP) or a caspase-recruiting receptor (caspase-CAR) fusion protein. A CFP encoded by the recombinant nucleic acid can comprise an extracellular domain (ECD) comprising an antigen binding domain that binds to an antigen of a target cell. The extracellular domain can be fused to a hinge domain or an extracellular domain derived from a receptor, such as CD2, CD8, CD28, CD68, a phagocytic receptor, a scavenger receptor or an integrin receptor. The CFP encoded by the recombinant nucleic acid can further comprise a transmembrane domain, such as a transmembrane domain derived from CD2, CD8, CD28, CD68, a phagocytic receptor, a scavenger receptor or an integrin receptor. In some embodiments, a CFP encoded by the recombinant nucleic acid further comprises an intracellular domain comprising an intracellular signaling domain, such as an intracellular signaling domain derived from a phagocytic receptor, a scavenger receptor or an integrin receptor. For example, the intracellular domain can comprise one or more intracellular signaling domains derived from a phagocytic receptor, a scavenger receptor or an integrin receptor. For example, the intracellular domain can comprise one or more intracellular signaling domains that promote phagocytic activity, inflammatory response, nitric oxide production, integrin activation, enhanced effector cell migration (e.g., via chemokine receptor expression), antigen presentation, and/or enhanced cross presentation. In some embodiments, the CFP is a phagocytic receptor fusion protein (PFP). In some embodiments, the CFP is a phagocytic scavenger receptor fusion protein (PFP). In some embodiments, the CFP is an integrin receptor fusion protein (IFP). In some embodiments, the CFP is an inflammatory receptor fusion protein. In some embodiments, a CFP encoded by the recombinant nucleic acid further comprises an intracellular domain comprising a recruitment domain. For example, the intracellular domain can comprise one or more PI3K recruitment domains, caspase recruitment domains or caspase activation and recruitment domains (CARDs).

Provided herein is a composition comprising a recombinant nucleic acid encoding a CFP comprising a phagocytic or tethering receptor (PR) subunit (e.g., a phagocytic receptor fusion protein (PFP)) comprising: (i) a transmembrane domain, and (ii) an intracellular domain comprising a phagocytic receptor intracellular signaling domain; and an extracellular antigen binding domain specific to an antigen, e.g., an antigen of or presented on a target cell; wherein the transmembrane domain and the extracellular antigen binding domain are operatively linked such that antigen binding to the target by the extracellular antigen binding domain of the fused receptor activated in the intracellular signaling domain of the phagocytic receptor.

Provided herein is a composition comprising a recombinant nucleic acid sequence encoding a CFP comprising a phagocytic or tethering receptor (PR) subunit (e.g., a phagocytic receptor fusion protein (PFP)) comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein upon binding of the CFP to the antigen of the target cell, the killing or phagocytosis activity of a myeloid cell, such as a neutrophil, monocyte, myeloid dendritic cell (mDC), mast cell or macrophage expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP.

Provided herein is a composition comprising a recombinant nucleic acid sequence encoding a CFP comprising a phagocytic or tethering receptor (PR) subunit (e.g., a phagocytic receptor fusion protein (PFP)) comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein upon binding of the CFP to the antigen of the target cell, the killing or phagocytosis activity of a myeloid cell, such as a neutrophil, monocyte, myeloid dendritic cell (mDC), mast cell or macrophage expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

In one aspect, provided herein is a pharmaceutical composition comprising: (a) a myeloid cell, such as a neutrophil, monocyte, myeloid dendritic cell (mDC), mast cell or macrophage cell comprising a recombinant polynucleic acid, wherein the recombinant polynucleic acid comprises a sequence encoding a chimeric fusion protein (CFP), the CFP comprising: (i) an extracellular domain comprising an anti-CD5 binding domain, and (ii) a transmembrane domain operatively linked to the extracellular domain; and (b) a pharmaceutically acceptable carrier; wherein the myeloid cell expresses the CFP and exhibits at least a 1.1-fold increase in phagocytosis of a target cell expressing CD5 compared to a myeloid cell not expressing the CFP. In some embodiments, the CD5 binding domain is a CD5 binding protein that comprises an antigen binding fragment of an antibody, an Fab fragment, an scFv domain or an sdAb domain. In some embodiments, the CD5 binding domain comprises an scFv comprising: (i) a variable heavy chain (V_{H}) sequence of SEQ ID NO: 1 or with at least 90% sequence identity to SEQ ID NO: 1; and (ii) a variable light chain (V_{L}) sequence of SEQ ID NO: 2 or with at least 70%, 75%, 80%, 85%, 90%, 91%, 92%, 93%, 94%, 95%, 96%, 97%, 98% or 99% sequence identity to SEQ ID NO: 2. In some embodiments, the CD5 binding domain comprises an scFv comprising SEQ ID NO: 33 or with at least 70%, 75%, 80%, 85%, 90%, 91%, 92%, 93%, 94%, 95%, 96%, 97%, 98% or 99% sequence identity to SEQ ID NO: 33. In some embodiments, the HER2 binding domain comprises an scFv comprising: (i) a variable heavy chain (V_{H}) sequence of SEQ ID NO: 8 or with at least 70%, 75%, 80%, 85%, 90%, 91%, 92%, 93%, 94%, 95%, 96%, 97%, 98% or 99% sequence identity to SEQ ID NO: 8; and (ii) a variable light chain (V_{L}) sequence of SEQ ID NO: 9 or with at least 70%, 75%, 80%, 85%, 90%, 91%, 92%, 93%, 94%, 95%, 96%, 97%, 98% or 99% sequence identity to SEQ ID NO: 9. In some embodiments, the CD5 binding domain comprises an scFv comprising SEQ ID NO: 32 or with at least 70%, 75%, 80%, 85%, 90%, 91%, 92%, 93%, 94%, 95%, 96%, 97%, 98% or 99% sequence identity to SEQ ID NO: 32. In some embodiments, the CFP further comprises an intracellular domain, wherein the intracellular domain comprises one or more intracellular signaling domains, and wherein a wild-type protein comprising the intracellular domain does not comprise the extracellular domain.

In some embodiments, the extracellular domain further comprises a hinge domain derived from CD8, wherein the hinge domain is operatively linked to the transmembrane domain and the anti-CD5 binding domain. In some embodiments, the extracellular hinge domain comprises a sequence of SEQ ID NO: 7 or with at least 70%, 75%, 80%, 85%, 90%, 91%, 92%, 93%, 94%, 95%, 96%, 97%, 98% or 99% sequence identity to SEQ ID NO: 7.

In some embodiments, the CFP comprises an extracellular domain fused to a transmembrane domain of SEQ ID NO: 30 or with at least 70%, 75%, 80%, 85%, 90%, 91%, 92%, 93%, 94%, 95%, 96%, 97%, 98% or 99% sequence identity to SEQ ID NO: 30. In some embodiments, the CFP comprises an extracellular domain fused to a transmembrane domain of SEQ ID NO: 31 or with at least 70%, 75%, 80%, 85%, 90%, 91%, 92%, 93%, 94%, 95%, 96%, 97%, 98% or 99% sequence identity to SEQ ID NO: 31.

In some embodiments, the transmembrane domain comprises a CD8 transmembrane domain. In some embodiments, the transmembrane domain comprises a sequence of SEQ ID NO: 6 or 29 or with at least 70%, 75%, 80%, 85%, 90%, 91%, 92%, 93%, 94%, 95%, 96%, 97%, 98% or 99% sequence identity to SEQ ID NO: 6 or 29. In some embodiments, the transmembrane domain comprises a sequence of SEQ ID NO: 18 or with at least 70%, 75%, 80%, 85%, 90%, 91%, 92%, 93%, 94%, 95%, 96%, 97%, 98% or 99% sequence identity to SEQ ID NO: 18. In some embodiments, the transmembrane domain comprises a sequence of SEQ ID NO: 34 or with at least 70%, 75%, 80%, 85%, 90%, 91%, 92%, 93%, 94%, 95%, 96%, 97%, 98% or 99% sequence identity to SEQ ID NO: 34. In some embodiments, the transmembrane domain comprises a sequence of SEQ ID NO: 19 or with at least 70%, 75%, 80%, 85%, 90%, 91%, 92%, 93%, 94%, 95%, 96%, 97%, 98% or 99% sequence identity to SEQ ID NO: 19.

In some embodiments, the CFP comprises one or more intracellular signaling domains that comprise a phagocytic signaling domain. In some embodiments, the phagocytosis signaling domain comprises an intracellular signaling domain derived from a receptor other than Megf10, MerTk, FcRα, and Bai1. In some embodiments, the phagocytosis signaling domain comprises an intracellular signaling domain derived from a receptor other than Megf10, MerTk, an FcR, and Bai1. In some embodiments, the phagocytosis signaling domain comprises an intracellular signaling domain derived from a receptor other than CD3ζ. In some embodiments, the phagocytosis signaling domain comprises an intracellular signaling domain derived from FcRγ, FcRα or FGRε. In some embodiments, the phagocytosis signaling domain comprises an intracellular signaling domain derived from CD3ζ. In some embodiments, the CFP comprises an intracellular signaling domain of any one of SEQ ID NOs: 3, 20, 27 and 28 or with at least 70%, 75%, 80%, 85%, 90%, 91%, 92%, 93%, 94%, 95%, 96%, 97%, 98% or 99% sequence identity to any one of SEQ ID NOs: 3, 20, 27 and 28. In some embodiments, the one or more intracellular signaling domains further comprises a proinflammatory signaling domain. In some embodiments, the proinflammatory signaling domain comprises a PI3-kinase (PI3K) recruitment domain. In some embodiments, the proinflammatory signaling domain comprises a sequence of SEQ ID NO: 4 or with at least 70%, 75%, 80%, 85%, 90%, 91%, 92%, 93%, 94%, 95%, 96%, 97%, 98% or 99% sequence identity to SEQ ID NO: 4. In some embodiments, the proinflammatory signaling domain is derived from an intracellular signaling domain of CD40. In some embodiments, the proinflammatory signaling domain comprises a sequence of SEQ ID NO: 5 or with at least 70%, 75%, 80%, 85%, 90%, 91%, 92%, 93%, 94%, 95%, 96%, 97%, 98% or 99% sequence identity to SEQ ID NO: 5. In some embodiments, the CFP comprises an intracellular signaling domain of SEQ ID NO: 21 or with at least 70%, 75%, 80%, 85%, 90%, 91%, 92%, 93%, 94%, 95%, 96%, 97%, 98% or 99% sequence identity to SEQ ID NO: 21. In some embodiments, the CFP comprises an intracellular signaling domain of SEQ ID NO: 23 or with at least 70%, 75%, 80%, 85%, 90%, 91%, 92%, 93%, 94%, 95%, 96%, 97%, 98% or 99% sequence identity to SEQ ID NO: 23.

In some embodiments, the CFP comprises a sequence of SEQ ID NO: 14 or with at least 70%, 75%, 80%, 85%, 90%, 91%, 92%, 93%, 94%, 95%, 96%, 97%, 98% or 99% sequence identity to SEQ ID NO: 14. In some embodiments, the CFP comprises a sequence of SEQ ID NO: 15 or with at least 70%, 75%, 80%, 85%, 90%, 91%, 92%, 93%, 94%, 95%, 96%, 97%, 98% or 99% sequence identity to SEQ ID NO: 15. In some embodiments, the CFP comprises a sequence of SEQ ID NO: 16 or with at least 70%, 75%, 80%, 85%, 90%, 91%, 92%, 93%, 94%, 95%, 96%, 97%, 98% or 99% sequence identity to SEQ ID NO: 16. In some embodiments, the CFP comprises a sequence of SEQ ID NO: 24 or with at least 70%, 75%, 80%, 85%, 90%, 91%, 92%, 93%, 94%, 95%, 96%, 97%, 98% or 99% sequence identity to SEQ ID NO: 24. In some embodiments, the CFP comprises a sequence of SEQ ID NO:25 or with at least 70%, 75%, 80%, 85%, 70%, 75%, 80%, 85%, 90%, 91%, 92%, 93%, 94%, 95%, 96%, 97%, 98% or 99%, 91%, 92%, 93%, 94%, 95%, 96%, 97%, 98% or 99% sequence identity to SEQ ID NO: 25.

In some embodiments, the CFP comprises: (a) an extracellular domain comprising: (i) a scFv that specifically binds CD5, and (ii) a hinge domain derived from CD8; a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRα, FcRγ or FcRε, and (ii) a second intracellular signaling domain: (A) comprising a PI3K recruitment domain, or (B) derived from CD40. In some embodiments, the CFP comprises as an alternative (c) to the above: an intracellular domain comprising at least two intracellular signaling domains, wherein the at least two intracellular signaling domains comprise: (i) a first intracellular signaling domain derived from a phagocytic receptor intracellular domain, and (ii) a second intracellular signaling domain derived from a scavenger receptor phagocytic receptor intracellular domain comprising: (A) comprising a PI3K recruitment domain, or (B) derived from CD40. Exemplary scavenger receptors from which an intracellular signaling domain may be derived may be found in Table 2. In some embodiments, the CFP comprises and intracellular signaling domain derived from an intracellular signaling domain of an innate immune receptor.

In some embodiments, the recombinant polynucleic acid is an mRNA. In some embodiments, the recombinant polynucleic acid is a circRNA. In some embodiments, the recombinant polynucleic acid is a viral vector. In some embodiments, the recombinant polynucleic acid is delivered via a viral vector.

In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In one aspect, provided herein is a method of treating cancer in a human subject in need thereof comprising administering a pharmaceutical composition to the human subject, the pharmaceutical composition comprising: (a) a myeloid cell comprising a recombinant polynucleic acid sequence, wherein the polynucleic acid sequence comprises a sequence encoding a chimeric fusion protein (CFP), the CFP comprising: (i) an extracellular domain comprising an anti-CD5 binding domain, and (ii) a transmembrane domain operatively linked to the extracellular domain; and (b) a pharmaceutically acceptable carrier; wherein the myeloid cell expresses the CFP.

In some embodiments, upon binding of the CFP to CD5 expressed by a target cancer cell of the subject killing or phagocytosis activity of the myeloid cell is increased by greater than 20% compared to a myeloid cell not expressing the CFP. In some embodiments, growth of a tumor is inhibited in the human subject.

In some embodiments, the cancer is a CD5+ cancer. In some embodiments, the cancer is leukemia, T cell lymphoma, or B cell lymphoma.

In some embodiments, the anti-CD5 binding domain is a CD5 binding protein that comprises an antigen binding fragment of an antibody, an scFv domain, an Fab fragment, or an sdAb domain. In some embodiments, the anti-CD5 binding domain is a protein or fragment thereof that binds to CD5, such as a ligand of CD5 (e.g., a natural ligand of CD5).

In some embodiments, the CFP further comprises an intracellular domain, wherein the intracellular domain comprises one or more intracellular signaling domains, wherein the one or more intracellular signaling domains comprises a phagocytosis signaling domain and wherein a wild-type protein comprising the intracellular domain does not comprise the extracellular domain.

In some embodiments, the phagocytosis signaling domain comprises an intracellular signaling domain derived from a receptor other than Megf10, MerTk, FcRα and Bai1. In some embodiments, the phagocytosis signaling domain comprises an intracellular signaling domain derived from FcRγ, FcRα or FGRε.

In some embodiments, the one or more intracellular signaling domains further comprises a proinflammatory signaling domain. In some embodiments, the proinflammatory signaling domain comprises a PI3-kinase (PI3K) recruitment domain. In some embodiments, the transmembrane domain comprises a CD8 transmembrane domain. In some embodiments, the extracellular domain comprises a hinge domain derived from CD8, a hinge domain derived from CD28 or at least a portion of an extracellular domain from CD68.

In some embodiments, the CFP comprises: (a) an extracellular domain comprising: (i) a scFv that specifically binds CD5, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FcRε, and (ii) a second intracellular signaling domain that: (A) comprises a PI3K recruitment domain, or (B) is derived from CD40. In some embodiments, the recombinant nucleic acid is mRNA or circRNA. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In some embodiments, the method further comprises administering an additional therapeutic agent selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity, an agent that promotes sequestration of lymphocytes in primary and/or secondary lymphoid organs, an agent that increases concentration of naïve T cells and central memory T cells in secondary lymphoid organs, and any combination thereof.

In some embodiments, the myeloid cell further comprises: (a) an endogenous peptide or protein that dimerizes with the CFP, (b) a non-endogenous peptide or protein that dimerizes with the CFP; and/or (c) a second recombinant polynucleic acid sequence, wherein the second recombinant polynucleic acid sequence comprises a sequence encoding a peptide or protein that interacts with the CFP; wherein the dimerization or the interaction potentiates phagocytosis by the myeloid cell expressing the CFP as compared to a myeloid cell that does not express the CFP.

In some embodiments, the myeloid cell exhibits (i) an increase in effector activity, cross-presentation, respiratory burst, ROS production, iNOS production, inflammatory mediators, extracellular vesicle production, phosphatidylinositol 3,4,5-trisphosphate production, trogocytosis with the target cell expressing the antigen, resistance to CD47 mediated inhibition of phagocytosis, resistance to LILRB1 mediated inhibition of phagocytosis, or any combination thereof; and/or (ii) an increase in expression of a IL-1, IL3, IL-6, IL-10, IL-12, IL-13, IL-23, TNFα, a TNF family of cytokines, CCL2, CXCL9, CXCL10, CXCL11, IL-18, IL-23, IL-27, CSF, MCSF, GMCSF, IL-17, IP-10, RANTES, an interferon, MHC class I protein, MHC class II protein, CD40, CD48, CD58, CD80, CD86, CD112, CD155, a TRAIL/TNF Family death receptor, TGFβ, B7-DC, B7-H2, LIGHT, HVEM, TL1A, 41BBL, OX40L, GITRL, CD30L, TIM1, TIM4, SLAM, PDL1, an MMP (e.g., MMP2, MMP7 and MMP9) or any combination thereof.

In some embodiments, the intracellular signaling domain is derived from a phagocytic or tethering receptor or wherein the intracellular signaling domain comprises a phagocytosis activation domain. In some embodiments, the intracellular signaling domain is derived from a receptor other than a phagocytic receptor selected from Megf10, MerTk, FcR-alpha, or Bai1. In some embodiments, the intracellular signaling domain is derived from a protein, such as receptor (e.g., a phagocytic receptor), selected from the group consisting of TNFR1, MDAS, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor 1, CR1, CD35, CD3ζ, a complement receptor, CR3, CR4, Tim-1, Tim-4 and CD169. In some embodiments, the intracellular signaling domain comprises a pro-inflammatory signaling domain. In some embodiments, the intracellular signaling domain comprises a pro-inflammatory signaling domain that is not a PI3K recruitment domain.

In some embodiments, the intracellular signaling domain is derived from an ITAM domain containing receptor.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a phagocytic receptor other than a phagocytic receptor selected from Megf10, MerTk, FcRα, or Bai1.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

In some embodiments, the intracellular signaling domain is derived from a receptor, such as a phagocytic receptor, selected from the group consisting of TNFR1, MDAS, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor 1, CR1, CD35, CD3, CR3, CR4, Tim-1, Tim-4 and CD169. In some embodiments, the intracellular signaling domain comprises a pro-inflammatory signaling domain.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a receptor, such as a phagocytic receptor, selected from the group consisting of TNFR1, MDAS, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor 1, CR1, CD35, CD3, CR3, CR4, Tim-1, Tim-4 and CD169.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the intracellular signaling domain is derived from a phagocytic receptor other than a phagocytic receptor selected from Megf10, MerTk, FcRα, or Bai1. In some embodiments, the intracellular signaling domain comprises a pro-inflammatory signaling domain. In some embodiments, the intracellular signaling domain comprises a PI3K recruitment domain, such as a PI3K recruitment domain derived from CD19. In some embodiments, the intracellular signaling domain comprises a pro-inflammatory signaling domain that is not a PI3K recruitment domain.

Provided herein is a composition comprising a recombinant nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain comprises a pro-inflammatory signaling domain that is not a PI3K recruitment domain.

Provided herein is a composition of an engineered CFP, such as a phagocytic receptor fusion protein, that may be expressed in a cell, such as a myeloid cell, such as to generate an engineered myeloid cell that can target a target cell, such as a diseased cell.

A target cell is, for example, a cancer cell. In some embodiments, the engineered myeloid cell, after engulfment of a cancer cell may present a cancer antigen on its cell surface to activate a T cell. An “antigen” is a molecule capable of stimulating an immune response. Antigens recognized by T cells, whether helper T lymphocytes (T helper (TH) cells) or cytotoxic T lymphocytes (CTLs), are not recognized as intact proteins, but rather as small peptides that associate with MHC proteins (such as class I or class II MHC proteins) on the surface of cells. During the course of a naturally occurring immune response, antigens that are recognized in association with class II MHC molecules on antigen presenting cells (APCs) are acquired from outside the cell, internalized, and processed into small peptides that associate with the class II MHC molecules.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold,-fold, 17-fold, 18-fold, 19-fold, 20-fold, 25-fold, 30-fold, 40-fold, 50-fold, 75-fold, or 100-fold compared to a cell not expressing the CFP.

In some embodiments, the target cell expressing the antigen is a cancer cell. In some embodiments, the target cell expressing the antigen is at least 0.8 microns in diameter.

In some embodiments, a cell expressing the CFP exhibits an increase in phagocytosis of a target cell expressing the antigen compared to a cell not expressing the CFP. In some embodiments, a cell expressing the CFP exhibits at least a 1.1-fold increase in phagocytosis of a target cell expressing the antigen compared to a cell not expressing the CFP. In some embodiments, a cell expressing the CFP exhibits at least a 2-fold, 3-fold, 4-fold, 5-fold, 6-fold, 7-fold, 8-fold, 9-fold, 10-fold, 20-fold, 30-fold or 50-fold increase in phagocytosis of a target cell expressing the antigen compared to a cell not expressing the CFP. In some embodiments, a cell expressing the CFP exhibits an increase in production of a cytokine compared to a cell not expressing the CFP. In some embodiments, the cytokine is selected from the group consisting of IL-1, IL3, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, an interferon and combinations thereof. In some embodiments, a cell expressing the CFP exhibits an increase in effector activity compared to a cell not expressing the CFP.

In some embodiments, the chimeric fusion protein (CFP) comprises an extracellular domain (ECD) targeted to bind to CD5 (CD5 binding domain), for example, comprising a heavy chain variable region (VH) having an amino acid sequence as set forth in SEQ ID NO: 1. In some embodiments, the chimeric CFP comprises a CD5 binding heavy chain variable domain comprising an amino acid sequence that has at least 80%, 85%, 90%, 95%, 96%, 97%, 98%, 99% sequence identity to SEQ ID NO: 1. In some embodiments, the extracellular domain (ECD) targeted to bind to CD5 (CD5 binding domain) comprises a light chain variable domain (V_{L}) having an amino acid sequence as set forth in SEQ ID NO: 2. In some embodiments, the chimeric CFP comprises a CD5 binding light chain variable domain comprising an amino acid sequence that has at least 80%, 85%, 90%, 95%, 96%, 97%, 98%, 99% sequence identity to SEQ ID NO: 2.

In some embodiments, the CFP comprises an extracellular domain targeted to bind to HER2 (HER2 binding domain) having for example a heavy chain variable domain amino acid sequence as set forth in SEQ ID NO: 8 and a light chain variable domain amino acid sequence as set forth in SEQ ID NO: 9. In some embodiments, the CFP comprises a HER2 binding heavy chain variable domain comprising an amino acid sequence that has at least 80%, 85%, 90%, 95%, 96%, 97%, 98%, 99% sequence identity to SEQ ID NO: 8. In some embodiments, the CFP comprises a HER2 binding light chain variable domain comprising an amino acid sequence that has at least 80%, 85%, 90%, 95%, 96%, 97%, 98%, 99% sequence identity to SEQ ID NO: 9.

In some embodiments, the CFP comprises a hinge connecting the ECD to the transmembrane (TM). In some embodiments the hinge comprises the amino acid sequence of the hinge region of a CD8 receptor. In some embodiments, the CFP may comprise a hinge having the amino acid sequence set forth in SEQ ID NO: 7 (CD8a chain hinge domain). In some embodiments, the PFP hinge region comprises an amino acid sequence that is at least 80%, 85%, 90%, 95%, 96%, 97%, 98%, 99% sequence identity to SEQ ID NO: 7.

In some embodiments, the CFP comprises a CD8 transmembrane region, for example having an amino acid sequence set forth in SEQ ID NO: 6. In some embodiments, the CFP TM region comprises an amino acid sequence that is at least 80%, 85%, 90%, 95%, 96%, 97%, 98%, 99% sequence identity to SEQ ID NO: 6.

In some embodiments, the CFP comprises an intracellular domain having an FcR domain. In some embodiments, the CFP comprises an FcR domain intracellular domain comprises an amino acid sequence set forth in SEQ ID NO: 3, or at least a sequence having 80%, 85%, 90%, 95%, 96%, 97%, 98%, 99% sequence identity to SEQ ID NO: 3.

In some embodiments, the CFP comprises an intracellular domain having a PI3K recruitment domain. In some embodiments the PI3K recruitment domain comprises an amino sequence set forth in SEQ ID NO: 4. In some embodiments the PI3K recruitment domain comprises an amino acid sequence that has at least 80%, 85%, 90%, 95%, 96%, 97%, 98%, 99% sequence identity to SEQ ID NO: 4.

In some embodiments, the CFP comprises an intracellular domain having a CD40 intracellular domain. In some embodiments the CD40 ICD comprises an amino sequence set forth in SEQ ID NO: 5. In some embodiments the CD40 ICD comprises an amino acid sequence that has at least 80%, 85%, 90%, 95%, 96%, 97%, 98%, 99% sequence identity to SEQ ID NO: 5.

TABLE 2A
Sequences of chimeric PFPs and domains thereof

| SEQ ID | | |
|---|---|---|
| NO | PFP/Domain | Sequence |
| 1 | Anti-CD5 heavy | EIQLVQSGGGLVKPGGSVRISCAASGYTFTNYGMNWVR |
| | chain variable | QAPGKGLEWMGWINTHTGEPTYADSFKGRFTFSLDDSK |
| | domain | NTAYLQINSLRAEDTAVYFCTRRGYDWYFDVWGQGTTV |
| | | TV |
| 2 | Anti-CD5 light | DIQMTQSPSSLSASVGDRVTITCRASQDINSYLSWFQQ |
| | chain variable | KPGKAPKTLIYRANRLESGVPSRFSGSGSGTDYTLTIS |
| | domain | SLQYEDFGIYYCQQYDESPWTFGGGTKLEIK |
| 33 | Anti-CD5 scFv | EIQLVQSGGGLVKPGGSVRISCAASGYTFTNYGMNWVR |
| | | QAPGKGLEWMGWINTHTGEPTYADSFKGRFTFSLDDSK |
| | | NTAYLQINSLRAEDTAVYFCTRRGYDWYFDVWGQGTTV |
| | | TVSSGGGGSGGGGSGGGGSDIQMTQSPSSLSASVGDRV |
| | | TITCRASQDINSYLSWFQQKPGKAPKTLIYRANRLESG |
| | | VPSRFSGSGSGTDYTLTISSLQYEDFGIYYCQQYDESP |
| | | WTFGGGTKLEIK |
| 3 | FcRγ-chain | LYCRRLKIQVRKAAITSYEKSDGVYTGLSTRNQETYET |
| | intracellular | LKHEKPPQ |
| | signaling domain | |
| 20 | FcRγ-chain | LYCRLKIQVRKAAITSYEKSDGVYTGLSTRNQETYETL |
| | intracellular | KHEKPPQ |
| | signaling domain | |
| 27 | FcRγ-chain | RLKIQVRKAAITSYEKSDGVYTGLSTRNQETYETLKHE |
| | intracellular | KPPQ |
| | signaling domain | |
| 4 | PI3K recruitment | YEDMRGILYAAPQLRSIRGQPGPNHEEDADSYENM |
| | domain | |
| 5 | CD40 | KKVAKKPTNKAPHPKQEPQEINFPDDLPGSNTAAPVQE |
| | intracellular | TLHGCQPVTQEDGKESRISVQERQ |
| | domain | |
| 6 | CD8α chain | IYIWAPLAGTCGVLLLSLVIT |
| | transmembrane | |
| | domain | |
| 29 | CD8α chain | IYIWAPLAGTCGVLLLSLVITLYC |
| | transmembrane | |
| | domain | |
| 7 | CD8α chain hinge | ALSNSIMYFSHFVPVFLPAKPTTTPAPRPPTPAPTIAS |
| | domain | QPLSLRPEACRPAAGGAVHTRGLD |
| 8 | Anti-HER2 heavy | DIQMTQSPSSLSASVGDRVTITCRASQDVNTAVAWYQQ |
| | chain variable | KPGKAPKLLIYSASFLYSGVPSRFSGSRSGTDFTLTIS |
| | domain | SLQPEDFATYYCQQHYTTPPTFGQGTKVEIKRTGSTSG |
| | | SGKPGSGEGSEVQLVE |
| 9 | Anti-HER2 light | LVQPGGSLRLSCAASGFNIKDTYIHWVRQAPGKGLEWV |
| | chain variable | ARIYPTNGYTRYADSVKGRFTISADTSKNTAYLQMNSL |
| | domain | RAEDTAVYYCSRWGGDGFYAMDVWGQGTLVTV |
| 32 | Anti-HER2 scFv | DIQMTQSPSSLSASVGDRVTITCRASQDVNTAVAWYQQ |
| | | KPGKAPKLLIYSASFLYSGVPSRFSGSRSGTDFTLTIS |
| | | SLQPEDFATYYCQQHYTTPPTFGQGTKVEIKRTGSTSG |
| | | SGKPGSGEGSEVQLVESSGGGGSGGGGSGGGGSLVQPG |
| | | GSLRLSCAASGFNIKDTYIHWVRQAPGKGLEWVARIYP |
| | | TNGYTRYADSVKGRFTISADTSKNTAYLQMNSLRAEDT |
| | | AVYYCSRWGGDGFYAMDVWGQGTLVTV |
| 17 | GMCSF Signal | MWLQSLLLLGTVACSIS |
| | peptide | |
| 18 | CD28 | FWVLVVVGGVLACYSLLVTVAFIIFWV |
| | transmembrane | |
| | domain | |
| 34 | CD2 | IYLIIGICGGGSLLMVFVALLVFYIT |
| | Transmembrane | |
| | domain | |
| 19 | CD68 | ILLPLIIGLILLGLLALVLIAFCII |
| | transmembrane | |
| | domain | |
| 21 | TNFR1 | QRWKSKLYSIVCGKSTPEKEGELEGTTTKPLAPNPSFS |
| | intracellular | PTPGFTPTLGFSPVPSSTFTSSSTYTPGDCPNFAAPRR |
| | domain | EVAPPYQGADPILATALASDPIPNPLQKWEDSAHKPQS |
| | | LDTDDPATLYAVVENVPPLRWKEFVRRLGLSDHEIDRL |
| | | ELQNGRCLREAQYSMLATWRRRTPRREATLELLGRVLR |
| | | DMDLLGCLEDIEEALCGPAALPPAPSLLR |
| 22 | TNFR2 | PLCLQREAKVPHLPADKARGTQGPEQQHLLITAPSSSS |
| | intracellular | SSLESSASALDRRAPTRNQPQAPGVEASGAGEARASTG |
| | domain | SSDSSPGGHGTQVNVTCIVNVCSSSDHSSQCSSQASST |
| | | MGDTDSSPSESPKDEQVPFSKEECAFRSQLETPETLLG |
| | | STEEKPLPLGVPDAGMKPS |
| 23 | MDA5 | MSNGYSTDENFRYLISCFRARVKMYIQVEPVLDYLTFL |
| | intracellular | PAEVKEQIQRTVATSGNMQAVELLLSTLEKGVWHLGWT |
| | domain | REFVEALRRTGSPLAARYMNPELTDLPSPSFENAHDEY |
| | | LQLLNLLQPTLVDKLLVRDVLDKCMEEELLTIEDRNRI |
| | | AAAENNGNESGVRELLKRIVQKENWFSAFLNVLRQTGN |
| | | NELVQELTGSDCSESNAEIEN |
| 30 | CD8α chain hinge | ALSNSIMYFSHFVPVFLPAKPTTTPAPRPPTPAPTIAS |
| | domain + | QPLSLRPEACRPAAGGAVHTRGLDIYIWAPLAGTCGVL |
| | transmembrane | LLSLVITLYC |
| | domain | |
| 31 | CD8α chain hinge | ALSNSIMYFSHFVPVFLPAKPTTTPAPRPPTPAPTIAS |
| | domain + | QPLSLRPEACRPAAGGAVHTRGLDIYIWAPLAGTCGVL |
| | transmembrane | LLSLVIT |
| | domain | |
| 14 | CD5-FcRγ-PI3K | MWLQSLLLLGTVACSISEIQLVQSGGGLVKPGGSVRIS |
| | | CAASGYTFTNYGMNWVRQAPGKGLEWMGWINTHTGEPT |
| | | YADSFKGRFTFSLDDSKNTAYLQINSLRAEDTAVYFCT |
| | | RRGYDWYFDVWGQGTTVTVSSGGGGSGGGGSGGGGSDI |
| | | QMTQSPSSLSASVGDRVTITCRASQDINSYLSWFQQKP |
| | | GKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQP |
| | | LSLRPEACRPAAGGAVHTRGLDIYIWAPLAGTCGVLLL |
| | | SLVITLYCRRLKIQVRKAAITSYEKSDGVYTGLSTRNQ |
| | | ETYETLKHEKPPQGSGSYEDMRGILYAAPQLRSIRGQP |
| | | GPNHEEDADSYENM |
| 15 | HER2-FcRγ-PI3K | MWLQSLLLLGTVACSISDIQMTQSPSSLSASVGDRVTI |
| | | TCRASQDVNTAVAWYQQKPGKAPKLLIYSASFLYSGVP |
| | | SRFSGSRSGTDFTLTISSLQPEDFATYYCQQHYTTPPT |
| | | FGQGTKVEIKRTGSTSGSGKPGSGEGSEVQLVESGGGL |
| | | VQPGGSLRLSCAASGFNIKDTYIHWVRQAPGKGLEWVA |
| | | RIYPTNGYTRYADSVKGRFTISADTSKNTAYLQMNSLR |
| | | AEDTAVYYCSRWGGDGFYAMDVWGQGTLVTVSSSGGGG |
| | | SGALSNSIMYFSHFVPVFLPAKPTTTPAPRPPTPAPTI |
| | | ASQPLSLRPEACRPAAGGAVHTRGLDIYIWAPLAGTCG |
| | | VLLLSLVITLYCRRLKIQVRKAAITSYEKSDGVYTGLS |
| | | TRNQETYETLKHEKPPQGSGSYEDMRGILYAAPQLRSI |
| | | RGQPGPNHEEDADSYENM |
| 16 | CD5-FcRγ-CD40 | MWLQSLLLLGTVACSISEIQLVQSGGGLVKPGGSVRIS |
| | | CAASGYTFTNYGMNWVRQAPGKGLEWMGWINTHTGEPT |
| | | YADSFKGRFTFSLDDSKNTAYLQINSLRAEDTAVYFCT |
| | | RRGYDWYFDVWGQGTTVTVSSGGGGSGGGGSGGGGSDI |
| | | QMTQSPSSLSASVGDRVTITCRASQDINSYLSWFQQKP |
| | | GKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQP |
| | | LSLRPEACRPAAGGAVHTRGLDIYIWAPLAGTCGVLLL |
| | | SLVITLYCRLKIQVRKAAITSYEKSDGVYTGLSTRNQE |
| | | TYETLKHEKPPQKKVAKKPTNKAPHPKQEPQEINFPDD |
| | | LPGSNTAAPVQETLHGCQPVTQEDGKESRISVQERQ |
| 24 | CD5-FcRγ-MDA5 | MWLQSLLLLGTVACSISEIQLVQSGGGLVKPGGSVRIS |
| | | CAASGYTFTNYGMNWVRQAPGKGLEWMGWINTHTGEPT |
| | | YADSFKGRFTFSLDDSKNTAYLQINSLRAEDTAVYFCT |
| | | RRGYDWYFDVWGQGTTVTVSSGGGGSGGGGSGGGGSDI |
| | | QMTQSPSSLSASVGDRVTITCRASQDINSYLSWFQQKP |
| | | KGAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQP |
| | | LSLRPEACRPAAGGAVHTRGLDIYIWAPLAGTCGVLLL |
| | | SLVITLYCRLKIQVRKAAITSYEKSDGVYTGLSTRNQE |
| | | TYETLKHEKPPQGSGSMSNGYSTDENFRYLISCFRARV |
| | | KMYIQVEPVLDYLTFLPAEVKEQIQRTVATSGNMQAVE |
| | | LLLSTLEKGVWHLGWTREFVEALRRTGSPLAARYMNPE |
| | | LTDLPSPSFENAHDEYLQLLNLLQPTLVDKLLVRDVLD |
| | | KCMEEELLTIEDRNRIAAAENNGNESGVRELLKRIVQK |
| | | ENWFSAFLNVLRQTGNNELVQELTGSDCSESNAEIEN |
| 25 | CD5-FcRγ-TNFR1 | MWLQSLLLLGTVACSISEIQLVQSGGGLVKPGGSVRIS |
| | | CAASGYTFTNYGMNWVRQAPGKGLEWMGWINTHTGEPT |
| | | YADSFKGRFTFSLDDSKNTAYLQINSLRAEDTAVYFCT |
| | | RRGYDWYFDVWGQGTTVTVSSGGGGSGGGGSGGGGSDI |
| | | QMTQSPSSLSASVGDRVTITCRASQDINSYLSWFQQKP |
| | | GKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQP |
| | | LSLRPEACRPAAGGAVHTRGLDIYIWAPLAGTCGVLLL |
| | | SLVITLYCRLKIQVRKAAITSYEKSDGVYTGLSTRNQE |
| | | TYETLKHEKPPQGSGSQRWKSKLYSIVCGKSTPEKEGE |
| | | LEGTTTKPLAPNPSFSPTPGFTPTLGFSPVPSSTFTSS |
| | | STYTPGDCPNFAAPRREVAPPYQGADPILATALASDPI |
| | | PNPLQKWEDSAHKPQSLDTDDPATLYAVVENVPPLRWK |
| | | EFVRRLGLSDHEIDRLELQNGRCLREAQYSMLATWRRR |
| | | TPRREATLELLGRVLRDMDLLGCLEDIEEALCGPAALP |
| | | PAPSLLR |
| 26 | CD5-FcRγ-TNFR2 | MWLQSLLLLGTVACSISEIQLVQSGGGLVKPGGSVRIS |
| | | CAASGYTFTNYGMNWVRQAPGKGLEWMGWINTHTGEPT |
| | | YADSFKGRFTFSLDDSKNTAYLQINSLRAEDTAVYFCT |
| | | RRGYDWYFDVWGQGTTVTVSSGGGGSGGGGSGGGGSDI |
| | | QMTQSPSSLSASVGDRVTITCRASQDINSYLSWFQQKP |
| | | GKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQP |
| | | LSLRPEACRPAAGGAVHTRGLDIYIWAPLAGTCGVLLL |
| | | SLVITLYCRLKIQVRKAAITSYEKSDGVYTGLSTRNQE |
| | | TYETLKHEKPPQGSGSPLCLQREAKVPHLPADKARGTQ |
| | | GPEQQHLLITAPSSSSSSLESSASALDRRAPTRNQPQA |
| | | PGVEASGAGEARASTGSSDSSPGGHGTQVNVTCIVNVC |
| | | SSSDHSSQCSSQASSTMGDTDSSPSESPKDEQVPFSKE |
| | | ECAFRSQLETPETLLGSTEEKPLPLGVPDAGMKPS |

Table 2B shows exemplary sequences of chimeric fusion protein domains and/or fragments thereof that are meant to be non-limiting for the disclosure. Underlines denote the CDR sequences in sequential order of CDR1, CDR2 and CDR3 for the respective heavy and light chains in accordance to the Kabat numbering system.

TABLE 2B
Exemplary Chimeric Fusion Proteins and Receptor Domains

| CFP/Domain | Sequence |
|---|---|
| CD5-FcR-PI3K | MWLQSLLLLGTVACSISEIQLVQSGGGLVKPGGSVRISCAASGYTFT |
| | NYGMNWVRQAPGKGLEWMGWINTHTGEPTYADSFKGRFTFSLDDSKN |
| | TAYLQINSLRAEDTAVYFCTRRGYDWYFDVWGQGTTVTVSSGGGGSG |
| | GGGSGGGGSDIQMTQSPSSLSASVGDRVTITCRASQDINSYLSWFQQ |
| | KPGKAPKTLIYRANRLESGVPSRFSGSGSGTDYTLTISSLQYEDFGI |
| | YYCQQYDESPWTFGGGTKLEIKSGGGGSGALSNSIMYFSHFVPVFLP |
| | AKPTTTPAPRPPTPAPTIASQPLSLRPEACRPAAGGAVHTRGLDIYI |
| | WAPLAGTCGVLLLSLVITLYCRRLKIQVRKAAITSYEKSDGVYTGLS |
| | TRNQETYETLKHEKPPQGSGSYEDMRGILYAAPQLRSIRGQPGPNHE |
| | EDADSYENM |
| HER2-FcR-PI3K | MWLQSLLLLGTVACSISDIQMTQSPSSLSASVGDRVTITCRASQDVN |
| | TAVAWYQQKPGKAPKLLIYSASFLYSGVPSRFSGSRSGTDFTLTISS |
| | LQPEDFATYYCQQHYTTPPTFGQGTKVEIKRTGSTSGSGKPGSGEGS |
| | EVQLVESGGGLVQPGGSLRLSCAASGFNIKDTYIHWVRQAPGKGLEW |
| | VARIYPTNGYTRYADSVKGRFTISADTSKNTAYLQMNSLRAEDTAVY |
| | YCSRWGGDGFYAMDVWGQGTLVTVSSSGGGGSGALSNSIMYFSHFVP |
| | VFLPAKPTTTPAPRPPTPAPTIASQPLSLRPEACRPAAGGAVHTRGL |
| | DIYIWAPLAGTCGVLLLSLVITLYCRRLKIQVRKAAITSYEKSDGVY |
| | TGLSTRNQETYETLKHEKPPQGSGSYEDMRGILYAAPQLRSIRGQPG |
| | PNHEEDADSYENM |
| CD5-FcR-CD40 | MWLQSLLLLGTVACSISEIQLVQSGGGLVKPGGSVRISCAASGYTFT |
| | NYGMNWVRQAPGKGLEWMGWINTHTGEPTYADSFKGRFTFSLDDSKN |
| | TAYLQINSLRAEDTAVYFCTRRGYDWYFDVWGQGTTVTVSSGGGGSG |
| | GGGSGGGGSDIQMTQSPSSLSASVGDRVTITCRASQDINSYLSWFQQ |
| | KPGKAPKTLIYRANRLESGVPSRFSGSGSGTDYTLTISSLQYEDFGI |
| | YYCQQYDESPWTFGGGTKLEIKSGGGGSGALSNSIMYFSHFVPVFLP |
| | AKPTTTPAPRPPTPAPTIASQPLSLRPEACRPAAGGAVHTRGLDIYI |
| | WAPLAGTCGVLLLSLVITLYCRLKIQVRKAAITSYEKSDGVYTGLST |
| | RNQETYETLKHEKPPQKKVAKKPTNKAPHPKQEPQEINFPDDLPGSN |
| | TAAPVQETLHGCQPVTQEDGKESRISVQERQ |
| CD5-FcR-MDA5 | MWLQSLLLLGTVACSISEIQLVQSGGGLVKPGGSVRISCAASGYTFT |
| | NYGMNWVRQAPGKGLEWMGWINTHTGEPTYADSFKGRFTFSLDDSKN |
| | TAYLQINSLRAEDTAVYFCTRRGYDWYFDVWGQGTTVTVSSGGGGSG |
| | GGGSGGGGSDIQMTQSPSSLSASVGDRVTITCRASQDINSYLSWFQQ |
| | KPGKAPKTLIYRANRLESGVPSRFSGSGSGTDYTLTISSLQYEDFGI |
| | YYCQQYDESPWTFGGGTKLEIKSGGGGSGALSNSIMYFSHFVPVFLP |
| | AKPTTTPAPRPPTPAPTIASQPLSLRPEACRPAAGGAVHTRGLDIYI |
| | WAPLAGTCGVLLLSLVITLYCRLKIQVRKAAITSYEKSDGVYTGLST |
| | RNQETYETLKHEKPPQGSGSMSNGYSTDENFRYLISCFRARVKMYIQ |
| | VEPVLDYLTFLPAEVKEQIQRTVATSGNMQAVELLLSTLEKGVWHLG |
| | WTREFVEALRRTGSPLAARYMNPELTDLPSPSFENAHDEYLQLLNLL |
| | QPTLVDKLLVRDVLDKCMEEELLTIEDRNRIAAAENNGNESGVRELL |
| | KRIVQKENWFSAFLNVLRQTGNNELVQELTGSDCSESNAEIEN |
| CD5-FcR-TNFR1 | MWLQSLLLLGTVACSISEIQLVQSGGGLVKPGGSVRISCAASGYTFT |
| | NYGMNWVRQAPGKGLEWMGWINTHTGEPTYADSFKGRFTFSLDDSKN |
| | TAYLQINSLRAEDTAVYFCTRRGYDWYFDVWGQGTTVTVSSGGGGSG |
| | GGGSGGGGSDIQMTQSPSSLSASVGDRVTITCRASQDINSYLSWFQQ |
| | KPGKAPKTLIYRANRLESGVPSRFSGSGSGTDYTLTISSLQYEDFGI |
| | YYCQQYDESPWTFGGGTKLEIKSGGGGSGALSNSIMYFSHFVPVFLP |
| | AKPTTTPAPRPPTPAPTIASQPLSLRPEACRPAAGGAVHTRGLDIYI |
| | WAPLAGTCGVLLLSLVITLYCRLKIQVRKAAITSYEKSDGVYTGLST |
| | RNQETYETLKHEKPPQGSGSQRWKSKLYSIVCGKSTPEKEGELEGTT |
| | TKPLAPNPSFSPTPGFTPTLGFSPVPSSTFTSSSTYTPGDCPNFAAP |
| | RREVAPPYQGADPILATALASDPIPNPLQKWEDSAHKPQSLDTDDPA |
| | TLYAVVENVPPLRWKEFVRRLGLSDHEIDRLELQNGRCLREAQYSML |
| | ATWRRRTPRREATLELLGRVLRDMDLLGCLEDIEEALCGPAALPPAP |
| | SLLR |
| CD5-FcR-TNFR2 | MWLQSLLLLGTVACSISEIQLVQSGGGLVKPGGSVRISCAASGYTFT |
| | NYGMNWVRQAPGKGLEWMGWINTHTGEPTYADSFKGRFTFSLDDSKN |
| | TAYLQINSLRAEDTAVYFCTRRGYDWYFDVWGQGTTVTVSSGGGGSG |
| | GGGSGGGGSDIQMTQSPSSLSASVGDRVTITCRASQDINSYLSWFQQ |
| | KPGKAPKTLIYRANRLESGVPSRFSGSGSGTDYTLTISSLQYEDFGI |
| | YYCQQYDESPWTFGGGTKLEIKSGGGGSGALSNSIMYFSHFVPVFLP |
| | AKPTTTPAPRPPTPAPTIASQPLSLRPEACRPAAGGAVHTRGLDIYI |
| | WAPLAGTCGVLLLSLVITLYCRLKIQVRKAAITSYEKSDGVYTGLST |
| | RNQETYETLKHEKPPQGSGSPLCLQREAKVPHLPADKARGTQGPEQQ |
| | HLLITAPSSSSSSLESSASALDRRAPTRNQPQAPGVEASGAGEARAS |
| | TGSSDSSPGGHGTQVNVTCIVNVCSSSDHSSQCSSQASSTMGDTDSS |
| | PSESPKDEQVPFSKEECAFRSQLETPETLLGSTEEKPLPLGVPDAGM |
| | KPS |
| GMCSF Signal | MWLQSLLLLGTVACSIS |
| peptide | |
| Anti-CD5 heavy | EIQLVQSGGGLVKPGGSVRISCAASGYTFTNYGMNWVRQAPGKGLEW |
| chain variable | MGWINTHTGEPTYADSFKGRFTFSLDDSKNTAYLQINSLRAEDTAVY |
| domain | FCTRRGYDWYFDVWGQGTTVTV |
| Anti-CD5 heavy | EIQLVQSGGGLVKPGGSVRISCAASGYTFTNYGMNWVRQAPGKGLEW |
| chain variable | MGWINTHTGEPTYADSFKGRFTFSLDDSKNTAYLQINSLRAEDTAVY |
| domain | FCTRRGYDWYFDVWGQGTTVTVSS |
| Anti-CD5 light | DIQMTQSPSSLSASVGDRVTITCRASQDINSYLSWFQQKPGKAPKTL |
| chain variable | IYRANRLESGVPSRFSGSGSGTDYTLTISSLQYEDFGIYYCQQYDES |
| domain | PWTFGGGTKLEIK |
| Anti-CD5 scFv | EIQLVQSGGGLVKPGGSVRISCAASGYTFTNYGMNWVRQAPGKGLEW |
| | MGWINTHTGEPTYADSFKGRFTFSLDDSKNTAYLQINSLRAEDTAVY |
| | FCTRRGYDWYFDVWGQGTTVTVSSGGGGSGGGGSGGGGSDIQMTQSP |
| | SSLSASVGDRVTITCRASQDINSYLSWFQQKPGKAPKTLIYRANRLE |
| | SGVPSRFSGSGSGTDYTLTISSLQYEDFGIYYCQQYDESPWTFGGGT |
| | KLEIK |
| Anti-CD5 scFv | MWLQSLLLLGTVACSISEIQLVQSGGGLVKPGGSVRISCAASGYTFT |
| with leader | NYGMNWVRQAPGKGLEWMGWINTHTGEPTYADSFKGRFTFSLDDSKN |
| sequence | TAYLQINSLRAEDTAVYFCTRRGYDWYFDVWGQGTTVTVSSGGGGSG |
| | GGGSGGGGSDIQMTQSPSSLSASVGDRVTITCRASQDINSYLSWFQQ |
| | KPGKAPKTLIYRANRLESGVPSRFSGSGSGTDYTLTISSLQYEDFGI |
| | YYCQQYDESPWTFGGGTKLEIK |
| Anti-HER2 light | DIQMTQSPSSLSASVGDRVTITCRASQDVNTAVAWYQQKPGKAPKLL |
| chain variable | IYSASFLYSGVPSRFSGSRSGTDFTLTISSLQPEDFATYYCQQHYTT |
| domain | PPTFGQGTKVEIKRTGSTSGSGKPGSGEGSEVQLVE |
| Anti-HER2 light | DIQMTQSPSSLSASVGDRVTITCRASQDVNTAVAWYQQKPGKAPKLL |
| chain variable | IYSASFLYSGVPSRFSGSRSGTDFTLTISSLQPEDFATYYCQQHYT |
| domain | TPPTFGQGTKVEIK |
| Anti-HER2 | LVQPGGSLRLSCAASGFNIKDTYIHWVRQAPGKGLEWVARIYPTNGY |
| heavy chain | TRYADSVKGRFTISADTSKNTAYLQMNSLRAEDTAVYYCSRWGGDGF |
| variable domain | YAMDVWGQGTLVTV |
| Anti-HER2 | EVQLVESGGGLVQPGGSLRLSCAASGFNIKDTYIHWVRQAPGKGLEW |
| heavy chain | VARIYPTNGYTRYADSVKGRFTISADTSKNTAYLQMNSLRAEDTAVY |
| variable domain | YCSRWGGDGFYAMDVWGQGTLVTVSS |
| Anti-HER2 scFv | DIQMTQSPSSLSASVGDRVTITCRASQDVNTAVAWYQQKPGKAPKLL |
| | IYSASFLYSGVPSRFSGSRSGTDFTLTISSLQPEDFATYYCQQHYTT |
| | PPTFGQGTKVEIKRTGSTSGSGKPGSGEGSEVQLVESSGGGGSGGGG |
| | SGGGGSLVQPGGSLRLSCAASGFNIKDTYIHWVRQAPGKGLEWVARI |
| | YPTNGYTRYADSVKGRFTISADTSKNTAYLQMNSLRAEDTAVYYCSR |
| | WGGDGFYAMDVWGQGTLVTV |
| Anti-HER2 scFv | DIQMTQSPSSLSASVGDRVTITCRASQDVNTAVAWYQQKPGKAPKLL |
| | IYSASFLYSGVPSRFSGSRSGTDFTLTISSLQPEDFATYYCQQHYTT |
| | PPTFGQGTKVEIKRTGSTSGSGKPGSGEGSEVQLVESGGGLVQPGGS |
| | LRLSCAASGFNIKDTYIHWVRQAPGKGLEWVARIYPTNGYTRYADSV |
| | KGRFTISADTSKNTAYLQMNSLRAEDTAVYYCSRWGGDGFYAMDVWG |
| | QGTLVTVSS |
| Anti-HER2 scFv | DIQMTQSPSSLSASVGDRVTITCRASQDVNTAVAWYQQKPGKAPKLL |
| | IYSASFLYSGVPSRFSGSRSGTDFTLTISSLQPEDFATYYCQQHYTT |
| | PPTFGQGTKVEIKRTGSTSGSGKPGSGEGSEVQLVESGGGLVQPGGS |
| | LRLSCAASGFNIKDTYIHWVRQAPGKGLEWVARIYPTNGYTRYADSV |
| | KGRFTISADTSKNTAYLQMNSLRAEDTAVYYCSRWGGDGFYAMDVWG |
| | QGTLVTVSS |
| Anti-CD137 | MEFGLSWLFLVAILKGVQCGLLDLRQGMFAQLVAQNVLLIDGPLSWY |
| binding domain | SDPGLAGVSLTGGLSYKEDTKELVVAKAGVYYVFFQLELRRVVAGEG |
| | SGSVSLALHLQPLRSAAGAAALALTVDLPPASSEARNSAFGFQGRLL |
| | HLSAGQRLGVHLHTEARARHAWQLTQGATVLGLFRVTPEIPAGLPSP |
| | RSE |
| Anti-CD70 | QVQLVQSGAEVKKPGASVKVSCKASGYTFTNYGMNWVRQAPGQGLEW |
| binding domain | MGWINTYTGEPTYADAFKGRVTMTTDTSTSTAYMELRSLRSDDTAVY |
| | YCARDYGDYGMDYWGQGTTVTVSSGSTSGSGKPGSSEGSTKGDIVMT |
| | QSPDSLAVSLGERATINCRASKSVSTSGYSFMHWYQQKPGQPPKLLI |
| | YLASNLESGVPDRFSGSGSGTDFTLTISSLQAEDVAVYYCQHSREVP |
| | WTFGQGTKVEIK |
| Anti- | QVQLQESGPGLVKPSQTLSLTCTVSGGSISSGYNWHWIRQPPGKGLE |
| Claudin18.2 | WIGYIHYTGSTNYNPALRSRVTISVDTSKNQFSLKLSSVTAADTAVY |
| binding domain | YCARIYNGNSFPYWGQGTTVTVSSGGGGSGGGGSGGGGSDIVMTQSP |
| (scFv) | DSLAYSLGERATINCKSSQSLFNSGNQKNYLTWYQQKPGQPPKLLIY |
| | WASTRESGVPDRFSGSGSGTDIFITISSLQAEDVAVYYCQNAYSFPY |
| | TFGGGTKLEIKR |
| Anti-Trop-2 | DIQLTQSPSSLSASVGDRVSITCKASQDVSIAVAWYQQKPGKAPKLL |
| binding domain | IYSASYRYTGVPDRFSGSGSGTDFTLTISSLQPEDFAVYYCQQHYIT |
| (scFv) | PLTFGAGTKVEIKRGGGGSGGGGSGGGGSQVQLQQSGSELKKPGASV |
| | KVSCKASGYTFTNYGMNWVKQAPGQGLKWMGWINTYTGEPTYTDDFK |
| | GRFAFSLDTSVSTAYLQISSLKADDTAVYFCARGGFGSSYWYFDVWG |
| | QGSLVTVSS |
| Anti-Trop-2 | QVQLQQSGSELKKPGASVKVSCKASGYTFTNYGMNWVKQAPGQGLKW |
| binding domain | MGWINTYTGEPTYTDDFKGRFAFSLDTSVSTAYLQISSLKADDTAVY |
| (scFv) | FCARGGFGSSYWYFDVWGQGSLVTVSSGGGGSGGGGSGGGGSDIQLT |
| | QSPSSLSASVGDRVSITCKASQDVSIAVAWYQQKPGKAPKLLIYSAS |
| | YRYTGVPDRFSGSGSGTDFTLTISSLQPEDFAVYYCQQHYITPLTFG |
| | AGTKVEIKR |
| Anti-Trop-2 VH | QVQLQQSGSELKKPGASVKVSCKASGYTFTNYGMNWVKQAPGQGLKW |
| domain | MGWINTYTGEPTYTDDFKGRFAFSLDTSVSTAYLQISSLKADDTAVY |
| | FCARGGFGSSYWYFDVWGQGSLVTVSS |
| Anti-TROP2 VL | DIQLTQSPSSLSASVGDRVSITCKASQDVSIAVAWYQQKPGKAPKLL |
| domain | IYSASYRYTGVPDRFSGSGSGTDFTLTISSLQPEDFAVYYCQQHYIT |
| | PLTFGAGTKVEIKR |
| Anti-GPC3 | QVQLVQSGAEVKKPGASVKVSCKASGYTFTDYEMHWVRQAPGQGLEW |
| binding domain | MGALDPKTGDTAYSQKFKGKATLTADKSTSTAYMELSSLTSEDTAVY |
| variable heavy | YCTRFYSYTYWGQGTLVTVSS |
| chain | |
| Anti-GPC3 | DVVMTQSPLSLPVTPGEPASISCRSSQSLVHSNRNTYLHWYLQKPGQ |
| binding domain | SPQLLIYKVSNRFSGVPDRFSGSGSGTDFTLKISRVEAEDVGVYYCS |
| variable light | QNTHVPPTFGQGTKLEIK |
| chain | |
| Anti-GPC3 | QVQLVQSGAEVKKPGASVKVSCKASGYTFTDYEMHWVRQAPGQGLEW |
| binding domain | MGALDPKTGDTAYSQKFKGKATLTADKSTSTAYMELSSLTSEDTAVY |
| (scFv) | YCTRFYSYTYWGQGTLVTVSSGGGGSGGGGSGGGGSDVVMTQSPLSL |
| | PVTPGEPASISCRSSQSLVHSNRNTYLHWYLQKPGQSPQLLIYKVSN |
| | RFSGVPDRFSGSGSGTDFTLKISRVEAEDVGVYYCSQNTHVPPTFGQ |
| | GTKLEIK |
| Anti-GPC3 | |
| VHH binding | |
| domains | |
| CD8a | IYIWAPLAGTCGVLLLSLVIT |
| transmembrane | |
| domain | |
| CD8a | IYIWAPLAGTCGVLLLSLVITLYC |
| transmembrane | |
| domain | |
| CD2 | IYLIIGICGGGSLLMVFVALLVFYIT |
| Transmembrane | |
| domain | |
| CD28 | FWVLVVVGGVLACYSLLVTVAFIIFWV |
| transmembrane | |
| domain | |
| CD68 | ILLPLIIGLILLGLLALVLIAFCII |
| transmembrane | |
| domain | |
| Mutant CD68 | ILLPLIIGLILLGLLALVLIAFAII |
| transmembrane | |
| domain | |
| CD8α hinge | ALSNSIMYFSHFVPVFLPAKPTTTPAPRPPTPAPTIASQPLSLRPEA |
| domain + | CRPAAGGAVHTRGLDIYIWAPLAGTCGVLLLSLVITLYC |
| transmembrane | |
| domain | |
| CD8α hinge | ALSNSIMYFSHFVPVFLPAKPTTTPAPRPPTPAPTIASQPLSLRPEA |
| domain + | CRPAAGGAVHTRGLDIYIWAPLAGTCGVLLLSLVIT |
| transmembrane | |
| domain | |
| FcRγ-chain | LYCRRLKIQVRKAAITSYEKSDGVYTGLSTRNQETYETLKHEKPPQ |
| intracellular | |
| signaling | |
| domain | |
| FcRγ-chain | LYCRLKIQVRKAAITSYEKSDGVYTGLSTRNQETYETLKHEKPPQ |
| intracellular | |
| signaling | |
| domain | |
| FcRγ-chain | RLKIQVRKAAITSYEKSDGVYTGLSTRNQETYETLKHEKPPQ |
| intracellular | |
| signaling | |
| domain | |
| FcRγ-chain | RRLKIQVRKAAITSYEKSDGVYTGLSTRNQETYETLKHEKPPQ |
| intracellular | |
| signaling | |
| domain | |
| PI3K | YEDMRGILYAAPQLRSIRGQPGPNHEEDADSYENM |
| recruitment | |
| domain | |
| CD40 | KKVAKKPTNKAPHPKQEPQEINFPDDLPGSNTAAPVQETLHGCQPVT |
| intracellular | QEDGKESRISVQERQ |
| domain | |
| TNFR1 | QRWKSKLYSIVCGKSTPEKEGELEGTTTKPLAPNPSFSPTPGFTPTL |
| intracellular | GFSPVPSSTFTSSSTYTPGDCPNFAAPRREVAPPYQGADPILATALA |
| domain | SDPIPNPLQKWEDSAHKPQSLDTDDPATLYAVVENVPPLRWKEFVRR |
| | LGLSDHEIDRLELQNGRCLREAQYSMLATWRRRTPRREATLELLGRV |
| | LRDMDLLGCLEDIEEALCGPAALPPAPSLLR |
| TNFR2 | PLCLQREAKVPHLPADKARGTQGPEQQHLLITAPSSSSSSLESSASA |
| intracellular | LDRRAPTRNQPQAPGVEASGAGEARASTGSSDSSPGGHGTQVNVTCI |
| domain | VNVCSSSDHSSQCSSQASSTMGDTDSSPSESPKDEQVPFSKEECAFR |
| | SQLETPETLLGSTEEKPLPLGVPDAGMKPS |
| CLEC9A (1-35) | MHEEEIYTSLQWDSPAPDTYQKCLSSNKCSGACCL |
| intracellular | |
| signaling | |
| domain | |

### Methods for Preparing CFPs and Engineered Myeloid Cells

The method for preparing CAR-Ps comprise the steps of (1) screening for PSR subunit framework; (2) screening for antigen binding specificity; (3) CAR-P recombinant nucleic acid constructs; (4) engineering cells and validation.

Screening for PSR subunit framework: As described above, the design of the receptor comprises at least of one phagocytic receptor domain, which enables the enhanced signaling of phagocytosis. In essence a large body of plasma membrane proteins can be screened for novel phagocytic functions or enhancements domains. Methods for screening phagocytic receptor subunits are known to one of skill in the art. Additional information can be found in The Examples section. In general, functional genomics and reverse engineering is often employed to obtain a genetic sequence encoding a functionally relevant protein polypeptide or a portion thereof. In some embodiments, primers and probes are constructed for identification, and or isolation of a protein, a polypeptide or a fragment thereof or a nucleic acid fragment encoding the same. In some embodiments, the primer or probe may be tagged for experimental identification. In some embodiments, tagging of a protein or a peptide may be useful in intracellular or extracellular localization.

Potential antibodies are screened for selecting specific antigen binding domains of high affinity. Methods of screening for antibodies or antibody domains are known to one of skill in the art. Specific examples provide further information. Examples of antibodies and fragments thereof include, but are not limited to IgAs, IgDs, IgEs, IgGs, IgMs, Fab fragments, F(ab′)2 fragments, monovalent antibodies, scFv fragments, scRv-Fc fragments, IgNARs, hcIgGs, V_{HH} antibodies, nanobodies, and alphabodies.

Commercially available antibodies can be adapted to generate extracellular domains of a chimeric receptor. Examples of commercially available antibodies include, but are not limited to: anti-HGPRT, clone 13H11.1 (EMD Millipore), anti-ROR1 (ab135669) (Abcam), anti-MUC1 [EP1024Y] (ab45167) (Abcam), anti-MUC16 [X75] (ab1107) (Abcam), anti-EGFRvIII [L8A4] (Absolute antibody), anti-Mesothelin [EPR2685 (2)] (ab134109) (Abcam), HER2 [3B5] (ab16901) (Abcam), anti-CEA (LS-C84299-1000) (LifeSpan BioSciences), anti-BCMA (ab5972) (Abcam), anti-Glypican 3 [9C2] (ab129381) (Abcam), anti-FAP (ab53066) (Abcam), anti-EphA2 [RM-0051-8F21] (ab73254) (Abcam), anti-GD2 (LS-0546315) (LifeSpan BioSciences), anti-CD19 [2E2B6B10] (ab31947) (Abcam), anti-CD20 [EP459Y] (ab78237) (Abcam), anti-CD30 [EPR4102] (ab134080) (Abcam), anti-CD33 [SP266] (ab199432) (Abcam), anti-CD123 (ab53698) (Abcam), anti-CD133 (BioLegend), anti-CD123 (1A3H4) ab181789 (Abcam), and anti-CD171 (L1.1) (Invitrogen antibodies). Techniques for creating antibody fragments, such as scFvs, from known antibodies are routine in the art.

The recombinant nucleic acid can be generated following molecular biology techniques known to one of skill in the art. The methods include but are not limited to designing primers, generating PCR amplification products, restriction digestion, ligation, cloning, gel purification of cloned product, bacterial propagation of cloned DNA, isolation and purification of cloned plasmid or vector. General guidance can be found in: Molecular Cloning of PCR Products: by Michael Finney, Paul E. Nisson, Ayoub Rashtchian in Current Protocols in Molecular Biology, Volume 56, Issue 1 (First published: 1 Nov. 2001); Recombinational Cloning by Jaehong Park, Joshua LaBaer in Current Protocols in Molecular Biology Volume 74, Issue 1 (First published: 15 May 2006) and others. In some embodiments specific amplification techniques may be used, such as TAS technique (Transcription-based Amplification System), described by Kwoh et al. in 1989; the 3SR technique, which are hereby incorporated by reference. (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

In some embodiments the recombinant nucleic acid sequence is optimized for expression in human.

DNA, mRNA and Circular RNA: In some embodiments, naked DNA or messenger RNA (mRNA) may be used to introduce the nucleic acid inside the cell. In some embodiments, DNA or mRNA encoding the PFP is introduced into the phagocytic cell by lipid nanoparticle (LNP) encapsulation. mRNA is single stranded and may be codon optimized. In some embodiments the mRNA may comprise one or more modified or unnatural bases such as 5′-Methylcytosine, or Pseudouridine. mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. LNP encapsulated DNA or RNA can be used for transfecting myeloid cells, such as macrophages, or can be administered to a subject.

In some embodiments, circular RNA (circRNAs) encoding the PFP is used. In circular RNAs (circRNAs) the 3′ and 5′ ends are covalently linked, constitute a class of RNA. CircRNA may be delivered inside a cell or a subject using LNPs.

### Bi- and Trispecific Monocyte or Macrophage Engagers

In some embodiments, the present disclosure provides compositions and methods for cancer immunotherapy. The methods provided herein help design tools that can induce resident human monocytes or macrophages to become efficient killer cells that target cancer cells and eliminate them by efficient phagocytosis. In some embodiments, the monocytes or macrophages provide sustained immunological response against the cancer cell. Various embodiments are described herein.

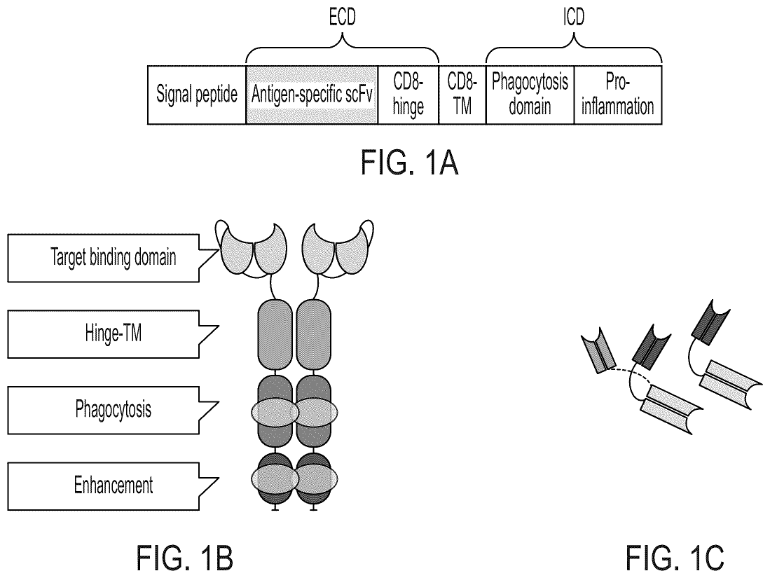

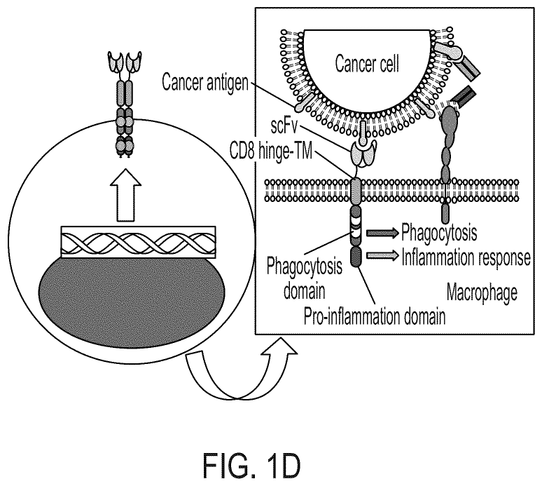

Provided herein are specific constructs and designs are disclosed for such chimeric proteins, termed chimeric “engagers”, exemplified in FIG. 1C.

In some embodiments, the chimeric engagers comprise two or more fused antibodies, each having a specific binding region on the target cell, such as cancer cell or on the monocyte or macrophage. In certain embodiments, the two or more fused antibodies or the immunofusion comprises a target binding domain operably linked by a hinge-C_{H}2-C_{H}3 domain or a hinge-C_{H}3 domain of an immunoglobulin constant region to an effector binding domain that specifically binds a cell surface component of the monocyte or macrophage.

In one aspect the chimeric protein is a bispecific monocyte or macrophage engager.

In some embodiments, a bispecific engager comprises a first therapeutic agent, wherein the first therapeutic agent comprises: (i) a first antigen binding domain that specifically interacts with an antigen of a target cell, and (ii) a second antigen binding domain that specifically interacts with an extracellular region of a receptor of a monocyte or macrophage cell. In one embodiment, the therapeutic agent is a bispecific engager. In one embodiment, the bispecific monocyte or macrophage engager comprises two antibody single chain variable regions (scFv) only (no Fc amino acid segments were included) with a flexible linker, one scFv binds a cell surface component of a target cell and the other binds a receptor on monocyte or macrophage cell surface. In full unmodified forms of IgG, the variable light chain domain (V_{L}) and the variable heavy chain domain (VH) are separate polypeptide chains, i.e., are located in the light chain and heavy chain, respectively. Interaction of the antibody light chain and an antibody heavy chain, in particular the interaction of the V_{L} and V_{H} domains, one of the epitope binding site of the antibody is formed. In contrast, in the scFv construct, but V_{L} and V_{H} domains of antibodies are included in a single polypeptide chain. The two domains are separated by flexible linkers long enough to allow self-assembly of the V_{L} and V_{H} domains into functional epitope binding site.

In some embodiments, a bispecific monocyte or macrophage engager comprises: (a) a single chain variable fragment (scFv) that binds to a cell surface component of a target cell, e.g., a cancer antigen, (b) a single chain variable fragment (scFv) that binds to a cell surface component of an effector cell, e.g. the monocyte or macrophage, (c) a short linker operably linking (a) and (b). In some embodiments, the scFvs are fused at their C-termini. Each scFv comprises a light chain variable domain, and a heavy chain variable domain, operably linked by a peptide linker. In certain embodiments, the scFvs are humanized. Humanized scFvs comprise “complementarity determining regions” (CDR) that are present on a framework of an immunoglobulin of a different species as compared to that of the parent immunoglobulin from which the CDR was derived. For example, a murine CDR may be grafted into the framework region of a human antibody to prepare the “humanized antibody.”

In some embodiments, the bispecific engager is a diabody. The bispecific diabody is constructed with a V_{L} and a V_{H} domain on a single polypeptide chain have binding specificities to different (non-identical) epitopes. Additionally, the linker connecting V_{L} and V_{H} is shorter than 12 amino acid in length that is insufficient for reassembly into a functional epitope. Generally, one polypeptide chain construct comprises V_{L} having binding specificity to a first antigen and V_{H} having binding specificity to a second antigen, and another polypeptide chain construct comprises V_{L} having binding specificity to the second antigen and V_{H} having binding specificity to the first antigen; the two polypeptide chains are allowed to self-assemble into a bi-specific diabody. In some embodiments, a cysteine residue may be introduced at the C terminus of the construct that can allow disulfide bond formation between two chains without interfering with the binding properties of the engager.

In some embodiments, the bispecific engager is a tandem-di-scFv.

In some embodiments, recombinant nucleic acid constructs can be prepared encoding the bispecific scFv engager. The recombinant nucleic acid constructs for expressing a bispecific scFv engager comprises one or more polypeptides encoding (a) a nucleic acid sequence encoding a variable domain of the target cell binding scFv light chain, a linker, a variable domain of the target cell binding scFv heavy chain; (b) a nucleic acid sequence encoding a linker; (c) a nucleic acid sequence encoding a variable domain of the effector (monocyte or macrophage) cell binding scFv light chain, a linker, a variable domain of the effector (monocyte or macrophage) cell binding scFv heavy chain. In some embodiments, the nucleic acid constructs for expressing a bispecific scFv engager comprises an N-terminal signal peptide sequence for secretion of the bispecific scFv engager.

In some embodiments, a bispecific engager comprises two single domain antibodies (V_{HH}) operably linked with a flexible linker, one VHF′ binds a cell surface component of a target cell, and the other VIM binds a receptor on a monocyte or macrophage cell surface. In some embodiments, a chimeric bispecific monocyte or macrophage engager comprises: (a) a VIM domain that binds to a cell surface component of a target cell, e.g., a cancer antigen, (b) a VIM domain that binds to a cell surface component of an effector cell, e.g. the monocyte or macrophage, (c) a short linker operably linking (a) and (b). In some embodiments the engager comprising two single domain antibodies is a nanobody.

In some embodiments, the short linker operably linking (a) and (b) may further have additional functions. In some embodiments, the peptides can bind to a specific cell surface receptor, such as, for example, a TLR receptor, and can activate a receptor mediated cell signaling pathway in the monocyte or macrophage cell. In some embodiments, the linker is designed such as to be able to bind and activate at least an inflammatory pathway in the monocyte or macrophage cell, or potentiate monocyte or macrophage mediated phagocytosis and killing of a target cell. In some embodiments, the linker peptide may have a function of blocking or inhibiting a target cell mediated downregulation of a monocyte or macrophage cell function.

In some embodiments, nucleic acid constructs for a bispecific V engager can be generated, which comprises: (a) a nucleic acid sequence encoding a (a) a V domain that binds to a cell surface component of a target cell, e.g., a cancer antigen, (b) a Vim domain that binds to a cell surface component of an effector cell, e.g. the monocyte or macrophage, (c) a short linker operably linking (a) and (b). In some embodiments, the nucleic acid constructs for expressing a bispecific scFv engager comprises an N-terminal signal peptide sequence for secretion of the bispecific scFv engager.

As is known to one of skill in the art, the nucleic acid sequences encoding the polypeptides comprising the Vim or scFv binding domains can be inserted in a suitable expression vector under one or more promoters, e.g. CMV at the 5′end, and a polyadenylation signal at the 3′-end of the sequences encoding the polypeptides.

In some embodiments, the constructs may comprise internal ribosomal entry site (IRES), e.g., a nucleic acid sequences encoding one or more polypeptides may be preceded by an IRES.

In some embodiments, the nucleic acid sequences encoding one of the polypeptides may be placed under a separate promoter control than the remaining of the expressed sequences.

In some embodiments, a bispecific engager may further comprise an antibody or a fragment thereof that binds to a cell surface component of a target cell, and an antibody or a fragment thereof that binds to a cell surface component of an effector cell.

Provided herein are further variations of an engager, a trispecific engager. A trispecific engager comprises a first therapeutic agent, wherein the first therapeutic agent comprises: a first antigen binding domain that specifically interacts with an antigen of a target cell; a second antigen binding domain that specifically interacts with an extracellular region of a first receptor of a monocyte or macrophage cell; and a third antigen binding domain that specifically interacts with an extracellular region of a second receptor of the monocyte or macrophage cell.

In some embodiments, the trispecific engager is a fused construct of three scFvs, comprising a first scFv specific to a cell surface component on a target cancer cell, a second scFv specific to a cell surface component on the monocyte or macrophage, for example, the chimeric phagocytic receptor, and a third scFv specific to another cell surface component on the monocyte or macrophage. In some embodiments, the trispecific engager is designed such that the cell surface component on the monocyte or macrophage to which the third scFv can bind, provides an additional activation signal for the monocyte or macrophage to trigger phagocytosis and killing of the target cell. In some embodiments the third scFv binds to another phagocytic receptor on the monocyte or macrophage. In some embodiments the third scFv binds to a danger associated monocyte or macrophage signaling pathway (DAMP). In some embodiments, the third scFv binds to a TLR receptor. In some embodiments, the third scFv binds to a cytokine receptor which activates the receptor and triggers monocyte or macrophage intracellular signaling. In some embodiments, the third scFv binds to a monocyte or macrophage receptor known to generate a phagocytosis inhibitory signal and that binding of the third scFv to the receptor blocks the receptor, enabling enhanced phagocytosis. In some embodiments, the third scFv binds to a receptor that engages with one or more transmembrane domains and enhances phagocytic signaling.

In some embodiments, each of the three binding domains of the trispecific engager comprises the antigen binding domain of an antibody, a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof.

In some embodiments, the binding domains of the trispecific engager are operably linked by one or more peptide linkers. In some embodiments, the one or more peptide linkers may be functional peptides that can bind to a specific cell surface receptor, such as, for example, a TLR receptor, and can activate a receptor mediated cell signaling pathway in the monocyte or macrophage cell. In some embodiments, the linker is designed such as to be able to bind and activate at least an inflammatory pathway in the monocyte or macrophage cell, or potentiate monocyte or macrophage mediated phagocytosis and killing of a target cell. In some embodiments, the linker peptide may have a function of blocking or inhibiting a target cell mediated downregulation of a monocyte or macrophage cell function.

In some embodiments, a nucleic acid constructs encoding a trispecific engager comprises one or more nucleic acid encoding (a) a polypeptide comprising an scFv domain that binds to a cell surface component of a target cell, e.g., a cancer antigen, (b) a polypeptide comprising an scFv domain that binds to a first cell surface component of an effector cell, e.g. the monocyte or macrophage, (c) a polypeptide comprising an scFv domain that binds to a second cell surface component of the monocyte or macrophage, for example, the chimeric construct constituting the second therapeutic agent; or a native monocyte or macrophage cell surface receptor, wherein each of the polypeptides are operably linked to one another. In some embodiments, a nucleic acid constructs encoding a trispecific engager comprises one or more nucleic acid encoding (a) a polypeptide comprising a VIM domain that binds to a cell surface component of a target cell, e.g., a cancer antigen, (b) a polypeptide comprising a VHF′ domain that binds to a first cell surface component of an effector cell, e.g. the monocyte or macrophage, (c) a polypeptide comprising a VIM domain that binds to a second cell surface component of the monocyte or macrophage. In some embodiments, the nucleic acid constructs for expressing a bispecific scFv engager comprises an N-terminal signal peptide sequence for secretion of the bispecific scFv engager. As contemplated herein, a skilled artisan can exchange the scFv or VIM binding sequences with a nucleic acid sequence of a short peptide encoding any suitable target region binding element. In some embodiments, the polypeptide constructs are encoded in a monocistronic construct. In some embodiments, the polypeptide constructs are encoded in a polycistronic construct. In some embodiments, one or more nucleic acid sequences encoding short linker polypeptides are inserted in between sequences encoding two polypeptides. In some embodiments, the expression of the nucleic acid sequence encoding each polypeptide is driven by a separate promoter. In some embodiments, the nucleic acid sequence encoding each polypeptide is driven by a single promoter. In some embodiments one or more IRES sequences are introduced into the construct.

In some embodiments, one or more polypeptides may be expressed separately within a cell, and which may assemble post-translationally.

In some embodiments, polypeptides may be designed to assemble on special peptide scaffolds upon secretion outside the cell.

In some embodiments, the bi- or trispecific engagers bind to an antigen on a cancer cell, selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), Mesothelin, EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFR-beta, SSEA-4, EGFR, NCAM, prostase, PAP, ELF2M, GM3, TEM7R, CLDN6, TSHR, GPRC5D, ALK, IGLL1 and combinations thereof. In some embodiments, for example, the cancer antigen for a target cancer cell can be one or more of the mutated/cancer antigens: MUC16, CCAT2, CTAG1A, CTAG1B, MAGE A1, MAGEA2, MAGEA3, MAGE A4, MAGEA6, PRAME, PCA3, MAGE C1, MAGEC2, MAGED2, AFP, MAGEA8, MAGE9, MAGEA11, MAGEA12, IL13RA2, PLAC1, SDCCAG8, LSP1, CT45A1, CT45A2, CT45A3, CT45A5, CT45A6, CT45A8, CT45A10, CT47A1, CT47A2, CT47A3, CT47A4, CT47A5, CT47A6, CT47A8, CT47A9, CT47A10, CT47A11, CT47A12, CT47B1, SAGE1, and CT55.

In some embodiments, the antigen on a cancer cell is selected from the group consisting of CD2, CD3, CD4, CD5, CD7, CCR4, CD8, CD30, CD45, CD56.

In some embodiments, the antigen is an ovarian cancer antigen or a T lymphoma antigen.

In some embodiments, for example, the cancer antigen for a target cancer cell can be one or more of the mutated/cancer antigens: IDH1, ATRX, PRL3, or ETBR, where the cancer is a glioblastoma.

In some embodiments, for example, the cancer antigen for a target cancer cell can be one or more of the mutated/cancer antigens: CA125, beta-hCG, urinary gonadotropin fragment, AFP, CEA, SCC, inhibin or extradiol, where the cancer is ovarian cancer.

In some embodiments the cancer antigen for a target cancer cell may be CD5.

In some embodiments the cancer antigen for a target cancer cell may be HER2.

In some embodiments the cancer antigen for a target cancer cell may be EGFR Variant III.

In some embodiments the cancer antigen for a target cancer cell may be CD19.

In some embodiments, the antigen is an integrin receptor.

In some embodiments, the antigen is an integrin receptor selected from the group consisting of α1, α2, αIIb, α3, α4, α5, α6, α7, α8, α9, α10, α11, αD, αE, αL, αM, αV, αX, β 1, β 2, β 3, β 4, β 5, β 6, β 7, and β8. In some embodiments, the bi- or trispecific engager binds to an extracellular domain of a monocyte or macrophage receptor from a member of the integrin β₂ subfamily α_{Mβ}_{₂} (CD11b/CD18, Mac-1, CR3, Mo-1), α_{L}β₂ (CD11a/CD18, LFA-1), α_{Xβ}_{₂} (CD11c/CD18), and α_{Dβ}_{₂} (CD11d/CD18).

Provided herein are exemplary target cell binders (e.g., engagers) that can specifically bind to a cell surface molecule (such as a cell surface antigen) on a cancer cell. In some embodiments, the binder is an antibody specific to the antigen, or a fragment thereof. In some embodiments, the binder comprises a scFv, or a fragment thereof, that specifically binds to an antigen on a tumor cell. In some embodiments, the antigen on a tumor cell is CD5. The binder comprises a heavy chain (HC) sequence and a light chain (LC) sequence. An scFv specific for CD5 (anti-CD5 scFv) comprises an amino acid sequence corresponding to a variable heavy chain (VH) domain and an amino acid sequence corresponding to a (V_{L}).

In some embodiments, the recombinant polynucleic acids encoding the bi-specific, tri-specific or multispecific engagers are incorporated in a myeloid cell population ex vivo, e.g., by electroporation and then administered to a subject in need thereof.

In some embodiments, a bi-specific, tri-specific or multispecific engager can be directed, at least via one arm of the molecule to engage an NK cell or a T cell. In some embodiments, an NK cell engager is also termed a BiKE, or a TRiKE depending on whether the engager is bi- or tri-specific respectively and that it binds to a surface molecule on an NK cell.

T cell. In some embodiments, a T cell engager is termed a BiTE, or a TRiTE depending on whether the engager is bi- or tri-specific respectively and that it binds to a surface molecule on an T cell. In some embodiments combination therapies of BiME/TRiME with BiKEs or TriKEs or BiTEs or TRiTEs or other cellular therapies are encompassed in the scope of the disclosure.

In some embodiments, the therapy involving bi-specific, tri-specific or multispecific engagers in cancer therapy is preceded by or accompanied by administering to the subject, one or more components for reprogramming of TAMs or the TME. In some embodiments, drugs affecting the TME, for example, anti-angiogenic drugs, immune checkpoints inhibitors, drugs targeting macrophages, such as kinase inhibitors or antibodies directed to the CSF-1 receptor may be used as a preconditioning agent prior to administering a myeloid cell therapeutic.

In some embodiment, the immune cell inhibitory agent may comprise an agent or component that is a Tie-2 inhibitor. In some embodiment, the immune cell inhibitory agent may comprise an agent or component that is a CD40 agonist. In some embodiment, the immune cell inhibitory agent may comprise an agent or component that is a PD1/PDL1 inhibitor as exemplified above. In some embodiment, the immune cell inhibitory agent may comprise an agent or component that is a CCR5/CCL5 inhibitor. In some embodiment, the immune cell inhibitory agent may comprise an agent or component for targeting MARCO, thereby specifically targeting macrophages, and leaving other cells unaffected. In some embodiment, the immune cell inhibitory agent may comprise an agent or component for specifically targeting PI3 μg/HDAC class Ha targeting. In some embodiments, the immune cell inhibitor is an immune cell modulator selected from the group consisting of a TLR-agonist, a DICER inhibitor, an HDAC inhibitor, a PI3-Kinase inhibitor and a myeloid cell surface binding agent.

In some embodiments, the immune cell inhibitory agent may be an inhibitor of poly ADP ribose polymerase (PARP). PARP possesses enzymatic ability to synthesize and attach poly (ADP-ribose) (also known as PAR) to different protein substrates by a post-translational modification. PARP inhibitors act as antitumor agents. An exemplary PARP inhibitor is olaparib.

In some embodiments, the immune cell inhibitory agent is trabectedin or lurbinectedin. Trabectedin (ET743) is an anti-cancer drug that directly perturb the DNA metabolism. Lurbinectedin (PM01183) is a derivative of Trabectedin. Both drugs were shown to induce degradation of the RNA polymerase II (RNAPII) through the ubiquitin—proteasome pathway, and is shown to inhibit the transcription of selected cytokines (e.g., CCL2, IL6, IL8, PTX3) by TAMs abrogating their protumoral properties and modifying the tumor microenvironment. In some embodiments, the immune cell inhibitory agent is an HDAC inhibitor. In some embodiments, the immune cell inhibitory agent comprises an anticancer drug, romidepsin. In some embodiments, the immune cell inhibitory agent comprises a VEGF inhibitor. In some embodiments, the immune cell inhibitory agent comprising a VEGF inhibitor is bevacizumab. In some embodiments, the immune cell inhibitory agent is anti-VEGFR2 antibody ramucirumab. In some embodiments, the immune cell inhibitory agent is a small molecule inhibitor of VEGF receptors VEGFR1/2/3, (these agents also block PDGFR-β, cKit, BRAF, FLT3 and CSF1R among other receptor tyrosine kinases) including sunitinib, axitinib, or sorafenib. In some embodiments, the immune cell inhibitory agent comprises a suitable dose of Avelumab and/or Bevacizumab. In some embodiments, the immune cell inhibitory agent comprises a suitable dose of Atezolizumab and/or Bevacizumab. In some embodiments, the immune cell inhibitory agent comprises a suitable dose of Nivolumab and/or Bevacizumab. In some embodiments, the immune cell inhibitory agent comprises a suitable dose of Ramucirumab and/or Paclitaxel. In some embodiments, the immune cell inhibitory agent comprises a suitable dose of Cabozantinib and/or Ipilumab. In some embodiments, the immune cell inhibitory agent comprises a suitable dose of Pembrolizumab and/or axitinib.

In some embodiments, the immune cell modulator is a IL-10, TGF-b, IL-4, an anti-CD41 agent, an anti-PD1 agent or an arginase inhibitor.

In some embodiments, cell therapy the preconditioning agent or the immune cell inhibitory agent is a myeloid and/or stromal checkpoint inhibitor. In some embodiments, preconditioning agent or the immune cell inhibitory agent is a monoclonal antibody that binds to and inhibits the inhibitory receptor LAIR1.

### Polynucleotide Delivery

In some embodiments a polynucleotide, e.g., an RNA polynucleotide, is introduced or incorporated in the cell, e.g., a monocyte, by known methods of transfection, such as using lipofectamine, or calcium phosphate, or via physical means such as electroporation or nucleofection. In some embodiments the polynucleotide is introduced or incorporated in the cell by infection, a process commonly known as viral transduction. In some embodiments, a polynucleotide is introduced or incorporated into the cell via a lipid nanoparticle or a polymer nanoparticle.

Lipid nanoparticles (LNP) may comprise a polar and or a nonpolar lipid. In some embodiments, cholesterol is present in the LNPs for efficient delivery. LNPs are 100-300 nm in diameter provide efficient means of RNA delivery to various cell types. In some embodiments, LNP may be used to introduce the recombinant nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an RNA molecule. In some embodiments, the LNP encapsulates the nucleic acid wherein the nucleic acid is inserted in a vector, such as a plasmid vector. In some embodiments, the LNP encapsulates the nucleic acid wherein the nucleic acid is a circular RNA (circRNA) molecule.

It is well known that a nucleic acid, e.g., a messenger ribonucleic acid (mRNA), may be delivered inside a cell, whether in vitro, in vivo, in situ or ex vivo, to cause intracellular translation of the nucleic acid and production of an encoded polypeptide of interest. Because of their unique closed circular structure, circRNAs are more resistant to the degradation by exonuclease and have a longer half-life than their corresponding linear counterparts. As such, it is desirable to develop new and improved circRNAs which are useful in the production of polypeptides of interest. In an embodiment, circular RNA and/or methods of producing circular RNA is described in US patent applications US20160194368A1 and/or US20180169146A1.

In some embodiments, an mRNA encoding a chimeric fusion protein, encapsulated by a suitable LNP is designed for in vivo delivery that can be targeted past the liver of the subject. In some embodiments, the LNP-encapsulated mRNA is designed for uptake and expression in a myeloid cell in the body. In some embodiments, the LNP-encapsulated mRNA is designed for expression and/or function specifically in a myeloid cell, e.g., in a monocyte or a macrophage. In some embodiments the engineered myeloid cells are endowed with enhanced chemotaxis ability, and enhanced target cell phagocytosis, and remains plastic to the environmental stimuli, e.g., can be activated by the environment to act as an effector myeloid cell. In some embodiments a myeloid cell, engineered in vivo or ex vivo retains its ability to function as an activated M1 cell, and is capable of actively destroying a target cell, e.g., a cancer cell. In some embodiments a myeloid cell, engineered in vivo or ex vivo retains its ability to function as an activated M1 cell, actively destroys a target cell, e.g., a cancer cell. In some embodiments, the engineered myeloid cell is capable of actively modulating the tumor microenvironment, and actively destroys tumor cells.

### Treatment Methods

Provided herein are methods for treating cancer in a subject using a pharmaceutical composition comprising engineered myeloid cells, such as phagocytic cells (e.g., macrophages), expressing a recombinant nucleic acid encoding a CFP, such as a phagocytic receptor (PR) fusion protein (PFP), to target, attack and kill cancer cells directly or indirectly. The engineered myeloid cells, such as phagocytic cells, are also designated as CAR-P cells in the descriptions herein.

Cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In general, cellular immunotherapy comprises providing the patient a medicament comprising live cells. In some aspects a patient or a subject having cancer, is treated with autologous cells, the method comprising, isolation of PBMC-derived myeloid cells, such as macrophages, modifying the cells ex vivo to generate phagocytic myeloid cells capable of tumor lysis by introducing into the cells a recombinant nucleic acid encoding a CFP, and administering the modified myeloid cells into the subject.

In some aspects, a subject is administered one or more doses of a pharmaceutical composition comprising therapeutic myeloid cells, such as phagocytic cells, wherein the cells are allogeneic. An HLA may be matched for compatibility with the subject, and such that the cells do not lead to graft versus Host Disease, GVHD. A subject arriving at the clinic is HLA typed for determining the HLA antigens expressed by the subject.

HLA-typing is conventionally carried out by either serological methods using antibodies or by PCR-based methods such as Sequence Specific Oligonucleotide Probe Hybridization (SSOP), or Sequence Based Typing (SBT).

The sequence information may be identified by either sequencing methods or methods employing mass spectrometry, such as liquid chromatography—mass spectrometry (LC-MS or LC-MS/MS, or alternatively HPLC-MS or HPLC-MS/MS). These sequencing methods may be well-known to a skilled person and are reviewed in Medzihradszky K F and Chalkley R J. Mass Spectrom Rev. 2015 January-February; 34(1):43-63.

In some aspects, the phagocytic cell is derived from the subject, transfected or transduced with the recombinant nucleic acid in vitro, expanded in cell culture in vitro for achieving a number suitable for administration, and then administered to the subject. In some embodiments, the steps of transfected or transduced with the recombinant nucleic acid in vitro, expanded in cell culture in vitro for achieving a number suitable for administration takes 2 days, or 3 days, or 4 days or 5 days or 6 days or 7 days or 8 days or 9 days or 10 days.

In some embodiments, sufficient quantities of transfected or transduced myeloid cells, such as macrophages, comprising the recombinant nucleic acid are preserved aseptically, which are administered to the subject as “off the shelf” products after HLA typing and matching the product with the recipients HLA subtypes. In some embodiments, the engineered phagocytes are cryopreserved. In some embodiments, the engineered phagocytes are cryopreserved in suitable media to withstand thawing without considerable loss in cell viability.

In some embodiment, the subject is administered a pharmaceutical composition comprising the DNA, or the mRNA or the circRNA in a vector, or in a pharmaceutically acceptable excipient described above.

In some embodiments the administration of the off the shelf cellular products may be instantaneous, or may require 1 day, 2 days or 3 days or 4 days or 5 days or 6 days or 7 days or more prior to administration. The pharmaceutical composition comprising cell, or nucleic acid may be preserved over time from preparation until use in frozen condition. In some embodiments, the pharmaceutical composition may be thawed once. In some embodiments, the pharmaceutical composition may be thawed more than once. In some embodiments, the pharmaceutical composition is stabilized after a freeze-thaw cycle prior administering to the subject. In some embodiments the pharmaceutical composition is tested for final quality control after thawing prior administration.

In some embodiments, the human subject has been lymphodepleted prior to administration of the population of cells.

In some embodiments, the population of cells is autologous or from the human subject.

In some embodiments, the population of cells is allogeneic. In some embodiments, the population of cells is from a healthy donor. In some embodiments, the population of cells is a population of non-engineered cells. In some embodiments, the population of cells is a population of cells with an HLA haplotype matched to the HLA haplotype of the human subject. In some embodiments, the population of cells is a population of cells with an HLA haplotype that is not matched to the HLA haplotype of the human subject.

In some embodiments, the population of cells is derived from a population of genetically modified cells. In some embodiments, the population of genetically modified cells has been genetically engineered to lack expression of one or more HLA alleles, one or more class I HLA alleles, or all class I HLA alleles. In some embodiments, the population of cells is derived from a population of genetically modified stem cells. In some embodiments, the population of genetically modified stem cells is a population of genetically modified pluripotent stem cells. In some embodiments, the population of genetically modified pluripotent stem cells is a population of genetically modified induced pluripotent stem cells (iPSCs).

In some embodiments, the method further comprises administering a second dose of the population of cells. In some embodiments, the population of cells of the second dose is autologous or from the human subject. In some embodiments, a first dose of the population of cells is allogeneic. In some embodiments, the population of cells of the second dose is allogeneic. In some embodiments, a first dose of the population of cells is allogeneic. In some embodiments, the population of cells of the second dose that is allogeneic is HLA-type mismatched to HLA-type of the population of cells of the first dose that is allogeneic. In some embodiments, the human subject elicits an immune response to the population of cells of the first dose that is allogeneic. In some embodiments, the method further comprises administering 1, 2, 3, 4, 5, 6, 7, 8, 9 or 10 additional doses of the population of cells.

The present disclosure provides methods for producing genetically modified cells using engineered nucleases that recognize and cleave recognition sequences found within the genome of the population of cells, such as HLA genes. Cleavage at such recognition sequences can allow for NHEJ at the cleavage site and disrupted expression of the MHCs encoded by the HLA genes, leading to reduced expression and/or function of the MHCs at the cell surface. Additionally, cleavage at such recognition sequences can further allow for homologous recombination of exogenous nucleic acid sequences directly into the HLA genes.

In examples where the genetically modified cells of the invention are human monocytes or stem cells or cells derived therefrom, such as human monocytes, such cells may require activation prior to introduction of a nuclease, such as a recombinant meganuclease, a recombinant zinc-finger nuclease (ZFN), a recombinant transcription activator-like effector nuclease (TALEN), a CRISPR/Cas nuclease, or a megaTAL nuclease; and/or an exogenous nucleic acid sequence.

In some embodiments, the invention provides a pharmaceutical composition comprising a genetically modified cell of the invention, or a population of genetically modified cells of the invention, and a pharmaceutical carrier. Such pharmaceutical compositions can be prepared in accordance with known techniques. See, e.g., Remington, The Science And Practice of Pharmacy (21s ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, where the genetically modified cell is a genetically modified human monocyte or stem cell or cell derived therefrom, such as a human monocyte, pharmaceutical compositions of the invention can further include biological molecules which promote in vivo cell proliferation, differentiation, invasion, and/or and engraftment. Pharmaceutical compositions comprising genetically modified cells of the invention can be administered in the same composition as an additional agent or biological molecule or, alternatively, can be co-administered in separate compositions.

In some embodiments, the subject is a human subject. In some embodiments, the method is effective to treat or reduce the symptoms of the cancer. In some embodiments, the method is effective to treat or prevent host-vs-graft disease. In some embodiments, the immunotherapy is an allogeneic cellular immunotherapy.

In some embodiments, the genetically modified cells are generated by inserting an exogenous polynucleotide encoding the CAR within a chromosome of a cell by a method comprising transfecting the cell with one or more nucleic acids including: (a) a first nucleic acid comprising a polynucleotide encoding an engineered nuclease having specificity for a recognition sequence within the chromosome, wherein the engineered nuclease is expressed in the cell; and (b) a template nucleic acid comprising the exogenous polynucleotide; wherein the engineered nuclease generates a cleavage site within the chromosome at the recognition sequence, and wherein the exogenous polynucleotide encoding the CAR is inserted into the chromosome at the cleavage site.

In some embodiments, the template nucleic acid is introduced into the cell using a viral vector. In certain embodiments, the viral vector is a recombinant AAV vector.

In some embodiments, the engineered nuclease is an engineered meganuclease, a zinc finger nuclease, a TALEN, a compact TALEN, a CRISPR system nuclease, or a megaTAL. In some embodiments, the engineered nuclease is an engineered meganuclease.

In another aspect, provided herein is a kit comprising: (a) immune cell inhibitory agent; and (b) a composition comprising a population of genetically modified cells, wherein the population of genetically modified cells comprise in their genome an exogenous polynucleotide encoding a chimeric antigen receptor (CAR) that is expressed by the genetically modified cells. In some embodiments, the immune cell inhibitory agent is a lymphodepleting chemotherapeutic agent.

In another aspect, provided herein is a kit comprising: (a) a lymphodepleting chemotherapeutic agent, (b) an additional immune cell inhibitory agent; and (c) a composition comprising a population of genetically-modified cells, wherein the population of genetically modified cells comprise in their genome an exogenous polynucleotide encoding a chimeric antigen receptor (CAR) that is expressed by the genetically modified cells.

In some embodiments of a kit provided herein, the lymphodepleting chemotherapeutic agent is fludarabine, cyclophosphamide, or a combination thereof.

In some embodiments, the exogenous polynucleotide is within a target gene in the genome of the genetically modified cell. In certain embodiments, the target gene is selected from the group consisting of a class I HLA gene and a class II HLA gene. In certain embodiments, the target gene is selected from the group consisting of an HLA-A gene, an HLA-B gene and an HLA-C gene. In some embodiments, the genetically modified cell is a human cell, or a cell derived therefrom. In some embodiments, the CAR specifically binds to a molecule on the surface of a cancer cell. In some embodiments, the kit further comprises instructions for use of components of the kit in treating a cancer.

In another aspect, the invention provides a method for reducing the number of target cells in a subject, wherein the method comprises: (a) administering to the subject a lymphodepletion regimen that comprises administering one or more effective doses of an immune cell inhibitory agent; and (b) administering to the subject an effective dose of a pharmaceutical composition comprising a population of human immune cells, wherein a plurality of the human immune cells are genetically modified human immune cells that express a CAR; wherein the CAR comprises an extracellular ligand-binding domain having specificity for an antigen on the target cells.

In some embodiments, the genetically modified human immune cells comprise an inactivated HLA gene. In some embodiments, the genetically modified human immune cells comprise an inactivated class I HLA gene. In some embodiments, each HLA class I gene of the genetically modified human immune cells is inactivated.

In some embodiments, the one or more effective doses of the immune cell inhibitory agent depletes a population of endogenous lymphocytes in the subject.

In some embodiments, the immune cell inhibitory agent is selected from the group consisting of a monoclonal antibody, a polyclonal antibody, a humanized antibody, a fully human antibody, a bispecific antibody, a dual-variable immunoglobulin domain, a single-chain Fv molecule (scFv), a sdAb, a diabody, a triabody, a nanobody, an antibody-like protein scaffold, a Fv fragment, a Fab fragment, a F(ab′)2 molecule, and a tandem di-scFv.

In some embodiments, the immune cell inhibitory agent does not detectably bind the genetically modified human immune cells.

In some embodiments, the immune cell inhibitory agent is administered to the subject prior to administration of the pharmaceutical composition. In certain embodiments, the immune cell inhibitory agent is administered to the subject concurrently with administration of the pharmaceutical composition. In certain embodiments, the immune cell inhibitory agent is administered to the subject following administration of the pharmaceutical composition. In some embodiments, the immune cell inhibitory agent is administered to the subject 1-30 days prior to administration of the pharmaceutical composition. In some embodiments, the immune cell inhibitory agent is administered to the subject within 10 days prior to administration of the pharmaceutical composition. In some embodiments, the immune cell inhibitory agent is administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more prior to administration of the pharmaceutical composition. In some embodiments, the immune cell inhibitory agent is administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more following administration of the pharmaceutical composition.

In some embodiments, the immune cell inhibitory agent is administered intravenously. In some embodiments, the immune cell inhibitory agent is administered orally. In some embodiments, the immune cell inhibitory agent is administered subcutaneously.

In some embodiments, the pharmaceutical composition is administered at a dose of between about 1×10⁴ and about 1×10⁸ genetically modified human immune cells/kg. In some embodiments, the pharmaceutical composition is administered at a dose of between about 1×10⁵ and about 1×10⁷ genetically modified human immune cells/kg. In some embodiments, the pharmaceutical composition is administered at a dose of between about 1×10⁵ and about 6×10⁶ genetically modified human immune cells/kg. In some embodiments, the pharmaceutical composition is administered at a dose of between about 3×10⁵ and about 6×10⁶ genetically modified human immune cells/kg. In some embodiments, the pharmaceutical composition is administered at a dose of between about 3×10⁵ and about 3×10⁶ genetically modified human immune cells/kg. In some embodiments, the pharmaceutical composition is administered at a dose of about 0.5×10⁶ genetically modified human immune cells/kg. In some embodiments, the pharmaceutical composition is administered at a dose of about 1.0×10⁶ genetically modified human immune cells/kg. In some embodiments, the pharmaceutical composition is administered at a dose of about 2.0×10⁶ genetically modified human immune cells/kg. In some embodiments, the pharmaceutical composition is administered at a dose of about 3.0×10⁶ genetically modified human immune cells/kg. In some embodiments, the dose of the pharmaceutical composition comprises no more than 3×10⁵ genetically modified human immune cells.

In some embodiments, the method further comprises administering a second dose of the pharmaceutical composition to the subject.

In some embodiments, the method further comprises administering one or more effective doses of one or more lymphodepleting agents to the subject prior to administration of the pharmaceutical composition.

In some embodiments, the lymphodepleting agent is administered to the subject prior to administration of the additional immune cell inhibitory agent and prior to administration of the pharmaceutical composition. In certain embodiments, the lymphodepleting agent is administered to the subject concurrently with administration of the additional immune cell inhibitory agent and prior to administration of the pharmaceutical composition. In some embodiments, the lymphodepleting agent is administered to the subject following administration of the additional immune cell inhibitory agent and prior to administration of the pharmaceutical composition.

In some embodiments, the lymphodepleting agent is fludarabine, cyclophosphamide, bendamustine, melphalan, 6-mercaptopurine (6-MP), daunorubicin, cytarabine, L-asparaginase, methotrexate, prednisone, dexamethasone, nelarabine, or a combination thereof. In certain embodiments, the lymphodepleting agent is cyclophosphamide. In some embodiments, cyclophosphamide is administered to the subject at a dose of about 250-1500 mg/m²/day. In certain embodiments, cyclophosphamide is administered to the subject at a dose of about 500-1000 mg/m²/day. In some embodiments, the dose of cyclophosphamide is about 250-1500 mg/m²/day, about 300-1500 mg/m²/day, about 350-1500 mg/m²/day, about 400-1500 mg/m²/day, about 450-1500 mg/m²/day, about 500-1500 mg/m²/day, about 550-1500 mg/m²/day, or about 600-1500 mg/m²/day. In another embodiment, the dose of cyclophosphamide is about 250-1500 mg/m²/day, about 350-1000 mg/m²/day, about 400-900 mg/m²/day, about 450-800 mg/m²/day, about 450-700 mg/m²/day, about 450-600 mg/m²/day, or about 450-550 mg/m²/day. In some embodiments, the dose of cyclophosphamide is about 250 mg/m²/day, about 350 mg/m²/day, about 400 mg/m²/day, about 450 mg/m²/day, about 500 mg/m²/day, about 550 mg/m²/day, about 600 mg/m²/day, about 650 mg/m²/day, about 700 mg/m²/day, about 800 mg/m²/day, about 900 mg/m²/day, or about 1000 mg/m²/day. In some embodiments, cyclophosphamide is administered to the subject at a dose of about 500-1000 mg/m²/day. In some embodiments, cyclophosphamide is administered to the subject at a dose of about 500 mg/m²/day. In some embodiments, cyclophosphamide is administered to the subject at a dose of about 500 mg/m²/day daily starting five days and ending two days prior to administration of the pharmaceutical composition. In some embodiments, cyclophosphamide is administered to the subject at a dose of about 500 mg/m²/day daily starting five days and ending three days prior to administration of the pharmaceutical composition. In some embodiments, cyclophosphamide is administered to the subject at a dose of about 1000 mg/m²/day. In some embodiments, cyclophosphamide is administered to the subject at a dose of about 1000 mg/m²/day daily starting four days and ending two days prior to administration of the pharmaceutical composition. In some embodiments, cyclophosphamide is administered to the subject at a dose of about 1000 mg/m²/day daily starting four days and ending three days prior to administration of the pharmaceutical composition.

In certain embodiments, the lymphodepleting agent is fludarabine. In some embodiments, fludarabine is administered to the subject at a dose of 10-40 mg/m²/day. In some embodiments, the dose of fludarabine is about 10-100 mg/m²/day, about 15-100 mg/m²/day, about 20-100 mg/m²/day, about 25-900 mg/m²/day, about 30-900 mg/m²/day, about 35-100 mg/m²/day, about 40-100 mg/m²/day, about 45-100 mg/m²/day, about 50-100 mg/m²/day, about 55-100 mg/m²/day, or about 60-100 mg/m²/day. In other embodiments, the dose of fludarabine is about 10-100 mg/m²/day, about 10-90 mg/m²/day, about 10-80 mg/m²/day, about 10-70 mg/m²/day, about 10-60 mg/m²/day, about 10-50 mg/m²/day, about 10-45 mg/m²/day, about 20-40 mg/m²/day, about 25-35 mg/m²/day, or about 28-32 mg/m²/day. In certain embodiments, the dose of fludarabine is about 10 mg/m²/day, 15 mg/m²/day, 20 mg/m²/day, 25 mg/m²/day, 30 mg/m²/day, 35 mg/m²/day, about 40 mg/m²/day, about 45 mg/m²/day, about 50 mg/m²/day, about 55 mg/m²/day, about 60 mg/m²/day, about 65 mg/m²/day, about 70 mg/m²/day, about 75 mg/m²/day, about 80 mg/m²/day, about 85 mg/m²/day, about 90 mg/m²/day, about 95 mg/m²/day, or about 100 mg/m²/day. In some embodiments, fludarabine is administered to the subject at a dose of 30 mg/m²/day. In some embodiments, fludarabine is administered to the subject at a dose of about 30 mg/m²/day daily starting five days and ending two days prior to administration of the pharmaceutical composition. In some embodiments, fludarabine is administered to the subject at a dose of about 30 mg/m²/day daily starting five days and ending three days prior to administration of the pharmaceutical composition. In some embodiments, fludarabine is administered to the subject at a dose of about 30 mg/m²/day daily starting seven days and ending two to three days prior to administration of the pharmaceutical composition.

In some embodiments, a composition comprising 10\hat{ }6 engineered cells are administered per administration dose. In some embodiments, a composition comprising 10\hat{ }7 engineered cells are administered per administration dose. In some embodiments, a composition comprising 5×10\hat{ }9 engineered cells are administered per administration dose. In some embodiments, a composition comprising 10\hat{ }8 engineered cells are administered per administration dose. In some embodiments, a composition comprising 2×10\hat{ }8 engineered cells are administered per administration dose. In some embodiments, a composition comprising 5×10\hat{ }8 engineered cells are administered per administration dose. In some embodiments, a composition comprising 10\hat{ }9 engineered cells are administered per administration dose. In some embodiments, a composition comprising 10\hat{ }10 engineered cells are administered per administration dose.

In some embodiments, the myeloid cell is engineered (i) to disrupt the expression of a protein encoded by an HLA in the cell, and (ii) express the CAR that is designed to bind to a target cell, e.g., a cancer cell and to activate the myeloid cell to phagocytose and lyse the target cell In some embodiments the nucleic acid sequence encoding the CAR is targeted and inserted into the genome of the myeloid cell that functionally disrupts an HLA expression, and expresses the CAR in the myeloid cell.

In one embodiment, a subject is administered an agent for lymphodepletion, and followed by an administration of the engineered myeloid cells. One advantage of using myeloid cells is that they do not cause graft-versus-host effect. Lymphodepletion in the subject reduces or eliminates the possibility of host-versus-graft disease, at least during the “window of opportunity” when the lymphodepletion is in effect. Because myeloid cells are short-lived, the method provides an effective way of treating, such as, attacking and reducing the tumor cell burden within the window of opportunity, and then following up with one or more different therapeutic compositions, which might include additional cell therapy, such as myeloid cell therapy. In some embodiments, using allogeneic myeloid cells provides an advantage when coupled with lymphodepletion, as allogeneic myeloid cells derived from a healthy donor may be more potent and aggressive in attacking and killing target cells, e.g., the cancer cells. In some embodiments, the allogeneic myeloid cells (e.g., allogeneic engineered myeloid cells) are administered at least once or at least twice or at least three times during the window of opportunity, and/or followed with administering autologous engineered myeloid cells. In some embodiments, the allogeneic myeloid cells, e.g. the engineered allogeneic myeloid cells are HLA matched with the subject. In some embodiments, the allogeneic myeloid cells are engineered to express no protein encoded by an HLA, and is referred to as a stealth myeloid cell, or a stealth monocyte.

In some embodiments, the subject is administered one or more agents to prevent recruitment of endogenous macrophages to the site of tumor, as disclosed elsewhere in the specification, in addition to a lymphodepletion treatment, whereas, allogeneic myeloid cells, (e.g. engineered allogeneic myeloid cells) may be directly injected or infused or otherwise administered as easily understood by one of skill in the art, at the site of the tumor.

In some embodiments, provided herein is a method of producing a cell bank, comprising allogeneic myeloid cells, expressing a CAR, wherein the myeloid cell is a differentiated monocyte, a monocyte precursor cell or a pluripotent cell that can be readily differentiated into a myeloid cell, whether in vitro or programmed to do so, in vivo.

In some embodiments, the engineered myeloid cells, such as phagocytic cells, are administered once.

In some embodiments, the engineered myeloid cells, such as phagocytic cells, are administered more than once.

In some embodiments, the engineered myeloid cells, such as phagocytic cells, are twice, thrice, four times, five times, six times, seven times, eight times, nine times, or ten times or more to a subject over a span of time comprising a few months, a year or more.

In some embodiments, the engineered myeloid cells, such as phagocytic cells, are administered twice weekly.

In some embodiments, the engineered myeloid cells, such as phagocytic cells, are administered once weekly.

In some embodiments, the engineered myeloid cells, such as phagocytic cells, are administered once every two weeks.

In some embodiments, the engineered myeloid cells, such as phagocytic cells, are administered once every three weeks.

In some embodiments, the engineered myeloid cells, such as phagocytic cells, are administered once monthly.

In some embodiments, the engineered phagocytic cells are administered once in every 2 months, once in every 3 months, once in every 4 months, once in every 5 months or once in every 6 months.

In some embodiments, the engineered myeloid cells, such as phagocytic cells, are administered by injection.

In some embodiments, the engineered myeloid cells, such as phagocytic cells, are administered by infusion.

In some embodiments, the engineered myeloid cells, such as phagocytic cells, are administered by intravenous infusion.

In some embodiments, the engineered myeloid cells, such as phagocytic cells, are administered by subcutaneous infusion.

The pharmaceutical composition comprising the recombinant nucleic acid or the engineered cells may be administered by any route which results in a therapeutically effective outcome. These include, but are not limited to enteral (into the intestine), gastroenteral, epidural (into the dura mater), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

In some embodiments, the subject is administered a pharmaceutical composition comprising the nucleic acid encoding the CFP or PFP as described herein. In some embodiments, the subject is administered a pharmaceutical composition comprising DNA, mRNA, or circRNA. In some embodiments, the subject is administered a vector harboring the nucleic acid, e.g., DNA, mRNA, or circRNA. In some embodiments, the nucleic acid is administered or in a pharmaceutically acceptable excipient described above.

In some embodiments, the subject is administered a nanoparticle (NP) associated with the nucleic acid, e.g. a DNA, an mRNA, or a circRNA encoding the CFP or PFP as described herein. In some embodiments, the nucleic acid is encapsulated in the nanoparticle. In some embodiments, the nucleic acid is conjugated to the nanoparticle. In some embodiments, the NP is a polylactide-co-glycolide (PGLA) particle. In some embodiments, the NP is administered subcutaneously. In some embodiments, the NP is administered intravenously. In some embodiments, the NP is engineered in relation to the administration route. For example, the size, shape, or charges of the NP maybe engineered according to the administration route. In some embodiments, subcutaneously administered NPs are less than 200 nm in size. In some embodiments, subcutaneously administered NPs are more than 200 nm in size. In some embodiments, subcutaneously administered NPs are at least 30 nm in size. In some embodiments, the NPs are intravenously infused. In some embodiments, intravenously infused NPs are at least 5 nm in diameter. In some embodiments, intravenously infused NPs are at least 30 nm in diameter. In some embodiments, intravenously infused NPs are at least 100 nm in diameter. In certain embodiments, the administered NPs, e.g. intravenously administered NPs, are engulfed by circulating monocytes. Additional NP design and administration approaches are described in Getts et al., Trends Immunol. 36(7): 419-427 (2015), the entirety of which is incorporated herein by reference.

In some embodiments, the subject is administered a pharmaceutical composition comprising a circRNA encoding the CFP or PFP as described herein. The circRNA may be administered in any route as described herein. In some embodiments, the circRNA may be directly infused. In some embodiments, the circRNA may be in a formulation or solution comprising one or more of sodium chloride, calcium chloride, phosphate and/or EDTA. In some embodiment, the circRNA solution may include one or more of saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In some embodiments, the circRNA solution is lyophilized. The amount of each component may be varied to enable consistent, reproducible higher concentration saline or simple buffer formulations. The components may also be varied in order to increase the stability of circRNA in the buffer solution over a period of time and/or under a variety of conditions. In some embodiments, the circRNA is formulated in a lyophilized gel-phase liposomal composition. In some embodiments, the circRNA formulation comprises a bulking agent, e.g. sucrose, trehalose, mannitol, glycine, lactose and/or raffinose, to impart a desired consistency to the formulation and/or stabilization of formulation components. Additional formulation and administration approaches for circRNA as described in US Publications No. US2012060293, and US20170204422 are herein incorporated by reference in entirety.

In some embodiments, the subject is administered a pharmaceutical composition comprising a mRNA encoding the CFP or PFP as described herein. In some embodiments, the mRNA is co-formulated into nanoparticles (NPs), such as lipid nanoparticles (LNPs). For example, the LNP may comprise cationic lipids or ionizable lipids. In some embodiments, the mRNA is formulated into polymeric particles, for example, polyethyleneimine particles, poly(glycoamidoamine), ly(β-amino)esters (PBAEs), PEG particles, ceramide-PEGs, polyamindoamine particles, or polylactic-co-glycolic acid particles (PLGA). In some embodiments, the mRNA is administered by direct injection. In some embodiments, the mRNA is complexed with transfection agents, e.g. Lipofectamine 2000, jetPEI, RNAiMAX, or Invivofectamine.

The mRNA may be a naked mRNA. The mRNA may be modified or unmodified. For example, the mRNA may be chemically modified. In some embodiments, nucleobases and/or sequences of the mRNA are modified to increase stability and half-life of the mRNA. In some embodiments, the mRNA is glycosylated. Additional mRNA modification and delivery approaches as described in Flynn et al., BioRxiv 787614 (2019) and Kowalski et al. Mol. Ther. 27(4): 710-728 (2019) are each incorporated herein by reference in its entirety.

### Myeloid Cells in Combination Therapy

Myeloid cell combination therapies are contemplated herein in which at least in one embodiment, myeloid cell therapy is employed prior to a CAR-T cell therapy, a checkpoint inhibitor therapy, a BiTE/TRiTE or other engager mediated therapy, NK cell therapy, monoclonal antibody therapy or multi-specific antibody therapy. In contrast, myeloid cell therapy can be employed, for example, concurrently with or following any of the therapies involving a CAR-T cell therapy, a checkpoint inhibitor therapy, a BiTE/TRiTE or other engager mediated therapy.

It is believed by the applicant of the instant invention disclosure, that myeloid cells can be employed to open up a window of access to tumor cells to various other therapies that are currently deemed less effective at least on an account of tumor accessibility or influence of the microenvironment. Myeloid cells engineered to attack the tumor can render the tumor at least transiently vulnerable to immune engagement. Engineered myeloid cells are designed for active chemotaxis and targeted phagocytosis and killing of tumor cells. In doing so, myeloid cell therapy facilitates access of other immune therapies to the tumor.

In some embodiments, a myeloid cell therapy is highly effective in killing at least 20% of tumor cells. In some embodiments, a myeloid cell therapy is highly effective in killing at least 30% of tumor cells. In some embodiments, a myeloid cell therapy is highly effective in killing at least 40% of tumor cells. In some embodiments, a myeloid cell therapy is highly effective in killing at least 50% of tumor cells. In some embodiments, a myeloid cell therapy is highly effective in killing at least 60% of tumor cells. In some embodiments, a myeloid cell therapy is highly effective in killing at least 70% of tumor cells.

In some embodiments, a myeloid cell therapy involves 1, 2, 3, 4, 5, 6 or 7 doses of the myeloid cells over a span of time. In some embodiments, the span of time is between 1 month and 6 months as is determined by a clinical practitioner and expert in the field. In some embodiments, at least 20%, at least 30%, at least 40%, at least 50%, at least 60% or at least 70% tumor cells are killed as a result of the myeloid cell therapy.

In some embodiments a second or subsequent therapy of the combination therapy that involves any one or more of a CAR-T cell therapy, a checkpoint inhibitor therapy, a BiTE/TRiTE or other engager mediated therapy, NK cell therapy, monoclonal antibody therapy or multi-specific antibody therapy is designed to follow once the myeloid cell therapy is completed, and may commence at least 8 days after the last dose of the myeloid cell therapy. In some embodiments, the second therapy commences at least 15 days or at least 30 days after the completion of the myeloid cell therapy. In some embodiments, the second therapy commences 1 month after the completion of the myeloid cell therapy. In some embodiments, the second therapy commences 2 months after the completion of the myeloid cell therapy. In some embodiments, the second therapy commences about 3-6 months after the completion of the myeloid cell therapy. In some embodiments, the second therapy commences within about 6 months after the completion of the myeloid cell therapy. In some embodiments, the second therapy commences within 12 months after the completion of the myeloid cell therapy.

In some embodiments the CAR-T cell therapy that follows the myeloid cell therapy may be directed to attack the same target antigen as the myeloid therapy, in other words, an ATAK myeloid cell for the myeloid cell therapy comprises a chimeric fusion protein that has an extracellular antigen binding domain that binds to an antigen on a target cell, e.g. cancer cell, and the chimeric antigen receptor (CAR) for the CAR-T cell therapy comprises an antigen binding domain that binds to the same antigen on the cancer cell, such that the CAR-T cell therapy augments the myeloid cell therapy in irradicating all cancer cells expressing the antigen. In some other embodiments, it may be envisioned that a myeloid cell therapy in which myeloid cells express a chimeric fusion protein with an extracellular domain that binds to a first cancer antigen on a cancer cell, is followed by a T cell therapy that comprises CARs that bind to a different antigen on the cancer cell. In some embodiments, a first therapy of the combination therapy described herein can be designed to target a first cell of the tumor or the tumor microenvironment, and the second therapy of the combination therapy may be directed to attack a different cell of the tumor or the microenvironment. In some embodiments, the first therapy or the second therapy is directed to metastizing cell or a pre-metastatic cell.

In some embodiments, the combination therapy comprising a myeloid cell therapy and a second therapy involving a CAR-T cell therapy, a checkpoint inhibitor therapy, a BiTE/TRiTE or other engager mediated therapy, NK cell therapy, monoclonal antibody therapy or multi-specific antibody therapy can be carried on in parallel at least for a duration of the therapy. In some embodiments, the myeloid cell therapy may be used as a preconditioning for a prolonged checkpoint inhibitor therapy or an antibody therapy.

In some embodiments, a monoclonal antibody, a cytokine or chemokine, a cytokine or chemokine inhibitor, a checkpoint inhibitor or a multispecific antibody can be used before commencement of, or continued concurrently with, or used after completion of a myeloid cell therapy.

In some embodiments, a checkpoint inhibitor therapy comprises an anti-PD-1 therapy. In some embodiments, a checkpoint inhibitor therapy comprises an anti-PD-L1 therapy. In some embodiments, a checkpoint inhibitor therapy comprises an anti-CTLA-4 therapy. In some embodiments, a checkpoint inhibitor therapy is directed against LAG3, 4-1BB, or OX40 therapy.

In some embodiments, an anti-CD47 therapy is used as a preconditioning for myeloid cell therapy.

In some embodiments, monoclonal antibody therapy comprises antibody therapy against any one or more of VEGF, EGFR, HER2, EpCAM, MUC-1 and others.

In some embodiments, the combination therapy acts through direct synergy or potentiation of the therapies, including increased antibody-dependent cellular phagocytosis (ADCP) and cytokine secretion.

### EXAMPLES

The present disclosure will be better understood with reference to the following examples. These examples are intended to representative of specific embodiments of the disclosure, and are not intended as limiting the scope of the disclosure.

### Example 1. A Phase 1/2, Open-Label, First-In-Human, Multiple Ascending Dose Multicenter Study of CD5-CFP Cell Pharmaceutical Composition in Subjects with CD5⁺ Relapsed/Refractory Peripheral T Cell Lymphoma

This example provides a study design for a Phase 1/2, Open-Label, Multiple Ascending Dose Multicenter Study of the CD5-CFP cell pharmaceutical composition in Subjects with CD5⁺ Relapsed/Refractory Peripheral T Cell Lymphoma.

CD5-CFP: The CD5-CFP cell is a CD5-directed genetically modified autologous myeloid cell immunotherapy for the treatment of CD5-expressing T cell lymphomas. The anti-CD5 CAR incorporated is composed of a humanized CD5-specific single-chain fragment variable (scFv) designed from the H65 murine monoclonal antibody, followed by a CD8 hinge and transmembrane region that is fused to the intracellular signaling domains for fragment crystallizable gamma (Fcγ) and phosphoinositide 3-kinase (PI3K), which is derived from CD19 intracellular signaling domains. In vitro engagement with the CD5 receptor results in signaling through the Fcγ and the PI3K domains. ATAK myeloid cells are advantageous over other CAR-engineered cells due to the following non-exhaustive list of attributes:
The ability to recognize and phagocytose tumor antigens resulting in tumor cell death and subsequent release of potential neoantigens.
The ability to present neoantigens to T cells resulting in the activation of novel tumor specific T cell clones.
The potential to activate, recruit, and synergize the multiple other aspects of the immune
response including αβ T cells, γδ T cells and natural killer (NK) cells as determined by the
expression of human leukocyte antigen and immune modulating chemokines and cytokines.
The ability to produce chemokine, cytokines and reactive oxygen species that are tumoricidal
and serve to recruit other elements of the anti-tumor response including NK cells, NK T cells
and γδ T cells.

The ATAK cells are produced by the transfection of mRNA into autologous myeloid cells resulting in transient expression of the CAR. This is different from CAR T cells which are produced by the transfection of lentivirus into autologous T cells resulting in long-term expression of the CAR. The design of the ATAK cells may limit the production of cytokines, thereby preventing CRS or limiting its duration.

About 21 up to 26 subjects will be recruited for the Phase 1 safety and efficacy evaluation of the pharmaceutical composition comprising engineered myeloid cells. A Phase 2 study will recruit about 17 evaluable participants.

Blood samples are collected from the patient, then myeloid cells are collected from this blood sample and are modified to express a binder to a protein on the surface of the malignant T cells and then reinfused back into the patient. The myeloid cells upon recognizing the malignant T cells can become activated and are designed to destroy the cancer cells.

The study is divided into 2 parts. The first part will be to determine the safety and tolerability of the study drug product. During this part of the study, there will be 3 groups of study patients. The first group of patients will receive a low dose of cells, the second group will receive a larger dose of cells, and the third group will receive the higher dose of cells and lymphodepleting chemotherapy to reduce the number of T cells in the blood. In the second part of the study, cells with or without chemotherapy will be administered depending on results of Part 1. The safety, tolerability and efficacy of MT-101 will be assessed. All patient groups will receive 6 doses of drug product over 3 weeks.

In order to balance safety versus potential for an effective dose in patients with median survival of less than 6 months, there is an intra-patient dose escalation in 1 patient in Cohort 1. This will be followed by enrollment of up to 10 subjects without the use of LD chemotherapy and up to 10 subjects with the use of LD chemotherapy. The initial dose in Cohort 1 is 10 times below the HED of the highest dose given in the murine safety study. The last dose is based on the number of cells which can be collected from a single leukapheresis and administered over 6 doses. The dose regimen of two doses in 3 sets across 16 days was based on the potential for an initial inflammatory reaction from the first dose with release of chemo-attractants specific for the myeloid cells, increasing trafficking of the cells to the tumor at the time of the second dose. The sets of doses are scheduled one week apart to account for the length of the transgene expression of up to 4 days and operational feasibility.

An average dose to be administered is 4.8×10\hat{ }8 cells per infusion.

Primary Objective(s): The primary objective of the Phase 1 portion of the study is to evaluate the safety and tolerability of the CD5-CFP cell pharmaceutical composition in subjects with CD5⁺ r/r PTCL at Day 28 to establish the maximum tolerated dose (MTD), and recommended Phase 2 dose (RP2D), based on observed AEs including all potential dose limiting toxicities (DLTs)

Secondary Objective(s): The secondary objectives of the Phase 1 portion of this study are to:
Determine CD5-CFP cell kinetics in the blood
Determine the objective response rate (ORR) (Complete Response (CR)+ Partial Response (PR)) according to the Lugano Classification criteria [Cheson 2014] at 6 months
Determine the duration of response (DOR)
Determine Progression Free Survival (PFS)
Determine Overall Survival (OS)
Determine rate of grade 3-5 Cytokine Release Syndrome (CRS)
Determine rate of immune effector cell-associated neurotoxicity syndrome (ICANS)

Exploratory Objective(s): The exploratory objectives of the Phase 1 portion of this study are to:
Assess the development of anti-drug antibodies (ADA) to the CD5-CFP cell pharmaceutical composition
Determine the heterogeneity of CD5 expression in tumors to identify potential biomarker of response
Determine the treatment related effects on cytokine and chemokine production, T cell receptor (TCR) expansion and cell phenotype in the blood to identify potential biomarkers of response.
Determine the treatment related effects to tumor architecture and tumor cell phenotype to identify potential biomarkers of response.

For Phase 2 studies, Primary Objective: The primary objective of the Phase 2 portion of the study is to determine the ORR (CR+PR) (Lugano Classification criteria 2014) in subjects with CD5⁺ r/r PTCL.

Secondary Objective(s): The secondary objectives of the Phase 2 portion of this study are to:
Determine the duration of response (DOR)
Determine the percent of subjects with a complete response (CR) at 6 months
Determine the percent of subjects with a partial response (PR) at 6 months
Determine the percent of subjects with stable disease (SD) at 6 months
Determine PFS
Determine OS
To further assess the safety and tolerability of the CD5-CFP cell pharmaceutical composition

Exploratory Objective(s) The exploratory objectives of the Phase 2 portion are the same as the Phase 1 portion of this study, plus: Assess quality of life (QOL) by the EuroQol-5D (EQ-5D) questionnaire

### Study Design

This is a multicenter, open-label, Phase 1/2, first-in-human (FIH) study to assess the safety, tolerability, and efficacy of the CD5-CFP cell pharmaceutical composition in subjects with CD5⁺ r/r PTCL. Subjects ≥18 years of age will be screened. Eligible subjects who provide written informed consent will be scheduled for a large volume leukapheresis prior to the first dose (Day 1).

### Phase 1

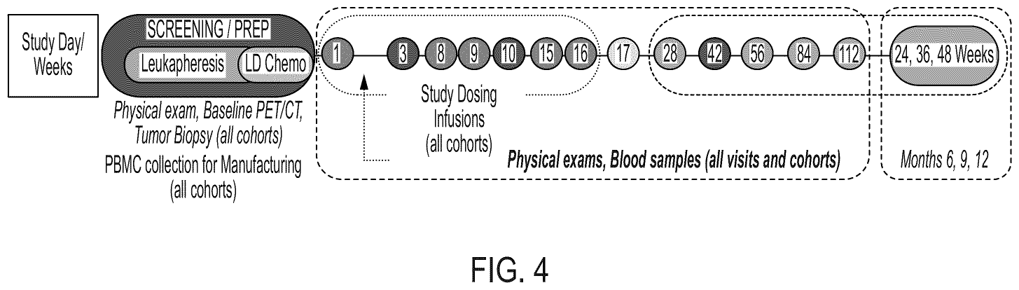

The Phase 1 portion of the trial will evaluate the safety and tolerability of the CD5-CFP cell pharmaceutical composition, with and without conditioning [lymphodepleting (LD) chemotherapy], for the purpose of identifying a dose and the use of LD chemotherapy for the Phase 2 portion of the trial. A subject intra-dose escalation will occur in one subject in the first cohort. The trial will be paused if a DLT occurs at any dose in the first cohort and an independent Data Monitoring Committee (DMC) will provide recommendations on continued dose escalation. The dose for Cohorts 2 and 3 will be determined after the DMC reviews the data from Cohort 1. The maximum dose will be 1.5×10⁸ cells. Patients will be followed for safety with DMC review after 3 subjects have completed Day 28 and 6 subjects have completed Day 28 in both Cohort 2 and Cohort 3. The DMC will make recommendations as to the continuation of dosing after each of these reviews. A representative scheme is shown in FIG. 4. Eligible subjects will be enrolled into the following dose cohorts:

| | Days −5, −4, −3 | Day 1 | Day 2 | Day 8 | Day 9 | Day 15 | Day 16 |
|---|---|---|---|---|---|---|---|
| Cohort | — | 0.5 × 10⁸ | — | 1.0 × 10⁸ | 1.0 × 10⁸ | 1.5 × 10⁸ | 1.5 × 10⁸ |
| 1 | | cells | | cells | cells | cells | cells |
| (n = 1) | | | | | | | |
| Cohort | — | 1.5 × 10⁸ | 1.5 × 10⁸ | 1.5 × 10⁸ | 1.5 × 10⁸ | 1.5 × 10⁸ | 1.5 × 10⁸ |
| 2 | | cells | cells | cells | cells | cells | cells |
| (n = 10) | | | | | | | |
| Cohort | LD | 1.5 × 10⁸ | 1.5 × 10⁸ | 1.5 × 10⁸ | 1.5 × 10⁸ | 1.5 × 10⁸ | 1.5 × 10⁸ |
| 3 | chemotherapy | cells | cells | cells | cells | cells | cells |
| (n = 10) | | | | | | | |

To monitor for acute and subacute adverse events (AEs), enrollment progression will occur as follows. Progression from dose cohort 1 will occur no sooner than completion of Day 28. Enrollment in dose cohorts 2 and 3 will occur in parallel, however, the first 3 subjects within these cohorts will be dosed a minimum of 14 days after the first dose of the prior subject (staggered).

The 10-point immune effector cell-associated encephalopathy (ICE) screening Table in Appendix 2 will be used for grading of potential ICANS. For a broader assessment CRS and ICANS associated AEs, use the ASTCT CRS grading Table in Appendix 2.

### Phase 2

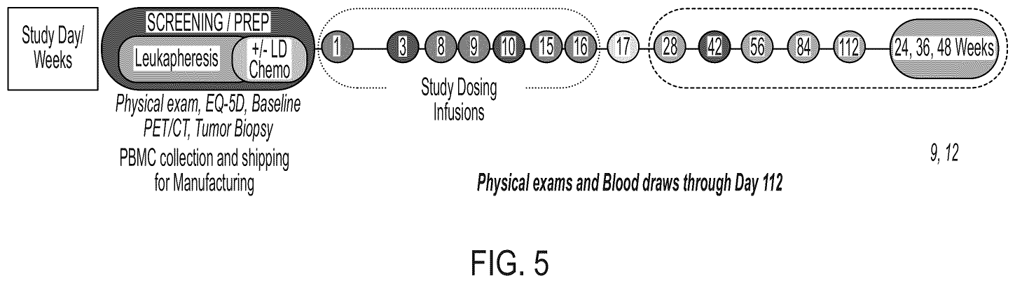

The dose for Phase 2 will be the maximum tolerated dose (MTD) based on DLT in Phase 1 of this study. Up to 17 subjects will receive the MTD from Phase 1 and the decision to administer LD chemotherapy will be dependent on the safety, efficacy and results of the correlative studies from Phase 1 portion of this study. A representative scheme is shown in FIG. 5.

### Follow-Up for all Subjects

In both portions of the study, subjects will be monitored for Adverse Events (AEs), infusion reactions (IRs), and vital signs for 8 hours after each infusion of the CD5-CFP cell pharmaceutical composition. Subjects will be assessed for safety and tolerability, including AEs, physical exam, vital signs, routine chemistry and hematology on Days 1, 2, 8, 9, 10, 15, 16, 17, 28, 42, and Months 2 and 4. Subjects will be monitored for clinical response by positron emission tomography—computed tomography (PET/CT) on Day 28, and Months 2, 4, 6, 9, and 12.

A tumor biopsy will be obtained for all subjects, except the subject in Cohort 1 (Phase 1), on Day 17. The biopsy will be used to determine if the CD5-CFP cell pharmaceutical composition cells have migrated to the tumor and to assess the change in the tumor microenvironment (including infiltration of T- and NK-cells). Whole blood will be obtained for ADA, cell phenotype, cell kinetics, TCR sequencing per the SOE Table 1 (Cohort 1) and Table 2 (Cohorts 2,3 and Phase 2). The EQ-5D will be administered in Phase 2 on Days 1, 15, 28, and Months 2, 4, 6, 9, and 12. All subjects will be followed for survival through Month 12.

### Conditioning Regimen [Lymphodepleting (LD) Chemotherapy]

Subjects assigned to Cohort 3 in Phase 1, and Phase 2 as described, will receive LD chemotherapy with fludarabine 25 mg/m² and cyclophosphamide 500 mg/m² on Days −5 through −3, prior to beginning dosing with the CD5-CFP cell pharmaceutical composition on Day 1. Subjects receiving conditioning will receive appropriate antibacterial and antiviral prophylaxis at the investigator's discretion.

### Dose Escalation or Addition of LD Chemotherapy During Phase 1

The DMC will review all available safety data after each dose escalation in Cohort 1, after the subject in Cohort 1 has completed Day 28 and after 3 and 6 subjects in Cohorts 2 and 3 have completed Day 28 The study stopping rules based on DLTs is outlined under the Statistical Considerations.

Continuation to Phase 2 with Recommended Dose and the Addition of LD Chemotherapy to the Treatment Regimen with CD5-CFP Cell Pharmaceutical Composition

The DMC will review all available safety, efficacy and biodistribution data after all subjects in Cohort 2 (10 evaluable subjects) and all subjects in Cohort 3 (10 evaluable subjects) have completed Day 28 and enrollment in the Cohort has not been stopped because of DLTs. The DMC will make one of the following recommendations:
Discontinue the study
Begin enrollment of Phase 2 with recommendations for dose and the use of LD chemotherapy based on the Maximum Tolerated Dose (MTD), efficacy and correlative studies. The MTD is one dose level below when DLTs occur in ≥33% of subjects.

Dose Limiting Toxicity Definition A DLT is defined using the Common Terminology Criteria for Adverse Events (CTCAE), v5. All toxicities will be considered “possibly” related to the CD5-CFP cell pharmaceutical composition unless they have no temporal association with the administration of the CD5-CFP cell pharmaceutical composition but rather related to other etiologies such as concomitant medications or conditions, or subject's underlying disease. For this study, the following are considered DLTs if they occur within 12 days of the last dose (Day 28):
Death
Any CTCAE Grade 4 toxicity
CTCAE Grade 3 toxicity in vital organs (heart, lung and central nervous system)
CTCAE Grade 3 toxicity that does not decrease to ≤grade 2 within 72 hours, except renal and hepatic abnormalities.
CTCAE Grade 3 toxicity that does not decrease to ≤Grade 2 within 7 days for renal and hepatic abnormalities.
Any ≥Grade 3 infusion related reaction lasting >24 hours
Any other toxicity which in the opinion of the DMC precludes further dosing

Route of Administration: The CD5-CFP cell pharmaceutical composition will be administered IV.

Duration of Treatment: There will be a total of 6 doses over 16 days; study follow up will proceed through 12 months.

### Subject Population

### Inclusion Criteria

Subjects are eligible for the study if all of the following criteria are met:
1. Females and males age ≥18 inclusive at the time the Informed Consent Form (ICF) is signed.
2. Refractory or relapsed pathologically confirmed TCL as defined by the following*:
   a. PTCL-NOS r/r to two lines of systemic therapy
   b. AITL r/r to two lines of systemic therapy
   c. ALK-negative ALCL r/r to two lines of systemic therapy which includes at least one line of therapy containing brentuximab, or
   d. ALK-positive ALCL r/r to two lines of systemic therapy which includes at least one line of therapy containing brentuximab.
   Subjects may have had autologous (not allogeneic) transplant as long as hematopoietic recovery has occurred.
3. CD5-expressing tumor by IHC or flow cytometry of tumor biopsy within 3 months of Screening or at Screening.
4. Measurable disease based on Lugano Classification criteria (Cheson, 2014)
5. Eastern Cooperative Oncology Group (ECOG) performance status grade of ≤2.
6. Life expectancy of >12 weeks.
7. Able to tolerate large volume leukapheresis, including adequate venous access.
8. Echocardiogram (ECHO) showing a left ventricular ejection fraction ≥40%.
9. Electrocardiogram (ECG) showing no clinically significant abnormality at Screening or showing an average QTc interval <450 msec in males and <470 msec in females (<480 msec for patients with bundle branch block). Either Fridericia's or Bazett's formula may be used to correct the QT interval.
10. Oxygen saturation of ≥94% on room air measured by pulse oximetry
11. Diffusing capacity of the lung for carbon monoxide (DLCO)≥50% of predicted
12. Adequate organ function as defined by the following laboratory values at Screening:
   Hemoglobin ≥8.0 g/dL without transfusion support for 7 days
   Platelet count ≥75,000/uL
   Absolute neutrophil count (ANC)>1000/mm 3 (without G-CSF support)
   Absolute lymphocyte count >400 mm 3
   Absolute monocytes >200 cells/mm 3
   Creatinine clearance >60 mL/min as calculated using the modification Cockcroft-Gault equation
   Alanine aminotransferase (ALT)<2×ULN; ALT <3 if known liver disease by PTCL
   Aspartate transaminase (AST)<2×ULN; AST <3 if known liver disease by PTCL
   Serum bilirubin <1.5×ULN except in patients with Gilbert's syndrome.
13. Willing and able to provide written informed consent.
14. Willing to perform and comply with all study procedures including attending clinic visits as scheduled.
15. Men and women of child bearing potential (WOCBP) must be willing to practice a highly effective method of contraception that may include, but is not limited to, abstinence, sex only with persons of the same sex, monogamous relationship with vasectomized partner, vasectomy, hysterectomy, bilateral tubal ligation, licensed hormonal methods, intrauterine device (IUD), or use of spermicide combined with a barrier method (e.g., condom, diaphragm) for 28 days before and after receiving the investigational product (IP).
16. Able to tolerate large volume leukapheresis, including adequate venous access.
17. Adequate organ function as indicated by the laboratory values in the clinical protocol.

### Exclusion Criteria

Subjects are excluded from the study if any of the following criteria are met:
1. Known central nervous system involvement by PTCL.
2. History of allogeneic transplant.
3. History of intolerance to leukapheresis, plasmapheresis, or blood donation.
4. Pregnant or nursing women.
5. Any acute illness including fever (>100.4° F. or >38° C.) within 14 days prior to Day 1.
6. Active systemic bacterial, fungal, or viral infection within 28 days prior to Day 1.
7. Active infection with human immunodeficiency virus (HIV), human T-lymphotropic virus (HTLV), hepatitis B virus (HBV), hepatitis C virus (HCV), or syphilis, as defined below:
   Positive serology for HIV-1 or 2, HTLV-1 or 2
   Positive serology for HCV and has not received a documented curative therapy
   Positive hepatitis B surface antigen (HBsAg) or Hepatitis B Core antibody (HBcAb).
   A positive nontreponemal and treponemal test for syphilis.
8. Other primary malignancies within 3 years of Screen, except:
   Adequately treated basal cell or squamous cell carcinoma
   In situ carcinoma of the cervix, breast or bladder, treated curatively and without evidence of recurrence for at least 3 years prior to the study, or
   A primary malignancy which has been completely resected and in complete remission for ≥5 years.
9. Active or history of autoimmune disease or immunodeficiency.
10. Evidence of current hemorrhagic cystitis.
11. History of severe, immediate hypersensitivity reaction attributed to penicillin.
12. History of symptomatic CHF (New York Heart Association [NYHA] classes II-IV) or serious active arrhythmias or other clinically significant cardiac disease within 12 months of enrollment.
13. Toxicity from previous anti-cancer therapy defined as toxicities (other than alopecia, or laboratory values listed above) not yet resolved to CTCAE Grade ≤1 or baseline. Subjects with chronic Grade 2 toxicities (e.g., peripheral neuropathy) may be eligible per the discretion of the Investigator and Medical Monitor.
14. Has received:
   Systemic corticosteroids within 7 days prior to leukapheresis
   Cytotoxic chemotherapy or other therapies for the treatment of TCL, within 14 days of leukapheresis
   Immune therapy (e.g., monoclonal antibody therapy, checkpoint inhibitors) within 5 half-lives or 3 weeks, whichever is shorter of leukapheresis, or
   Anti-cancer vaccine within 1 year
15. Has received a live vaccine ≤6 weeks prior to leukapheresis.
16. Enrollment in another interventional clinical trial within 30 days or 5 half-lives of the drug, whichever is shorter, prior to leukapheresis.
17. Has received autologous cell therapy.
18. Other active comorbidities that, in the opinion of the Investigator, would make the subject unsuitable for the study or unable to comply with the study requirements.
19. Any other condition that, in the opinion of the Investigator, would make the subject unsuitable for the study or unable to comply with the study requirements.

### Criteria for Evaluation

### (i) Safety Measures

AEs, IRs, serious AEs (SAES), events of special interest (AESI).
Vital signs
Serum chemistries and hematology

### (ii) Efficacy Measures

Clinical response assessed using Lugano Classification criteria (Cheson 2014) (PET/CT or Total Body CT)
   Biomarkers of response
   Blood
   Cytokine and chemokine production by Meso Scale Discovery (MSD)
   TCR sequencing
   Cell phenotype by mass cytometry (CyTOF)
   Tumor
   Tumor architecture by H&E
   Cell phenotype by mass cytometry (Hyperion), including:
      Monocytes (CD5-CFP cell pharmaceutical composition, CD14⁺), macrophages, dendritic cells
      CD4/CD8⁺ T cells (effector, memory, and Treg)
      Natural killer (NK) cells

Statistical Considerations: For Phase 1, Primary Endpoint: Safety

Number (%) of subjects experiencing at least one DLT will be displayed by cohort in Phase 1, using Dose Determine Population as denominator.

All subjects in the Safety Population will be included in the final summaries and listings of safety data Summaries of AEs and other safety parameters will be provided as appropriate. Emphasis in the analysis of AEs will be placed on those that are treatment-emergent through Day 28 days after first dose of CD5-CFP cell pharmaceutical composition on Day 1.

For Phase 2 Primary endpoint: ORR: ORR will be analyzed among the Efficacy Evaluable Population. ORR is defined as the number (%) of subjects achieving a best overall response of complete response (CR) or partial response (PR) by Lugano Classification criteria. The 95% exact confidence interval (CI) will also be calculated.

Simon's optimal 2-stage design is used in Phase 2. Interim analysis will be performed after the first stage. Cohort enrollment will be halted if needed while the stage 1 stopping rule is being evaluated. Eight efficacy evaluable subjects will be enrolled in the first stage. If 1 or fewer of those 8 subjects has a response, the study will be discontinued after stage 1. If 2 or more subjects have responses in a cohort, the cohort will continue to stage 2 with additional 9 subjects enrolled.

FIGS. 4 and 5 graphically represents some of the key elements described in this example.

### Example 2. CFP-Expressing ATAK Myeloid Cells Show Enhanced Phagocytosis when Combined with Anti-CD47 Treatment In Vitro

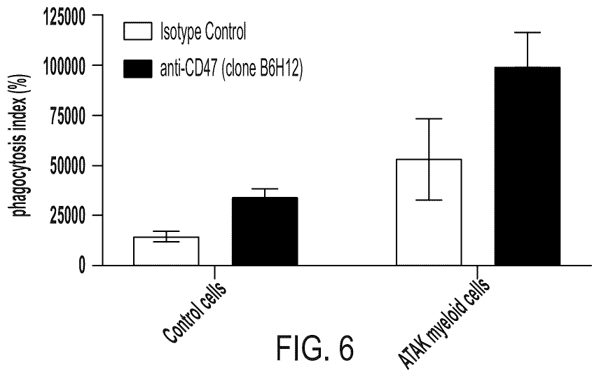

In this example, human CD14+ cells were isolated from leukopak leukapheresis sample and electroporated with a recombinant mRNA construct encoding CD5-FcG-PI3Kinase CFP (CD14+ ATAK cells). A CD5-FcG-PI3Kinase CFP comprises an extracellular antigen binding domain comprising an anti-CD5 scFv, CD8 hinge and transmembrane domains and intracellular domains comprising amino acid sequences from FcG intracellular signaling domain and from an intracellular PI3Kinase recruitment domain. CD14+ cells expressing CD5-FcG-PI3Kinase CFP are cocultured with target T cell lymphoma cell line (H9) for at least 3 hours and phagocytosis of the H9 cells by CFP expressing cells was assessed by flow cytometry. One set of cells from the above were treated with anti-CD47 antibody (Clone B6H12); and control cells were treated with the isotype control antibody. A comparison of the two sets of cells are shown in the representative data presented in FIG. 6. Combination of the CFP expressing cells with anti-CD47 antibody enhanced phagocytic killing of the target cells in vitro, compared to the isotype control.

### Example 3. Prophetic Example of Combination Therapy in a Mouse Xenograft of Human Endometrial Cancer Expressing HER2

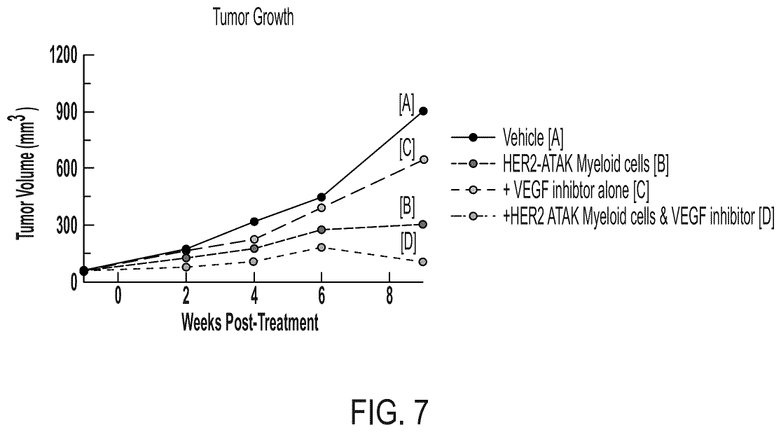

This is a prophetic example describing a study design and hypothesized outcome of combination therapy of VEGF inhibitor therapy and myeloid cell therapy (using ATAK myeloid cells) in treating cancer in a mouse xenograph model. Briefly, 6-8 week old, Crl: NU/NU-nuBR female athymic mice (NCI, Frederic, MD) are injected subcutaneously with 5×10⁶ cancer cells in 0.1 ml of RPMI cell culture medium using a syringe with a 22G5/8 needle to initiate a tumor. The control vehicle group has 8 mice and the treatment group has 16 mice. Therapeutic treatment is initiated when tumors are >75 mm\hat{ }3. ATAK myeloid cells are generated using bone marrow monocytes electroporated with a HER-2-FcG-PI3K-CD40 CAR. Bevacizumab reconstituted in saline is given at a dose of 0.2 mg/mouse through intraperitoneal (i.p.) injection twice a week, starting the day of cancer cell injection. 1-2×20\hat{ }6 ATAK cells are administered on days: 0, 1, 7, 8, 15 and 16 where day 0 in this scenario is the day the myeloid cells expressing HER2-specific chimeric fusion protein is first injected after the tumor attains the designated size. The control vehicle group is given saline intraperitoneally. Tumors are measured weekly with a caliper, and tumor cross-sectional areas (mm 2) are calculated using the formula: length (mm)×width (mm)×π/4. Data are analyzed by one-way ANOVA and Holm-Sidak T-test to compare the tumor sizes between control and treatment groups with a p-value of ≤0.05 considered to be significant. At sacrifice, tumors from control animals as well as those tumors that grew in the presence of bevacizumab are collected and snap-frozen in liquid nitrogen for RNA and protein extraction. FIG. 7 exemplifies a graphical representation of the likely outcome from the experiment described above.

### Example 4. Combination Therapy of Myeloid Cells with CAR-T Cells

This is a prophetic example describing a study design and hypothesized outcome of combination therapy of myeloid cell and CAR-T cell therapy in treating cancer in a mouse xenograph model. Briefly, 6-8 week old, Crl: C57BL/6 mice are injected subcutaneously with 5×10⁶ B16 cancer cells in 0.1 ml of RPMI cell culture medium using a syringe with a 22G5/8 needle. Murine CAR T cells can be generated by expressing CAR using retrovirus constructs. In this instance CAR-expressing constructs are generated by fusing geneblock fragments (custom ordered from IDT) into an MSCV retroviral vector. The complete second generation 28z CAR sequence is composed of a mouse CD8 signal peptide, antigen-specific scFv for GP75, mouse CD8a hinge and transmembrane domain, CD28 costimulatory domain and CD3 intracellular domain. The third generation 28BBz CAR can also be constructed similarly using a geneblock encoding the scFv followed by the CD8a transmembrane domain, CD28 costimulatory domain, 41BB costimulatory domain, and finally the CD3 intracellular domain.

### Virus Production

For optimal retrovirus production, 293 phoenix cells are cultured till 80% confluence, then split at 1:2 for further expansion. 24 hr later, 5.6×10⁶ cells are seeded in a 10 cm dish and cultured for 16 hr. 30 min-1 hr before transfection, each 10 cm dish is replenished with 10 ml pre-warmed medium. Transfection is carried out using the calcium phosphate method following the manufacturer's protocol (Clonetech). Briefly, for each transfection, 18 μg of plasmid (13.5 μg of CAR plasmid plus 4.5 μg of Eco packaging plasmid) is added to 610 μl of ddH₂O, followed by addition of 87 μl of 2 M CaCl₂). 700 μl of 2×HBS is then added in a dropwise manner with gentle vortexing. After a 5 min incubation at 25° C., the transfection mixture is gently added to phoenix cells. The next day, old medium is removed and replenished with 8 ml of pre-warmed medium without disturbing the cells. Virus-containing supernatant is collected 36 hr later and passed through a 0.45 um filter to remove cell debris. Virus supernatant is then aliquoted and stored at −80° C.

### Primary Mouse T Cell Isolation and CAR-T Cell Production

For T cell activation, 6-well plates are pre-coated with 5 ml of anti-CD3 (0.5 μg/ml, Clone: 2C11) and anti-CD28 (5 μg/ml, Clone: 37.51) per well at 4° C. for 18 hr. CD8+ T cells are isolated using a negative selection kit (Stem Cell Technology), and seeded onto pre-coated 6-well plates at 5×10⁶ cells/well in 5 ml of complete medium (RPMI+penicillin/streptomycin+10% FBS+1×NEAA+1× Sodium pyruvate+1× 2-mercaptoethanol+1×ITS). Cells are cultured at 37° C. for 48 hr without disturbance. 24 hr before transduction, non-TC treated plates are coated with 15 μg/ml of retronectin (Clonetech). On day 2, cells are collected, counted and resuspended at 2×10⁶ cells/ml in complete medium supplemented with 20 μg/ml of polybrene and 401 U/mL of mIL-2. Retronectin-coated plates are blocked with 0.05% FBS containing PBS for 30 min before use. 1 ml of virus supernatant is first added into each well of the blocked retronectin plate, then 1 mL of the above cell suspension is added and mixed well by gentle shaking to reach the working concentration of polybrene at 10 μg/ml and mIL-2 at 20 IU/ml. Spin infection is carried out at 2000×g for 120 min at 32° C. Plates are then carefully transferred to an incubator and maintained overnight. On day 3, plates are briefly centrifuged at 1,000×g for 1 min, and virus-containing supernatants are carefully removed. 3 mL of fresh complete medium containing 20 IU/ml of mIL-2 are then added into each well. Cells are passaged 1:2 every 12 hr with fresh complete medium containing 20 IU/mL of mIL-2. Transduction efficiency is evaluated by surface staining of c-Myc tag using an anti-Myc antibody (Cell signaling, Clone:9B11) 48 hr after transduction. For in vivo experiments, 0.1-10×20\hat{ }6 CAR-T cells are infused after 5 days of culture.

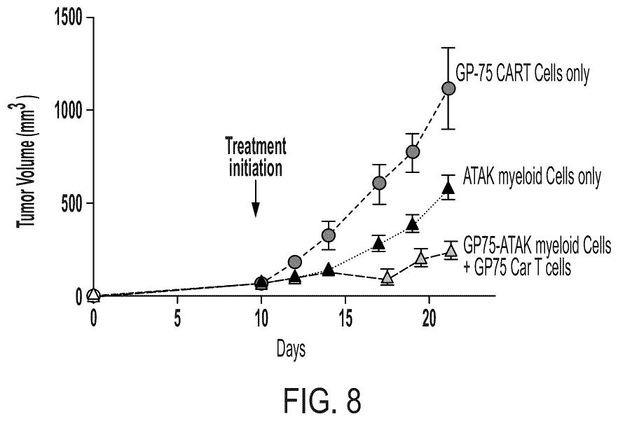

ATAK myeloid cells were generated using bone marrow monocytes electroporated with a GP75-FcG-PI3K-CD40 CAR, GP75-FcG-PI3K-TRIF CAR or GP75-FcG-PI3K. Approximately 1-2×20\hat{ }6 ATAK cells are administered on day 0, 1, 7, 8, 15 and 16 (FIG. 8).

### Example 5. In Vitro Assays for Testing Myeloid Cell Competency

The following are some exemplary assay methods that can be used to test myeloid cell efficacy.

### Phagocytosis Assay:

Antigen-linked silica or polystyrene beads ranging in diameters 1 nm, 5 nm or 10 nm were used for a screen of macrophages. Inert beads are coated in a supported lipid bilayer and the antigens are ligated to the lipid bilayer. J774 macrophage cell lines are prepared, each cell line expressing a cloned recombinant plasma membrane protein. The recombinant plasma membrane protein may also express a fluorescent tag. The cell lines are maintained and propagated in complete RPMI media with heat inactivated serum and antibiotics (Penicillin/Streptomycin). On the day of the assay, cells are plated at a density of 1×10\hat{ }6 cells/ml per well in 6 well plates or in a relative proportion in 12 or 24 well plates, and incubated for 2-6 hours. The cells are then washed once in Phosphate Buffer Saline, and the beads are added in serum depleted or complement depleted nutrient media. Cells are visualized by light microscopy at 30 minutes and 2 hours after addition of the beads Immunofluorescence reaction may be performed using tagged antibody, and fluorescent confocal microscopy is used to detect the interaction and co-localization of cellular proteins at engulfment. Confidence levels are determined by Kruskal-Wallis test with Dunn's multiple comparison correction.

In some examples, dye loaded tumor cells are fed to macrophage cell lines and phagocytosis is assessed by microscopy.

### Cytokine Production:

Macrophage cell lines are cultured as above. In one assay, each J774 cell line expressing a plasma membrane protein is plated in multi-wells and challenged with antigen-linked beads and cytokine production was assayed by collecting the supernatants at 4 hours and 24 hours. Cytokines are assayed from the supernatant by ELISA. In another fraction, cells are collected at 4 and 24 hours after incubation with the beads and flow cytometry is performed for detection of cytokines. In each case, multiple cytokines are assayed in a multiplex format, which can be selected from: IL-1α, IL-1β, IL-6, IL-12, IL-23, TNF-α, GMCSF, CXCL1, CXCL3, CXCL9, CXCL-10, MIP1-α and MIP-2. Macrophage inflammatory cytokine array kit (R&D Systems) is used.

Intracellular signaling pathway for inflammatory gene and cytokine activation can be identified by western blot analysis for phosphorylation of MAP kinases, JNK, Akt signaling pathway, Interferon activation pathway including phosphorylation and activation of STAT-1.

### Inflammasome Activation Assay

Activation of NLRP3 inflammasome is assayed by ELISA detection of increased IL-1 production and detection caspase-1 activation by western blot, detecting cleavage of procaspase to generate the shorter caspase. In a microwell plate multiplex setting, Caspase-Glo (Promega Corporation) is used for faster readout of Caspase 1 activation.

iNOS Activation Assay:

Activation of the oxidative burst potential is measured by iNOS activation and NO production using a fluorimetric assay NOS activity assay kit (AbCAM).

### Cancer Cell Killing Assay:

Raji B cells are used as cancer antigen presenting cells. Raji cells are incubated with whole cell crude extract of cancer cells, and co-incubated with J774 macrophage cell lines. The macrophages can destroy the cells after 1 hour of infection, which can be detected by microscopy or detected by cell death assay.

1.-143. (canceled)

144. A method of treating a cancer in a human subject in need thereof that has been treated with an immune cell inhibitory agent, the method comprising administering to the human subject a pharmaceutical composition comprising:
(I) a population of cells comprising a therapeutically effective amount of CD14+ cells comprising a recombinant polynucleic acid, wherein the recombinant polynucleic acid comprises a sequence encoding a chimeric fusion protein (CFP), the CFP comprising:
(i) an extracellular domain comprising an antigen binding domain,
(ii) a transmembrane domain operatively linked to the extracellular domain, and
(iii) an intracellular domain comprising one or more intracellular signaling domains; and
(II) a pharmaceutically acceptable carrier.

145. The method of claim 144, wherein the method further comprises administering the immune cell inhibitory agent to the human subject prior to administering the pharmaceutical composition.

146. The method of claim 144, wherein the immune cell inhibitory agent comprises a lymphodepletion agent.

147. The method of claim 144, wherein the immune cell inhibitory agent comprises trabectedin.

148. The method of claim 144, wherein the immune cell inhibitory comprises lurbinectedin.

149. The method of claim 146, wherein the lymphodepletion agent comprises fludarabine.

150. The method of claim 147, wherein the lymphodepletion agent comprises cyclophosphamide.

151. The method of claim 146, wherein the lymphodepletion agent comprises fludarabine and cyclophosphamide.

152. The method of claim 151, wherein the fludarabine is administered for 2, 3, 4, 5, 6, or 7 days prior to administering the pharmaceutical composition and the cyclophosphamide is administered for 1, 2, 3, 4, or 5 days prior to administering the pharmaceutical composition.

153. The method of claim 151, wherein the fludarabine is administered at a dose of from 20-30 mg/m².

154. The method of claim 151, wherein the cyclophosphamide is administered at a dose of from 200-2000 mg/m².

155. The method of claim 151, wherein the subject is administered about 25 mg/m² fludarabine and about 500 mg/m² cyclophosphamide on Days −5 through −3 prior to administering the pharmaceutical composition.

156. The method of claim 144, wherein the CFP comprises an antigen binding domain that binds to a cancer antigen CD5 on a cancer cell.

157. The method of claim 144, wherein the antigen binding domain comprises an anti-CD5 single chain variable domain (scFv), wherein the anti-CD5 scFv comprises:
(i) a heavy chain variable domain (VH), the heavy chain variable domain comprising a heavy chain complementarity determining region 1 (HC CDR1) sequence of NYGMN (amino acid residues 31-35 of SEQ ID NO: 37), a HC CDR2 sequence of WINTHTGEPTYADSFKG (amino acid residues 50-66 of SEQ ID NO: 37) and a HC CDR3 sequence of RGYDWYFDV (amino acid residues 99-107 of SEQ ID NO: 37); and
(ii) a light chain variable domain (VL), the light chain variable domain comprising a light chain complementarity determining region 1 (LC CDR1) sequence of RASQDINSYLS (amino acid residues 24-34 of SEQ ID NO: 2), a LC CDR2 sequence of RANRLES (amino acid residues 50-56 of SEQ ID NO: 2) and a LC CDR3 sequence of QQYDESPWT (amino acid residues 89-97 of SEQ ID NO: 2).

158. The method of claim 157, wherein the anti-CD5 scFv comprises a VH sequence with at least 85% sequence identity to SEQ ID NO: 37, and a VL sequence with at least 85% sequence identity to SEQ ID NO: 2.

159. The method of claim 158, wherein the anti-CD5 scFv comprises a sequence with at least 85% sequence identity to SEQ ID NO: 33.

160. The method of claim 144, wherein the extracellular domain is from CD8 and comprises a sequence with at least 85% sequence identity to SEQ ID NO: 7.

161. The method of claim 144, wherein the transmembrane domain is from CD8 and comprises a sequence with at least 85% sequence identity to SEQ ID NO: 6.

162. The method of claim 144, wherein the intracellular domain comprises an intracellular signaling domain from FcRγ and comprises a sequence with at least 85% sequence identity to SEQ ID NO: 27.

163. The method of claim 162, wherein the intracellular domain further comprises a PI3K recruitment domain that has at least 85% sequence identity to SEQ ID NO: 4.

164. The method of claim 144, wherein the CFP comprises a sequence with at least 85% sequence identity to SEQ ID NO: 14.

165. The method of claim 144, wherein the cancer is T cell lymphoma.

166. The method of claim 165, wherein the T cell lymphoma peripheral T cell lymphoma (PTCL), relapsed peripheral T cell lymphoma, or a refractory peripheral T cell lymphoma.

167. The method of claim 144, wherein the CD14+ cells are autologous CD14+ cells.

168. A pharmaceutical composition comprising:
(a) an immune cell inhibitory agent, and
(b) a population of cells comprising a therapeutically effective amount of CD14+ cells comprising a recombinant polynucleic acid, wherein the recombinant polynucleic acid comprises a sequence encoding a chimeric fusion protein (CFP), the CFP comprising:
(i) an extracellular domain comprising an antigen binding domain,
(ii) a transmembrane domain operatively linked to the extracellular domain, and
(iii) an intracellular domain comprising one or more intracellular signaling domains.


SEQUENCE LISTING (54 sequences)

SEQ ID NO: 1 - protein, 116 aa, synthetic construct
  REGION 1..116: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 2 - protein, 107 aa, synthetic construct
  REGION 1..107: note = Description of Artificial Sequence: Synthetic polypeptide
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 3 - protein, 46 aa, synthetic construct
  REGION 1..46: note = Description of Artificial Sequence: Synthetic polypeptide
LYCRRLKIQVRKAAITSYEKSDGVYTGLSTRNQETYETLKHEKPPQ

SEQ ID NO: 4 - protein, 35 aa, synthetic construct
  REGION 1..35: note = Description of Artificial Sequence: Synthetic polypeptide
YEDMRGILYAAPQLRSIRGQPGPNHEEDADSYENM

SEQ ID NO: 5 - protein, 62 aa, synthetic construct
  REGION 1..62: note = Description of Artificial Sequence: Synthetic polypeptide
KKVAKKPTNKAPHPKQEPQEINFPDDLPGSNTAAPVQETLHGCQPVTQEDGKESRISVQERQ

SEQ ID NO: 6 - protein, 21 aa, synthetic construct
  REGION 1..21: note = Description of Artificial Sequence: Synthetic peptide
IYIWAPLAGTCGVLLLSLVIT

SEQ ID NO: 7 - protein, 62 aa, synthetic construct
  REGION 1..62: note = Description of Artificial Sequence: Synthetic polypeptide
ALSNSIMYFSHFVPVFLPAKPTTTPAPRPPTPAPTIASQPLSLRPEACRPAAGGAVHTRGLD

SEQ ID NO: 8 - protein, 130 aa, synthetic construct
  REGION 1..130: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 9 - protein, 108 aa, synthetic construct
  REGION 1..108: note = Description of Artificial Sequence: Synthetic polypeptide
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 10-13 - skipped (4 entries)

SEQ ID NO: 14 - protein, 432 aa, synthetic construct
  REGION 1..432: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 15 - protein, 436 aa, synthetic construct
  REGION 1..436: note = Description of Artificial Sequence: Synthetic polypeptide
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 16 - protein, 454 aa, synthetic construct
  REGION 1..454: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQKKVAKKPTNKAPHPKQEPQEINFPDDLPGSNTAAPVQETLHGCQPVTQEDGKESRISVQERQ

SEQ ID NO: 17 - protein, 17 aa, synthetic construct
  REGION 1..17: note = Description of Artificial Sequence: Synthetic peptide
MWLQSLLLLGTVACSIS

SEQ ID NO: 18 - protein, 27 aa, synthetic construct
  REGION 1..27: note = Description of Artificial Sequence: Synthetic peptide
FWVLVVVGGVLACYSLLVTVAFIIFWV

SEQ ID NO: 19 - protein, 25 aa, synthetic construct
  REGION 1..25: note = Description of Artificial Sequence: Synthetic peptide
ILLPLIIGLILLGLLALVLIAFCII

SEQ ID NO: 20 - protein, 45 aa, synthetic construct
  REGION 1..45: note = Description of Artificial Sequence: Synthetic polypeptide
LYCRLKIQVRKAAITSYEKSDGVYTGLSTRNQETYETLKHEKPPQ

SEQ ID NO: 21 - protein, 219 aa, synthetic construct
  REGION 1..219: note = Description of Artificial Sequence: Synthetic polypeptide
QRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 22 - protein, 171 aa, synthetic construct
  REGION 1..171: note = Description of Artificial Sequence: Synthetic polypeptide
PLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 23 - protein, 211 aa, synthetic construct
  REGION 1..211: note = Description of Artificial Sequence: Synthetic polypeptide
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 24 - protein, 607 aa, synthetic construct
  REGION 1..607: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSMSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 25 - protein, 615 aa, synthetic construct
  REGION 1..615: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 26 - protein, 567 aa, synthetic construct
  REGION 1..567: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 27 - protein, 42 aa, synthetic construct
  REGION 1..42: note = Description of Artificial Sequence: Synthetic polypeptide
RLKIQVRKAAITSYEKSDGVYTGLSTRNQETYETLKHEKPPQ

SEQ ID NO: 28 - skipped

SEQ ID NO: 29 - protein, 24 aa, synthetic construct
  REGION 1..24: note = Description of Artificial Sequence: Synthetic peptide
IYIWAPLAGTCGVLLLSLVITLYC

SEQ ID NO: 30 - protein, 86 aa, synthetic construct
  REGION 1..86: note = Description of Artificial Sequence: Synthetic polypeptide
ALSNSIMYFSHFVPVFLPAKPTTTPAPRPPTPAPTIASQPLSLRPEACRPAAGGAVHTRGLDIYIWAPLAGTCGVLLLSLVITLYC

SEQ ID NO: 31 - protein, 83 aa, synthetic construct
  REGION 1..83: note = Description of Artificial Sequence: Synthetic polypeptide
ALSNSIMYFSHFVPVFLPAKPTTTPAPRPPTPAPTIASQPLSLRPEACRPAAGGAVHTRGLDIYIWAPLAGTCGVLLLSLVIT

SEQ ID NO: 32 - protein, 255 aa, synthetic construct
  REGION 1..255: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESSGGGGSGGGGSGGGGSLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 33 - protein, 240 aa, synthetic construct
  REGION 1..240: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 34 - protein, 26 aa, synthetic construct
  REGION 1..26: note = Description of Artificial Sequence: Synthetic peptide
IYLIIGICGGGSLLMVFVALLVFYIT

SEQ ID NO: 35 - DNA, 10 bp, synthetic construct
  misc_feature 1..10: note = Description of Artificial Sequence: Synthetic oligonucleotide
  source 1..10: mol_type = other DNA
gccrccatgg

SEQ ID NO: 36 - protein, 17 aa, synthetic construct
  REGION 1..17: note = Description of Artificial Sequence: Synthetic peptide
LQVTDSGLYRCVIYHPP

SEQ ID NO: 37 - protein, 118 aa, synthetic construct
  REGION 1..118: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 38 - protein, 257 aa, synthetic construct
  REGION 1..257: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 39 - protein, 107 aa, synthetic construct [sequence omitted]

SEQ ID NO: 40 - protein, 120 aa, synthetic construct
  REGION 1..120: note = Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 41 - protein, 244 aa, synthetic construct
  REGION 1..244: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 42 - protein, 191 aa, synthetic construct
  REGION 1..191: note = Description of Artificial Sequence: Synthetic polypeptide
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 43 - protein, 247 aa, synthetic construct
  REGION 1..247: note = Description of Artificial Sequence: Synthetic polypeptide
QVQLVQSGAEVKKPGASVKVSCKASGYTFTNYGMNWVRQAPGQGLEWMGWINTYTGEPTYADAFKGRVTMTTDTSTSTAYMELRSLRSDDTAVYYCARDYGDYGMDYWGQGTTVTVSSGSTSGSGKPGSSEGSTKGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 44 - protein, 247 aa, synthetic construct
  REGION 1..247: note = Description of Artificial Sequence: Synthetic polypeptide
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAYSLGERATINCKSSQSLFNSGNQKNYLTWYQQKPGQPPKLLIYWASTRESGVPDRFSGSGSGTDIFITISSLQAEDVAVYYCQNAYSFPYTFGGGTKLEIKR

SEQ ID NO: 45 - protein, 244 aa, synthetic construct
  REGION 1..244: note = Description of Artificial Sequence: Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 46 - protein, 244 aa, synthetic construct
  REGION 1..244: note = Description of Artificial Sequence: Synthetic polypeptide
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 47 - protein, 121 aa, synthetic construct
  REGION 1..121: note = Description of Artificial Sequence: Synthetic polypeptide
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 48 - protein, 108 aa, synthetic construct
  REGION 1..108: note = Description of Artificial Sequence: Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 49 - protein, 115 aa, synthetic construct
  REGION 1..115: note = Description of Artificial Sequence: Synthetic polypeptide
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSS

SEQ ID NO: 50 - protein, 112 aa, synthetic construct
  REGION 1..112: note = Description of Artificial Sequence: Synthetic polypeptide
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 51 - protein, 242 aa, synthetic construct
  REGION 1..242: note = Description of Artificial Sequence: Synthetic polypeptide
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 52 - protein, 25 aa, synthetic construct
  REGION 1..25: note = Description of Artificial Sequence: Synthetic peptide
ILLPLIIGLILLGLLALVLIAFAII

SEQ ID NO: 53 - protein, 43 aa, synthetic construct
  REGION 1..43: note = Description of Artificial Sequence: Synthetic polypeptide
RRLKIQVRKAAITSYEKSDGVYTGLSTRNQETYETLKHEKPPQ

SEQ ID NO: 54 - protein, 35 aa, synthetic construct
  REGION 1..35: note = Description of Artificial Sequence: Synthetic polypeptide
MHEEEIYTSLQWDSPAPDTYQKCLSSNKCSGACCL